(19) United States
(12) Patent Application Publication
(10) Pub. No.: US 2021/0236513 A1
(43) Pub. Date: Aug. 5, 2021

(54) NOVEL USES

(71) Applicant: ZARODEX THERAPEUTICS LIMITED, London (GB)

(72) Inventors: Stephen JOLLES, Cardiff (GB); Houman ASHRAFIAN, London (GB); Duncan MCHALE, London (GB)

(73) Assignee: ZARODEX THERAPEUTICS LIMITED, London (GB)

(21) Appl. No.: 16/965,248

(22) PCT Filed: Jan. 31, 2019
(86) PCT No.: PCT/EP2019/052449

§ 371 Date: Jul. 27, 2020

(30) Foreign Application Priority Data
Jan. 31, 2018 (EP) 18154553.4

(51) Int. Cl. A61K 31/5513; A61K 45/06; A61P 19/02

(52) U.S. Cl. CPC A61K 31/5513; A61P 19/02; A61K 45/06

(57) ABSTRACT

This invention relates to the compound clozapine and its major metabolite norclozapine and prodrugs thereof and pharmaceutically acceptable salts and solvates thereof for use in the treatment or prevention of a pathogenic immunoglobulin driven B cell disease. The invention also provides pharmaceutical compositions containing such compounds.

### TECHNICAL FIELD

This invention relates to a compound and pharmaceutical compositions containing such compound for use in the treatment or prevention of a pathogenic immunoglobulin driven B cell disease.

### BACKGROUND TO THE INVENTION

The compound associated with this invention is known as clozapine i.e. the compound of the following structure:

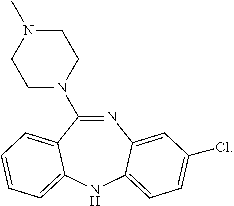 [SMILES: CN1CCN(C2=Nc3cc(Cl)ccc3Nc3ccccc32)CC1]

Clozapine has a major active metabolite known as norclozapine (Guitton et al., 1999) which has the following structure:

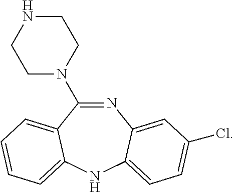 [SMILES: Clc1ccc2c(c1)N=C(N1CCNCC1)c1ccccc1N2]

Clozapine is known as a treatment for resistant schizophrenia. Schizophrenia is an enduring major psychiatric disorder affecting around 1% of the population. Apart from the debilitating psychiatric symptoms it has serious psychosocial consequences with an unemployment rate of 80-90% and a life expectancy reduced by 10-20 years. The rate of suicide among people with schizophrenia is much higher than in the general population and approximately 5% of those diagnosed with schizophrenia commit suicide.

Clozapine is an important therapeutic agent and is included on the WHO list of essential medicines. It is a dibenzo-diazepine atypical antipsychotic, and since 1990 the only licensed therapy in the UK for the 30% of patients with treatment-resistant schizophrenia (TRS). It shows superior efficacy in reducing both positive and negative symptoms in schizophrenic patients and is effective in approximately 60% of previously treatment refractive patients with a significant reduction in suicide risk. The National Institute for Health and Clinical Excellence (NICE) guideline recommends adults with schizophrenia which has not responded adequately to treatment with at least 2 antipsychotic drugs (at least one of which should be a non-clozapine second generation antipsychotic) should be offered clozapine.

Clozapine is associated with serious adverse effects including seizures, intestinal obstruction, diabetes, thromboembolism, cardiomyopathy and sudden cardiac death. It can also cause agranulocytosis (cumulative incidence 0.8%); necessitating intensive centralised registry based monitoring systems to support its safe use. In the UK there are three electronic registries (www.clozaril.co.uk, www.denzapine.co.uk and www.ztas.co.uk) one for each of the clozapine suppliers. Mandatory blood testing is required weekly for the first 18 weeks, then every two weeks from weeks 19-52 and thereafter monthly with a ‘red flag’ cut-off value for absolute neutrophil count (ANC) of less than 1500/μL for treatment interruption.

In 2015, the Federal Drug Administration (FDA) merged and replaced the six existing clozapine registries in the United States combining data from over 50,000 prescribers, 28,000 pharmacies and 90,000 patients records into a single shared registry for all clozapine products, the Clozapine Risk Evaluation and Mitigation Strategy (REMS) Program (www.clozapinerems.com). Changes were introduced lowering the absolute neutrophil count (ANC) threshold to interrupt clozapine treatment at less than 1000/μL in general, and at less than 500/μL in benign ethnic neutropenia (BEN). Prescribers have greater flexibility to make patient-specific decisions about continuing or resuming treatment in patients who develop moderate to severe neutropenia, and so maximize patient benefit from access to clozapine.

Schizophrenia is associated with a 3.5 fold increased chance of early death compared to the general population. This is often due to physical illness, in particular chronic obstructive pulmonary disease (COPD) (Standardised Mortality Ratio (SMR) 9.9), influenza and pneumonia (SMR 7.0). Although clozapine reduces overall mortality in severe schizophrenia, there is a growing body of evidence linking clozapine with elevated rates of pneumonia-related admission and mortality. In an analysis of 33,024 patients with schizophrenia, the association between second generation antipsychotic medications and risk of pneumonia requiring hospitalization was highest for clozapine with an adjusted risk ratio of 3.18 with a further significant increase in risk associated with dual antipsychotic use (Kuo et al., 2013). Although quetiapine, olanzapine, zotepine, and risperidone were associated with a modestly increased risk, there was no clear dose-dependent relationship and the risk was not significant at time points beyond 30 days (Leung et al., 2017; Stoecker et al., 2017).

In a 12 year study of patients taking clozapine, 104 patients had 248 hospital admissions during the study period. The predominant admission types were for treatment of either pulmonary (32.2%) or gastrointestinal (19.8%) illnesses. The commonest pulmonary diagnosis was pneumonia, (58% of pulmonary admissions) and these admissions were unrelated to boxed warnings (Leung et al., 2017).

In a further nested case control study clozapine was found to be the only antipsychotic with a clear dose-dependent risk for recurrent pneumonia, this risk increased on re-exposure to clozapine (Hung et al., 2016).

While these studies underscore the increased admissions or deaths from pneumonia and sepsis in patients taking clozapine over other antipsychotics, the focus on extreme outcomes (death and pneumonia) may underestimate the burden of less severe but more frequent infections such as sinusitis, skin, eye, ear or throat infections and community acquired and treated pneumonia. Infection may represent an important additional factor in destabilizing schizophrenia control and clozapine levels.

Various mechanisms for the increase in pneumonia have been suggested, including aspiration, sialorrhoea and impairment of swallowing function with oesophageal dilatation, hypomotility and agranulocytosis. In addition, cigarette smoking is highly prevalent among patients with schizophrenia as a whole and represents an independent risk factor for pneumonia incidence and severity (Bello et al., 2014).

A small amount of research into the immunomodulatory properties of clozapine has been performed:

Hinze-Selch et al (Hinze-Selch et al., 1998) describes clozapine as an atypical antipsychotic agent with immunomodulatory properties. This paper reports that patients that received clozapine treatment for six weeks showed significant increases in the serum concentrations of IgG, but no significant effect was found on IgA or IgM concentrations or on the pattern of autoantibodies.

Jolles et al (Jolles et al., 2014) reports studies on the parameter “calculated globulin (CG)” as a screening test for antibody deficiency. Patients with a wide range of backgrounds were selected from thirteen laboratories across Wales. Of the patients with significant antibody deficiency (IgG <4 g/L, reference range 6-16 g/L), identified on CG screening from primary care, clozapine use was mentioned on the request form in 13% of the samples. However, antibody deficiency is not a listed side effect of clozapine in the British National Formulary (BNF), nor does antibody testing constitute part of current clozapine monitoring protocols.

Another study by Lozano et al. (Lozano et al., 2016) reported an overall decrease of mean plasma levels of IgM in the study group (which consisted of psychiatric outpatients who took clozapine for at least five years) compared to the control group, and also reported that no differences were found between the groups with respect to IgA, IgG, absolute neutrophil count and white blood cell count.

Consequently, given these mixed results that have been reported, the immunomodulatory properties of clozapine and its effect on immunoglobin levels are neither clear nor understood in the art.

Pathogenic immunoglobulin (including IgG, IgA and IgM) driven diseases result from secretion of autoantibodies (principally IgG and/or IgA) by antibody secreting cells (“ASCs”, collectively plasmablasts and plasma cells, these being types of mature B cell). These antibodies target a variety of self-antigens which have been characterised in many of these conditions. There is rarely an increase in overall immunoglobulins as the pathological process is driven by the secretion of specific immunoglobulins which constitute a small percentage of the total immunoglobulins. Secretion of IgG antibodies and IgA antibodies are from ASCs, and ASCs are generated secondary to the differentiation of class-switched and unswitched memory B cells, these being further types of mature B cell. Various lines of evidence suggest this is a highly-dynamic process, with ongoing differentiation occurring almost constantly.

Class-switched memory B cells are mature B cells that have replaced their primary encoded membrane receptor [IgM] by IgG, IgA or IgE in response to repeated antigen recognition. This class-switching process is a key feature of normal humoral immunological memory, both ‘constitutive’ through the secretion of pre-existing protective antibodies by long-lived plasma cells, and ‘reactive’ reflecting re-exposure to antigen and reactivation of memory B cells to either differentiate into plasma cells to produce antibodies, or to germinal centre B cells to enable further diversification and affinity maturation of the antibody response. Early in the immune response, plasma cells derive from unswitched activated B cells and secrete IgM. Later in the immune response, plasma cells originate from activated B cells participating in the germinal centre (areas forming in secondary lymphoid follicular tissue in response to antigenic challenge) which have undergone class switching (retaining antigen specificity but exchanging immunoglobulin isotype) and B cell receptor (BCR) diversification through immunoglobulin somatic hypermutation. This maturation process enables the generation of BCRs with high affinity to antigen and production of different immunoglobulin isotypes (i.e. exchanging the originally expressed IgM and IgD to IgG, IgA or IgE isotypes) (Budeus et al., 2015; Kracker and Durandy, 2011).

Class switch recombination (CSR) following the germinal centre reaction in secondary lymphoid organs provides antigen-primed/experienced autoreactive memory B cells and a core pathway for development and/or maintenance of autoimmunity. Post-germinal centre B cells class-switched to IgG or IgA in the periphery can enter other anatomic compartments, such as the central nervous system, to undergo further affinity maturation (e.g. in tertiary lymphoid structures in multiple sclerosis) and contribute to immune pathology (Palanichamy et al., 2014). CSR can occur locally within tissue in pathology, such as within ectopic lymphoid structures in chronically inflamed tissue such as rheumatoid arthritis synovium (Alsaleh et al., 2011; Humby et al., 2009).

A significant proportion of bone marrow plasma cells are IgA⁺ (^{˜}40%) with IgA⁺ plasma cells further constituting the majority in serum (^{˜}80%) (Mei et al., 2009) consistent with a substantial contribution of IgA⁺ plasma cells to the bone marrow population of long-lived cells. The intestinal mucosa is the primary inductive site for IgA⁺ plasma cells, mainly through gut-associated lymphoid tissue (GALT, comprising Peyer's patches and isolated lymphoid follicles) (Craig and Cebra, 1971), together with mesenteric lymph nodes and, potentially, the intestinal lamina propria itself, with class-switch recombination towards IgA achieved through both T cell-independent (pre-germinal centre formation) (Bergqvist et al., 2010; Casola et al., 2004) and T cell-dependent mechanisms (Pabst, 2012). Notably, IgA⁺ and other plasma cells (in addition to plasmablasts) are increasingly understood to exert important effector immune functions beyond the production of immunoglobulin, including generation of cytokines (Shen and Fillatreau, 2015) and immunoregulators such as tumour-necrosis factor-α (TNF-α), inducible nitric oxide synthase (iNOS) (Fritz et al., 2011), IL-10 (Matsumoto et al., 2014; Rojas et al., 2019), IL-35 (Shen et al., 2014), IL-17a (Bermejo et al., 2013) and ISG15 (Care et al., 2016).

Plasmablasts, representing short-lived rapidly cycling antibody-secreting cells of the B cell lineage with migratory capacity, are also precursors to long-lived (post-mitotic) plasma cells, including those which home in to the bone marrow niche (Nutt et al., 2015). In addition to being precursors of autoreactive long-lived plasma cells, plasmablasts are an important potential therapeutic target themselves through their ability to produce pathogenic immunoglobulin/autoantibody (Hoyer et al., 2004), particularly IgG but also IgM, described in several disease contexts such as neuromyelitis optica (Chihara et al., 2013; Chihara et al., 2011), idiopathic pulmonary arterial hypertension, IgG4-related disease (Wallace et al., 2015), multiple sclerosis (Rivas et al., 2017) and transverse myelitis (Ligocki et al., 2013), rheumatoid arthritis (Owczarczyk et al., 2011) and systemic lupus erythematosus (SLE) (Banchereau et al., 2016). In addition to their direct antibody secreting function, circulating plasmablasts also exert activity to potentiate germinal centre-derived immune responses and thereby antibody production via a feed-forward mechanism involving II-6-induced promotion of T follicular helper cell (Tfh) differentiation and expansion (Chavele et al., 2015).

Long-lived plasma cells, whose primary residency niche is in bone marrow (Benner et al., 1981), are thought to be the major source of stable autoantibody production in (both physiologic) and pathogenic states and are resistant to glucocorticoids, conventional immunosuppressive and B cell depleting therapies (Hiepe et al., 2011). Substantiating the critical importance of this B cell population to long-term antibody production, site-specific survival of bone marrow-derived plasma cells with durable (up to 10 years post-immunisation) antibody responses to prior antigens has been demonstrated in non-human primates despite sustained memory B cell depletion (Hammarlund et al., 2017). Given the key role played by autoreactive long-lived plasma cells in the maintenance of autoimmunity (Mumtaz et al., 2012)—and the substantial refractoriness of the autoreactive memory formed by these cells to conventional immunosuppressive agents such as anti-TNF or B cell depleting biologics (Hiepe et al., 2011)

CD19(+) B cells and CD19(−) B plasma cells are drivers of pathogenic immunoglobulin driven B cell diseases. In particular, pathogenic IgG and IgA driven B cell diseases represent a substantial proportion of all autoimmune diseases. The most prominent, but not the sole mechanism through which pathogenic immunoglobulin driven B cells cause disease, is through auto-antibody production. Established pathogenic IgG immunoglobulin diseases include Pemphigus and Pemphigoid. Pemphigus, which has been designated to be an orphan disease, is an autoimmune interepithelial blistering disease characterised by loss of normal cell-cell adhesion (acantholysis). The antibodies involved are against desmoglein 3. If left untreated, it can be fatal, usually from overwhelming opportunistic infection due to loss of skin barrier function and from electrolyte loss. Pemphigoid is characterised by the formation of blister at the space between the epidermis and dermis skin layers. The antibodies involved are against dystonin and/or type XVII collagen.

Pathogenic immunoglobulin driven B cell diseases are poorly treated and as a result they have substantial mortality and morbidity rates, even for the “benign” diseases. Certain current advanced therapies are directed at mature B cells. For example, belimumab is a human monoclonal antibody that inhibits B cell activating factor. Atacicept is a recombinant fusion protein that also inhibits B cell activating factor. However, memory B cells may be resistant to therapies such as belimumab or atacicept which target survival signals such as B cell activation factor (Stohl et al., 2012). The importance of memory B cells in the pathogenesis of autoimmune disorders was also demonstrated by the lack of efficacy of atacicept in treating rheumatoid arthritis and multiple sclerosis (Kappos et al., 2014; Richez et al., 2014). Plasmapheresis and immunoabsorption involve the removal of disease-causing autoantibodies from the patient's bloodstream. However, these treatments have limited efficacy or are complex and costly to deliver. CAR-T methods directed at CD19(+) B cells leaves CD19(−) B plasma cells intact, which makes it ineffective.

Rituximab is a drug that is currently used to treat some pathogenic IgG driven B cell diseases. It targets B cells that express CD20. However, CD20 is only expressed on a limited subset of B cells. It also does not target plasma cells. This limited expression of CD20 and lack of effect on plasma cells explains the limited efficacy of rituximab in a variety of diseases, both benign and malignant, despite being definitively of B cell origin. Rituximab does not appear to have any effect on IgA-secreting plasmablasts/plasma cells, and consequently the associated IgA driven B cell diseases (Yong et al., 2015).

Thus, there is a major unmet medical need for new treatments against pathogenic immunoglobulin driven B cell diseases.

### SUMMARY OF THE INVENTION

### Impact on Class-Switched Memory B Cells and Antibody Production

It has been found by the inventors that clozapine has a potential important therapeutic effect as it significantly reduces class switched memory B cells (“CSMB”), a type of mature B cell.

Reduction in CSMBs by clozapine will consequently reduce the numbers of ASCs, and hence the secretion of specific immunoglobulins including the pathogenic immunoglobulins. Clozapine was also observed to cause a reduction in levels of plasmablasts, another type of mature B cell. This functional effect on persistent and long lived adaptive B cell and plasma cell function may ameliorate the diseases driven by the persistent generation of pathogenic immunoglobulins that drives the pathology of pathogenic immunoglobulin driven B cell diseases. The inventors' new data demonstrates a very significant effect on the number of circulating class switched memory B cells, a substantial effect on the number of plasmablasts and importantly, through the lack of recall response to common vaccines, an effect on the function of the class switched memory B cells and plasmablasts resulting in specific reduction of antibodies targeting a previously exposed antigen. The inventors' new data also demonstrates an effect of the drug in reducing total IgG, IgA and IgM levels after administration. With the lack of effect on other B cells, shown by the lack of depletion of other sub-types and total B cell numbers but with a particular reduction in CSMBs and plasmablasts, this observation strongly supports a functional effect on CSMBs and plasmablasts which are central to long lived production of pathogenic antibodies in pathogenic immunoglobulin (particularly IgG and IgA) driven B cell diseases.

The inventors' finding of a marked reduction in class-switched memory B cells in patients treated with clozapine indicates a robust impact on the process of immunoglobulin class switching. This has particular therapeutic relevance in pathogenic immunoglobulin driven B cell diseases in which class switch recombination (CSR) following the germinal centre reaction in secondary lymphoid organs provides antigen-primed/experienced autoreactive memory B cells and a core pathway for development and/or maintenance of autoimmunity. Further, this also has particular therapeutic relevance since B lymphoid kinase haplotypes associated with B cell-driven autoimmune disorders exhibit an expansion of class-switched memory B cells and disease models of intrinsic B cell hyperactivity are associated with spontaneous CSR as associated with high titres of IgG autoantibodies The effect of clozapine to both impact on CSR and lower IgG is of especial therapeutic potential in the setting of pathogenic immunoglobulin-driven B cell diseases where an impact on both the autoimmune memory repertoire and pathogenic immunoglobulin is desirable.

Impact on IgA

The inventors have identified a significantly reduced circulating total IgA in patients treated with clozapine (leftward shift in immunoglobulin distribution) which notably demonstrated disproportionate lowering of IgA compared to that found with IgG and IgM. Substantiating the functional impact of this, the inventors have also identified a highly significant reduction in pneumococcal-specific IgA in patients treated with clozapine compared to clozapine-naive patients taking other antipsychotics. Recapitulating this in a model mammalian system, the inventors demonstrate that dosing of wild type mice with clozapine results in a significant reduction in circulating IgA compared to control or haloperidol treatment. While present at a relatively lower concentration in plasma compared to other immunoglobulin isotypes, IgA forms the great majority of all mammalian immunoglobulin, with ^{˜}3 g/day produced in human.

The inventors' finding of a significant reduction in total IgA in response to clozapine treatment reflects an important effect of clozapine on the function of IgA⁺ plasma cells. The generation of such cells occurs in both bone marrow and intestinal mucosae.

The inventors' identification of a significant impact of clozapine on plasma cell populations indicates the clear potential to modulate the diverse antibody-independent effector functions of B cells relevant to (auto)immune-mediated disease also.

### Impact on Plasmablast Antibody-Secreting Cells

The inventors have found that clozapine exerts a profound effect on reducing levels of circulating plasmablasts in patients. Accordingly, the inventors' observation of a profound impact of clozapine use on circulating plasmablast number highlights the potential for clozapine to modulate pathogenic immunoglobulin-driven B cell disease through both effects on circulating plasmablast secretion of immunoglobulin as well as interference with the potent function of plasmablasts to promote Tfh function.

### Impact on Long-Lived Plasma Cells

Using a wild type murine model, the inventors have found that regular clozapine administration in mice significantly reduces the proportion of long-lived plasma cells in bone marrow, an effect not seen with use of a comparator antipsychotic agent (haloperidol). Notably, human bone marrow resident long-lived PCs are long-regarded as the primary source of circulating IgG in human, thus providing a clear substrate for the inventors' observation of reduction in IgG in patients treated with clozapine. The inventors' observation of a specific effect of clozapine to deplete bone marrow long-lived plasma cells suggests it has, via an impact on long-lived plasma cell (autoreactive) memory, substantial therapeutic potential in pathogenic immunoglobulin driven B cell disease to eliminate inflammation and achieve remission.

### Impact on B Cell Precursors in Bone Marrow and Splenic Immature/Transitional Cells

The inventors identify a clear impact of clozapine on bone marrow B cell precursors after dosing of wild type mice. Specifically, an increase in the proportion of pre-pro B cells, in conjunction with a reduction in pre-B cells, proliferating pre-B cells and immature B cells in bone marrow. Together, these findings suggest a specific impact of clozapine on early B cell development, with a partial arrest between the pre-pro-B cell and pre-B cell stages in the absence of specific immunological challenge. The inventors have discerned an impact of clozapine to reduce the proportion of splenic T1 cells in wild type mice. Mirroring the murine findings, the inventors' interim findings from an ongoing observational study of patients on clozapine reveal a significant reduction in circulating transitional B cells. The human circulating transitional B cell subpopulation exhibits a phenotype most similar to murine T1 B cells and is expanded in patients with SLE.

Accordingly, the inventors' observation of an impact of clozapine to reduce the proportions of bone marrow B cell progenitors and immature (T1) splenic B cells provides additional anatomic compartmental origins beyond germinal centres for their finding of a reduction in circulating class-switched memory B cells and immunoglobulin in patients treated with clozapine. The therapeutic potential of this is further underlined by the consideration that the majority of antibodies expressed by early immature B cells are self-reactive.

### Lack of Direct B Cell Toxicity In Vitro

The inventors' new data using an in vitro B cell differentiation system to assess the specific impact of clozapine, its metabolite (N-desmethylclozapine) and a comparator antipsychotic control drug (haloperidol) further demonstrate: no direct toxicity effect of clozapine or its metabolite on differentiating B cells, no consistent effect on the ability of differentiated ASCs to secrete antibody and no consistent inhibitory effect on functional or phenotypic maturation of activated B cells to an early PC state in the context of an established in vitro assay.

Limited to the context of these in vitro experiments, these data suggest that clozapine is unlikely to be acting in a direct toxic manner on plasma cells or their precursors (e.g. via a cell intrinsic effect) to induce the effects observed on immunoglobulin levels. The observations suggest that clozapine's effect on B cells is more nuanced than existing B cell targeting therapies used for autoimmune disease which result in substantial depletion of multiple B cell subpopulations (e.g. rituximab and other anti-CD20 biosimilars) whose efficacy is mediated via direct effects on B cells such as signalling induced apoptosis, complement-mediated cytotoxicity or antibody-dependent cellular cytotoxicity.

Such a lack of apparent substantial direct toxicity by clozapine has a number of potential therapeutic advantages for clozapine, including reduced risk of generalised immunosuppression associated with indiscriminate B cell depletion (including elimination of protective B cells), and the potential to avoid maladaptive alterations observed with use of conventional B cell depleting therapies.

### Efficacy in Collagen-Induced Arthritis (CIA) Mouse Model and Relevance of CIA as a Model of B Cell Driven Disease Involving Pathogenic Immunoglobulin

CIA is a well-established experimental model of autoimmune disease that results from immunisation of genetically susceptible strains of rodents and non-human primates with type II collagen (CII) (Brand et al., 2004)—a major protein component of cartilage—emulsified in complete Freund's adjuvant. This results in an autoimmune response accompanied by a severe polyarticular arthritis, typically 18-28 days post-immunisation and monophasic, resolving after ^{˜}60 days in mice (Bessis et al., 2017; Brand et al., 2007). The pathology of the CIA model resembles that of rheumatoid arthritis, including synovitis, synovial hyperplasia/pannus formation, cartilage degradation, bony erosions and joint ankylosis (Williams, 2012).

The immunopathogenesis of CIA is dependent on B cell-specific responses with generation of pathogenic autoantibodies to CII, in addition to involving T cell-specific responses to CII, FcγR (i.e. Fc receptors for IgG) and complement. The critical role of B cells in the development of CIA is substantiated by the complete prevention of development of CIA in mice deficient for B cells (IgM deleted), notwithstanding an intact anti-CII T cell response (Svensson et al., 1998). Moreover, the development of CIA has been shown to be absolutely dependent on germinal centre formation by B cells, with anti-CII immunoglobulin responses themselves largely dependent on normal germinal centre formation (Dandah et al., 2018; Endo et al., 2015). B cells have also been implicated in other aspects of CIA pathology, including bone erosion through inhibition of osteoblasts (Sun et al., 2018). As a corollary, B cell depletion using anti-CD20 monoclonal antibodies prior to CII immunisation delays onset and severity of CIA, in conjunction with delayed autoantibody production (Yanaba et al., 2007). In this model, B cell recovery was sufficient to result in pathogenic immunoglobulin production after collagen-immunisation and associated development of disease.

The fundamental role played by collagen-specific IgG autoantibodies in the pathogenesis of CIA are highlighted by the observations that passive transfer of anti-CII serum or polyclonal IgG immunoglobulin to unimmunised animals results in arthritis (Stuart and Dixon, 1983), whilst lack of the FcγR chain near completely abrogates development of CIA in mice (Kleinau et al., 2000). In addition, introduction of pathogenic antibodies (i.e. collagen antibody-induced arthritis, CAIA) into germinal centre-deficient mice results in arthritis, demonstrating the ability of pathogenic antibody to largely circumvent the requirement for the germinal centre reaction (Dandah et al., 2018). Moreover, even mice lacking adaptive immunity (i.e. B and T cells), are susceptible to induction of CIA (Nandakumar et al., 2004).

Accordingly, the inventors have employed the CIA model as a highly clinically relevant experimental system in which B cell-derived pathogenic immunoglobulin made in response to a sample specific antigen drives autoimmune pathology to explore the potential efficacy of clozapine and its associated cellular mechanisms. The inventors demonstrate that clozapine delays the onset and reduces the incidence of CIA in mice, an effect most apparent when dosed just after CII immunisation. Furthermore, the inventors' data indicates that clozapine reduces the severity of CIA, judged by number of affected paws and clinical severity score. The inventors identify important effects of clozapine on key cell types implicated in the pathogenesis of CIA, including a reduction in the proportion of splenic plasma cells and highly significant reduction in germinal centre B cells in local draining lymph node. Moreover, the inventors' findings demonstrate reduced markers of functional activity for antibody production and antigen presentation on lymph node germinal centre B cells in response to clozapine in CII immunised mice. Measured at a single time point, they also observe a significant reduction in anti-collagen IgG1 antibody levels. Together, the inventors' findings in the CIA model point to a specific ability of clozapine to favourably impact upon pathogenic immunoglobulin B cell-driven pathology and thereby B cell mediated disorders in which autoantibody formation is a key component.

Thus, the present invention provides a compound selected from clozapine, norclozapine and prodrugs thereof and pharmaceutically acceptable salts and solvates thereof for use in the treatment or prevention of a pathogenic immunoglobulin driven B cell disease in a subject, in particular, wherein said compound causes mature B cells to be inhibited in said subject.

### BRIEF DESCRIPTION OF THE DRAWINGS

FIGS. 1A-C. show the relative frequencies of numbers of patients at each serum concentration value for IgG, IgA and IgM respectively for clozapine-treated patients (black) and clozapine-naïve patients (grey) (see Example 1).

FIG. 1D illustrates density plots showing the distribution of serum immunoglobulin levels in patients receiving clozapine referred for Immunology assessment (light grey left-most curve, n=13) following removal of 4 patients (n=2 with haematological malignancy and n=2 previously included within the inventor's recent case-control study (Ponsford et al., 2018)). Serum immunoglobulin distributions for clozapine-treated (mid-grey middle curve, n=94) and clozapine-naive (dark grey right-most curve, n=98) are also shown for comparison [adapted from (Ponsford et al., 2018)]. Dotted lines represent the 5th and 95th percentiles for healthy adults (see Example 1).

FIG. 2. shows the effect of duration of clozapine use on serum IgG levels (see Example 1).

FIG. 3A. shows the number of class switched memory B cells (CSMB) (CD27+/IgM−/IgD−, expressed as a percentage of total CD19+ cells) in healthy controls, in patients taking clozapine referred to clinic and in patients with common variable immunodeficiency disorder (CVID) (see Example 1).

FIG. 3B. shows B cell subsets, expressed as a percentage of total CD19⁺ cells, in patients with schizophrenia with a history of clozapine therapy referred to clinic (numbers as shown), common variable immunodeficiency (CVID, n=26) and healthy controls (n=17). B-cell subsets gated on CD19⁺ cells and defined as follows: Naïve B-cells (CD27⁻IgD⁺IgM⁺), Marginal Zone-like B-cells (CD27⁺IgD⁺IgM⁺), Class-switched Memory B-cells (CD27⁺IgD⁻IgM⁻), and Plasmablasts (CD19⁺CD27^{Hi}IgD⁻). Non-parametric Mann-Whitney testing performed for non-normally distributed data, * p<0.05, ** p<0.01, *** p<0.001, ****p<0.0001 (see Example 1).

FIG. 4A. shows the number of plasmablasts (CD38+++/IgM−, expressed as a percentage of total CD19+ cells) in healthy controls, in patients taking clozapine referred to clinic and in patients with common variable immunodeficiency disorder (CVID) (see Example 1).

FIG. 4B. illustrates vaccine specific-IgG response assessment (see Example 1).

FIG. 5. shows gradual recovery of serum IgG post-discontinuation of clozapine from 3.5 to 5.95 g/L over three years. LLN=lower limit of normal (see Example 1).

FIG. 6A-C. shows interim data findings on the levels of circulating IgG, IgA and IgM in patients on non-clozapine antipsychotics (‘control’, left) versus clozapine (right). Mean±SEM (see Example 2).

FIG. 7. shows interim data findings on peripheral blood levels of pneumococcal-specific IgG in patients on non-clozapine antipsychotics (‘control’, left) versus clozapine (right). Mean±SEM (see Example 2).

FIG. 8A-B. shows interim data findings on peripheral blood levels of B cells (CD19⁺) in patients on non-clozapine antipsychotics (‘control’, left) versus clozapine (right), expressed as absolute levels and as a percentage of lymphocytes (%, i.e. of T+B+NK cells). Mean±SEM (see Example 2).

FIG. 9A-C. shows interim data findings on peripheral blood levels of naive B cells (CD19⁺/CD27⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 10A-C. shows interim data findings on peripheral blood levels of memory B cells (CD19⁺/CD27⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 11A-C. shows interim data findings on peripheral blood levels of class switched (CS) memory B cells (CD27⁺/IgM⁻/IgD⁻) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 12A-C. shows interim data findings on peripheral blood levels of IgM high IgD low (CD27⁺/IgM⁺⁺/IgD⁻) memory B cells, i.e. post-germinal centre IgM only B cells, in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 13A-C. shows interim data findings on peripheral blood levels of transitional B cells (IgM⁺⁺/CD38⁺⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 14A-C. shows interim data findings on peripheral blood levels of marginal zone (MZ) B cells (CD27⁺/IgD⁺/IgM⁺) in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 15A-C. shows interim data findings on peripheral blood levels of plasmablasts in patients on non-clozapine antipsychotics (‘control’, left) versus clozapine (right), expressed as a percentage of total B cells (CD19⁺ cells, % B), lymphocytes (% L), or absolute values (abs), respectively. Mean±SEM (see Example 2).

FIG. 16. shows the body weight growth curve of WT mice in response to clozapine at different doses versus haloperidol and vehicle controls. Mean±SEM (see Example 3).

FIG. 17. shows body weight comparisons of WT mice at days 3, 12 and 21 of treatment. Mean±SEM (see Example 3).

FIG. 18. shows the impact of clozapine versus haloperidol and vehicle control on overall B cell content and pre-pro B cell and pro B cell precursors in bone marrow of WT mice. Mean±SEM (see Example 3).

FIG. 19. shows the impact of clozapine versus haloperidol and vehicle control on pre-B cells, proliferating B cells and immature B cell precursors in bone marrow of WT mice. Mean±SEM (see Example 3).

FIG. 20. shows the impact of clozapine versus haloperidol and vehicle control on class-switched memory B cells, plasmablasts and long-lived plasma cells in bone marrow of WT mice. Mean±SEM (see Example 3).

FIG. 21. shows the impact of clozapine versus haloperidol and vehicle control on overall B cells, T cells, other cell populations (TCR-β⁻/B220⁻) and activated T cells in spleen of WT mice. Mean±SEM (see Example 3).

FIG. 22. shows the impact of clozapine versus haloperidol and vehicle control on transitional (T1 and T2), follicular, marginal zone (MZ) and germinal centre (GC) B cells in spleen of WT mice. Mean±SEM (see Example 3).

FIG. 23. shows the impact of clozapine versus haloperidol and vehicle control on B cell subpopulations and T cells in the mesenteric lymph nodes (MLN) of WT mice. Mean±SEM. T1 and T2, transitional type 1 and type 2 B cells, respectively. MZ, marginal zone. GC, germinal centre (see Example 3).

FIG. 24. shows the impact of clozapine versus haloperidol and vehicle control on circulating immunoglobulins in WT mice. Mean±SEM (see Example 3).

FIG. 25. shows impact of clozapine on day of clinical onset of CIA. Mean±SEM (see Example 4).

FIG. 26. shows impact of clozapine on incidence of CIA (see Example 4).

FIG. 27. shows the impact of clozapine on the severity of CIA, judged by clinical score and thickness of first affected paw, in mice dosed from day 1 post-immunisation. Mean±SEM (see Example 4).

FIG. 28. shows the impact of clozapine on the severity of CIA, judged by number of affected paws by day of treatment with clozapine (day 15, D15 or day 1, D1) post-immunisation. Mean±SEM (see Example 4).

FIG. 29. shows the impact of clozapine versus control on B220⁺ (i.e. CD45⁺) cells in spleen and local lymph node of CIA mice. Mean±SEM (see Example 4).

FIG. 30. shows the impact of clozapine versus control on plasma cells (PC) in spleen and local lymph node of CIA mice. Mean±SEM (see Example 4).

FIG. 31. shows the impact of clozapine versus control on germinal centre (GC) B cells (B220⁻/IgD⁻/Fas⁺/GL7⁺) in spleen and local lymph node of CIA mice. Mean±SEM (see Example 4).

FIG. 32. shows the impact of clozapine versus control on expression of GL7 on germinal centre (GC) B cells (B220⁺/IgD⁻/Fas⁺/GL7⁺) in spleen and local lymph node of CIA mice. MFI, mean fluorescent intensity. Mean±SEM (see Example 4).

FIG. 33. shows the impact of clozapine versus control on peripheral blood anti-collagen IgG1 and IgG2a antibody levels of CIA mice (see Example 4).

FIG. 34. shows the impact of clozapine versus control on germinal centre resident T follicular helper cells (CD4⁺ PD1⁺) in spleen and local lymph node of CIA mice. Mean±SEM (see Example 4).

FIG. 35. shows the impact of clozapine versus control on expression of PD1 on germinal centre resident T follicular helper cells (CD4⁺ PD1⁺) in spleen and local lymph node of CIA mice. MFI, mean fluorescent intensity. Mean±SEM (see Example 4).

FIG. 36. shows the impact of clozapine versus control on expression of CXCR5 on germinal centre resident T follicular helper cells (CD4⁺ PD1⁺) in spleen and local lymph node of CIA mice. MFI, mean fluorescent intensity. Mean±SEM (see Example 4).

FIG. 37. shows the impact of clozapine versus control on expression of CCR7 on germinal centre resident T follicular helper cells (CD4⁺ PD1⁺) in spleen and local lymph node of CIA mice. MFI, mean fluorescent intensity. Mean±SEM (see Example 4).

FIG. 38. shows protocol schematic for in vitro generation/differentiation of human plasma cells (see Example 5).

FIG. 39. shows a schematic of the trial illustrating clozapine uptitration period followed by administration of typhoid vaccine (Typhim Vi) by injection (arrow) and then ongoing dosing with clozapine. Control cohort (vaccine only, no clozapine) and optional cohort (dose to be selected guided by findings from dose 1 and dose 3) (see Example 6).

### DETAILED DESCRIPTION OF THE INVENTION

The present invention also provides a method of treatment or prevention of a pathogenic immunoglobulin driven B cell disease in a subject by administering to said subject an effective amount of a compound selected from clozapine, norclozapine and prodrugs thereof and pharmaceutically acceptable salts and solvates thereof, in particular, wherein said compound causes mature B cells to be inhibited in said subject.

The present invention also provides use of a compound selected from clozapine, norclozapine and prodrugs thereof and pharmaceutically acceptable salts and solvates thereof in the manufacture of a medicament for the treatment or prevention of a pathogenic immunoglobulin driven B cell disease in a subject, in particular, wherein said compound causes mature B cells to be inhibited in said subject.

Clozapine or norclozapine may optionally be utilised in the form of a pharmaceutically acceptable salt and/or solvate and/or prodrug. In one embodiment of the invention clozapine or norclozapine is utilised in the form of a pharmaceutically acceptable salt. In a further embodiment of the invention clozapine or norclozapine is utilised in the form of a pharmaceutically acceptable solvate. In a further embodiment of the invention clozapine or norclozapine is not in the form of a salt or solvate. In a further embodiment of the invention clozapine or norclozapine is utilised in the form of a prodrug. In a further embodiment of the invention clozapine or norclozapine is not utilised in the form of a prodrug.

The term “pathogenic immunoglobulin driven B cell disease” includes B cell mediated disease, especially autoimmune disease, which involves pathogenic immunoglobulins (e.g. IgG, IgA and/or IgM) targeting a self-antigen (e.g. auto-antibody IgG, IgA and/or IgM) as a principal mechanism.

Suitably the pathogenic immunoglobulin driven B cell disease is a pathogenic IgG driven B cell disease. Alternatively, suitably it is a pathogenic IgA driven B cell disease.

The term “pathogenic IgG driven B cell disease” includes B cell mediated disease, especially autoimmune disease, which involves pathogenic IgG targeting a self-antigen (i.e. auto-antibody IgG) as a principal mechanism.

The term “pathogenic IgA driven B cell disease” includes B cell mediated disease, especially autoimmune disease, which involves pathogenic IgA targeting a self-antigen (i.e. auto-antibody IgA) as a principal mechanism.

The range of self-antigens involved in autoimmune diseases include desmoglein 3, BP180, BP230, (pemphigus), dystonin and/or type XVII collagen (pemphigoid), myelin (multiple sclerosis), pancreatic beta cell proteins (Type 1 diabetes mellitus), nicotinic acetylcholine receptors (myasthenia gravis), neuronal surface proteins (autoimmune epilepsy and encephalitis), 2-hydrolase (autoimmune Addison's disease), FcεRI (chronic autoimmune urticaria) and acetylcholine receptor (myasthenia gravis).

The range of self-antigens involved in pathogenic IgA driven B cell diseases include tissue transglutaminase (dermatitis herpetiformis and coeliac disease), gliadin IgA (coeliac disease) and dystonin and/or type XVII collagen (linear IgA disease).

Exemplary pathogenic IgG driven B cell diseases are autoimmune diseases including those which may be selected from the group consisting of the skin related diseases Pemphigus vulgaris, Pemphigus foliaceus, bullous pemphigoid, cicatricial pemphigoid, autoimmune alopecia, vitiligo, dermatitis herpetiformis and chronic autoimmune urticaria. Alternatively, the disease may be the gut related disease coeliac disease. Alternatively, the diseases may be selected from the group consisting of the thyroid gland related diseases Graves' disease and Hashimoto's thyroiditis. Alternatively, the diseases may be the pancreas related disease Type 1 diabetes mellitus. Alternatively, the disease may be the adrenal gland related disease autoimmune Addison's disease. Alternatively, the diseases may be selected from the group consisting of the haematological related diseases autoimmune haemolytic anaemia, autoimmune thrombocytopenic purpura and cryoglobulinemia. Alternatively, the disease may be the gut related disease pernicious anaemia. Alternatively, the diseases may be selected from the group consisting of the neurological related diseases myasthenia gravis, multiple sclerosis, neuromyelitis optica and autoimmune epilepsy and encephalitis. Alternatively, the diseases may be selected from the group consisting of the liver related diseases autoimmune hepatitis, primary biliary cirrhosis and primary sclerosing cholangitis.

References highlighting the role of B cells and pathogenic antibodies in the aforementioned diseases are provided below:

Pemphigus vulgaris and Pemphigus foliaceus

Pemphigus is a B cell-mediated autoimmune blistering disease of the skin and mucosa characterised by the generation of pathogenic autoantibodies, predominantly of the IgG4 subclass (but also IgG1 and less so IgA) (Futei et al., 2001), against desmogleins (DSG3 and DSG1, and occasionally desmocollin 3) resulting in acanthloysis (Kasperkiewicz et al., 2017). Classical pemphigus involves IgG autoantibodies, but mixed IgG/IgA and IgA forms are recognised (Hegazy et al., 2016; Toosi et al., 2016). Pemphigus vulgaris is characterised by anti-DSG3 antibodies with/without anti-DSG1, while patients with Pemphigus foliaceus exhibit anti-DSG1 antibodies.

Circulating pathogenic IgG autoantibodies from patients with pemphigus have been shown to disrupt keratinocyte monolayers in vitro (Di Zenzo et al., 2012) and to result in pemphigus-like disease lesions on passive transfer to neonatal mice in vivo (Anhalt et al., 1982), an effect that was dose-dependent. Furthermore, disease activity correlates with anti-DSG3 autoantibody titre (Ishii et al., 1997). Passive transplacental transfer of pathogenic autoantibodies from mothers with Pemphigus vulgaris has been reported to result in characteristic skin lesions which resolve spontaneously (Ruach et al., 1995). Accordingly, in pemphigus pathogenic immunoglobulin is both necessary and sufficient to induce disease. Substantiating the key role of B cells in the pathogenesis of pemphigus, B cell depletion using rituximab is clinically effective (Joly et al., 2007), with patients in complete remission characterised by near complete disappearance of desmoglein-specific circulating IgG+ B cells and serum anti-DSG antibodies (Colliou et al., 2013). Furthermore, Dsg-1 and Dsg-3 specific (i.e. antigen specific) B cells have been identified in pemphigus skin lesions, together with IgG⁺ plasma cells (Takahashi, 2017).

### Bullous Pemphigoid and Cicatricial Pemphigoid

Bullous pemphigoid is characterised immunologically by the presence of circulating IgG autoantibodies targeting the dermatoepidermal basement membrane zone, particularly the NC16a domain of the BP180 autoantigen (Diaz et al., 1990). Serum titres of anti-NC16a antibodies correlate with disease severity (Schmidt et al., 2000) and cause blister formation either directly or through complement fixation. Notably, circulating antigen-specific autoreactive plasmablasts and memory B cells specific for bullous pemphigoid autoantigens have been identified in patients with pemphigoid (Laszlo et al., 2010), with the latter able to differentiate into antibody secreting cells and produce anti-NC16a specific IgG antibodies in vitro (Leyendeckers et al., 2003). BAFF (B-cell activating factor belonging to the tumour necrosis factor (TNF) family), a key regulator of B cell survival and proliferation (including of autoreactive B cells) and thought to play a key role in the induction of autoimmune disease primarily or substantially mediated by B cells, is significantly elevated in serum of patients with bullous pemphigoid and decreases with treatment (Asashima et al., 2006). Moreover, BAFF has been identified on naïve and memory B cells in bullous pemphigoid but not healthy controls, where it may function as an autocrine factor promoting survival of autoreactive B cells (Qian et al., 2014). Further evidence for an environment conducive to B cell activation is the observation of elevated levels of circulating soluble CD40 ligand (sCD40L) in patients in bullous pemphigoid, particularly early at disease onset and in association with recurrences (Watanabe et al., 2007). Analogous findings have been observed in other autoimmune diseases such as SLE. The interaction between CD40 (on B cells and other antigen-presenting cells) and CD40L (appearing transiently on CD4⁺ T cells) is required for B cell differentiation and immunoglobulin class switching.

Substantiating a key pathogenic role for B cells in bullous pemphigoid, B cell depletion with rituximab has clinical efficacy in pemphigoid, including in recalcitrant and cicatricial cases (Li et al., 2011) with greater effect noted in IgG-dominant cases (Lamberts et al., 2018). Highlighting the importance of antibody secreting cell populations unaffected by rituximab therapy, persistence of autoreactive IgA-secreting plasmablasts/plasma cells has been described in association with refractory pemphigoid (He et al., 2015). Immunoapheresis, a therapeutic approach which removes immunoglobulins and immune complexes, has shown efficacy in severe/life threatening autoimmune bullous disease, including pemphigoid gestationis and pemphigus, in association with reduction in pathogenic immunoglobulin (Marker et al., 2011).

Autoimmune Alopecia (Autoimmune Hair Loss; Autoimmune Alopecia areata)

Alopecia areata (AA) is a common disorder characterised by acute onset of non-scarring hair loss, most frequently in patches affecting the scalp, but can involve hair loss of the entire scalp (alopecia totalis), facial hair (including eyebrows, eyelashes, beard), or loss of entire scalp and body hair (Alopecia universalis) (Islam et al., 2015). Alopecia areata is thought to reflect an organ-specific autoimmune disease of the hair follicle (Trueb and Dias, 2018).

Supporting an autoimmune basis for AA, patients often develop or have a history of other canonical autoimmune disease, including SLE, vitiligo, autoimmune thyroid disease, myaesthenia gravis and rheumatoid arthritis (Islam et al., 2015). While there is a recognised prominent CD8⁺ T cell-driven component directed against anagen-stage hair follicles (Guo et al., 2015), the pathobiology of AA is not fully understood. Plasma cells have been described in the peribulbar inflammatory infiltrate accompanying AA in patients (Elston et al., 1997; Ranki et al., 1984) and, using transmission electron microscopy, active plasma cells have been identified in acute AA (McElwee et al., 2013). Similar dermal observations together with hair follicle specific IgG on direct immunofluorescence have been noted in dogs exhibiting an AA homologue (Tobin et al., 2003). Antibodies to antigens selectively expressed in hair follicles of patients with AA have been identified (Tobin et al., 1994b). Circulating autoantibodies against hair follicle-specific keratins have also been described in C3H/HeJ mice with AA-like hair loss (Tobin et al., 1997). In contrast to the low levels of primarily IgM anti-hair follicle antibodies identified in normal individuals (Tobin et al., 1994a), those associated with AA are much higher in titre, not present in healthy individuals and of the IgG subclass, suggesting a class-switching as an important process in the immunopathogenesis of AA. Notably autoantibodies precede disease onset in the C3H/HeJ mouse model of AA, suggesting that the autoantibodies detected are not merely a secondary response to damage of hair follicles (Tobin, 2003). Notably, hair follicle autoantibody profile is modulated by topical therapy with diphencyprone used for AA, with very significant reductions in the titre of IgG anti-hair follicle antibodies in patients with complete and sustained hair regrowth, indicating that such autoantibody levels correlate with disease activity (Tobin et al., 2002). Supporting the pathogenic potential of such autoantibodies in AA, passive transfer of equine IgG fractions from a horse affected with AA-like hair loss to the anagen skin of wildtype C57BL mice disrupted hair regrowth around the site of injection, including up to 13 weeks post-injection, a finding not observed after injection of normal equine IgG (Tobin et al., 1998).

### Autoimmune Thyroid Disease (AITD), Including Graves' Disease and Hashimoto's Thyroiditis

AITD is an organ-specific autoimmune disorder characterised by breakdown of self-tolerance to thyroid antigens. Genome-wide association studies have revealed a role for genetic variants in B cell signalling molecules in the development of AITD (Burton et al., 2007), including FCRL3 (Chu et al., 2011b) and BACH2 involved in B cell tolerance, maturation and class switching (Muto et al., 2004). AITDLymphocyte.

Pathologically, AITD exhibits intense lymphocyte accumulation in the thyroid gland, including B cells at the time of diagnosis (notably in Hashimoto's thyroiditis) and production of anti-thyroid antibodies (Zha et al., 2014). Patients with recent-onset AITD display thyroid antigen-reactive B cells in the peripheral blood which are no longer anergic but express the activation marker, CD86, consistent with activation of these cells to drive autoantibody production (Smith et al., 2018).

Graves' disease is characterised by production of pathognomonic agonistic anti-thyrotropin receptor IgG autoantibodies (found in 80-100% of untreated patients) which mimic TSH and stimulate thyroid hormone overproduction and thyroid enlargement (Singh and Hershman, 2016). Patients with Graves' disease exhibit elevated transitional and pre-naïve mature B cells in peripheral blood, with levels positively correlating with those of free thyroxine (Van der Weerd et al., 2013). Consistent with a B cell-driven pathophysiological process and potentially contributing to the expansion of these B cell populations, the serum levels of BAFF (B lymphocyte activating factor)—a key factor promoting B cell autoantibody production by increasing B cell survival and proliferation—are raised in patients with Graves' disease and fall in response to methylprednisolone treatment (Vannucchi et al., 2012). Hyperthyroidism itself promotes plasma cytogenesis to increase plasma cells in the bone marrow (Bloise et al., 2014). B cell depletion using anti-mouse monoclonal CD20 antibody in a mouse immunisation model of model of Graves' disease is effective in suppressing anti-TSHR antibody generation and hyperthyroidism given before immunisation or 2 weeks later (Ueki et al., 2011). Mirroring this, rituximab has demonstrated efficacy clinically in Graves' orbitopathy (Salvi et al., 2013).

In Hashimoto's thyroiditis, B cells generate autoantibodies against thyroglobulin (>90% patients) and thyroid peroxidase which lead to apoptosis of thyroid follicular cells via antibody-dependent cell-mediated cytotoxicity. Plasma cell accumulation has been noted in thyroidectomy specimens from patients with Hashimoto's thyroiditis in association with foci of thyroid follicular destruction (Ben-Skowronek et al., 2013).

### Autoimmune Haemolytic Anaemia (AIHA)

AIHAs are autoimmune disorders characterised by pathogenic autoreactive antibodies against red blood cells leading to reduced red cell survival and anaemia (Garvey, 2008). Anti-red blood cell antibodies in these conditions are central to the destruction of red blood cells via either direct lysis (through complement activation) or antibody-dependent cytotoxicity (Barcellini, 2015).

In warm AIHA (wAIHA) this is mediated via macrophage FcγR recognition of IgG-coated red blood cells (polyclonal and of the IgG1 isotype, less so IgG3) and progressive membrane removal with ultimate formation of spherocytes which are trapped in splenic sinusoids and removed (LoBuglio et al., 1967). These autoantibodies are generated through the activation of autoreactive B cell clones (Fagiolo, 2004). Further evidence of a central role for B cells in AIHA comes from trial findings that B cell depletion with rituximab, in combination with glucocorticoid, shows efficacy over glucocorticoid alone in terms of both rate and duration of response (Birgens et al., 2013). Notably, active B cell responses are present in spleens of wAIHA patients, including large numbers of germinal centre B cells and plasmablasts/plasma cells which secrete anti-red blood cell antibodies (Mahevas et al., 2015). Patients with newly diagnosed wAIHA display an expansion of circulating GC-derived plasmablasts, more commonly IgG-secreting. Both splenic GC B cells and circulating plasmablasts reduce with corticosteroid therapy, indicating that the spleen is involved in both the destruction of red blood cells and the generation of autoreactive antibodies; as a corollary, splenectomy is associated with a durable response in many patients (Mahevas et al., 2015).

Cryoglobulinaemia, refers to the presence of cryoglobulins in the serum; these are immunoglobulins which precipitate in vitro below 37° C. and heterogeneous in composition (IgM, IgG or both). They result from mono- or poly-clonal B cell expansion, typically in association with lymphoproliferative disease, chronic infection or autoimmune disease (Ramos-Casals et al., 2012). In the setting of hepatitis C virus (HCV) infection, cryoglobulinaemia can occur as a B cell proliferation disorder and lead to systemic vasculitis through generation of monoclonal IgM which cross-reacts with immunoglobulins directed against HCV core proteins (Knight et al., 2010). Cryoglobulins are pathogenic through their ability to precipitate in the microcirculation and to induce immune-complex-mediated inflammatory injury. While treatment is focused on the underlying cause (e.g. antiviral therapy) combined with generalised immunosuppression, both plasma exchange and plasmapheresis (Payet et al., 2013) are effective in removing cryoglobulins in severe cases (Rockx and Clark, 2010). Highlighting the importance of B cells in the disease process, B cell depletion with rituximab is an effective therapeutic strategy in cryoglobulinaemic vasculitis (De Vita et al., 2012).

### Pernicious Anaemia (PA)

This refers to a megaloblastic anaemia due to impaired vitamin B12 absorption resulting from immune destruction of gastric parietal cells (which produce intrinsic factor required for B12 absorption) in the setting of atrophic gastritis (Bedeir et al., 2010). PA is characterised by circulating anti-parietal cell antibodies (^{˜}90% patients) of IgG, IgA and IgM isotypes and anti-intrinsic factor antibodies (^{˜}60%), the latter circulating IgG in class and specific markers for PA (Bizzaro and Antico, 2014). While autoimmune gastritis is thought of as a primarily T cell mediated disease, these autoantibodies are thought to contribute to the pathogenesis of PA. Specifically, parietal cell antibodies have been argued to promote destruction of gastric parietal cells based on preclinical studies administering these to rats (Tanaka and Glass, 1970), with demonstration of IgG antibodies on the surface of and within parietal cells suggesting access to the H⁺/K⁺ ATPase (Burman et al., 1992). Notably parietal cell autoantibodies can predict development of overt atrophic gastritis (Tozzoli et al., 2010). In the case of intrinsic factor, while circulating autoantibodies are IgG in class, those secreted into gastric juice are IgA and thought to contribute to progression of autoimmune gastritis to PA (Osborne and Sobczynska-Malefora, 2015). Intrinsic factor antibodies can block the binding of cobalamin to intrinsic factor, or to block the binding of the intrinsic factor-cobalamin complex to its receptor in the ileum (Rowley and Whittingham, 2015). Recently, an increase in IgG4+ plasma cells has recently been identified in gastric mucosa of PA patients and not observed in other types of gastritis, suggesting specific involvement of these cells in the disease process (Bedeir et al., 2010).

### Myaesthenia Gravis (MG)

In MG, IgG autoantibodies directed against the nicotinic acetylcholine receptor (in ^{˜}85% of patients) or other synaptic antigens (muscle-specific kinase and low-density lipoprotein receptor-related protein 4) present at the neuromuscular junction result in skeletal muscle weakness. The autoantibodies affect the function of these antigens to induce disease via multiple mechanisms, including complement-mediated membrane destruction (Engel and Arahata, 1987), antigenic modulation (e.g. cross-linking by bivalent IgG1 and IgG3 to result in internalisation of AChR to reduce the available cell surface pool) (Drachman et al., 1978), ligand binding site competition (e.g. with ACh) (Drachman et al., 1982) and potentially steric hindrance (Huijbers et al., 2014).

Mirroring early observations of the ability of patient-derived immunoglobulin fraction of serum to induce disease in mice (Toyka et al., 1975), direct isolation and analysis of the anti-AChR antibody repertoire from peripheral memory B cells of patients with MG has identified pathogenic antibody (B12L) which induces a myasthenic phenotype in rats upon single dose passive transfer, with evidence of dose-dependency (Makino et al., 2017).

In addition to producing pathogenic immunoglobulin, multiple lines of evidence confirm the fundamental importance of B cells in the immunopathogenesis of MG. A deformed naïve and memory B cell repertoire has been identified consistent with defective tolerance checkpoints in the naïve compartment (Vander Heiden et al., 2017). Other observations indicate increased frequency of newly emigrant or transitional B cells and mature naïve B cells with autoreactive B cell receptors, further indicating defective central (i.e. bone marrow) tolerance mechanisms in MG (Lee et al., 2016a). These, coupled with the presence of additional autoantibody specificities and high frequency of a second autoimmune disease in such patients, highlight the importance of dysregulated B cell self-tolerance in the pathogenesis of MG.

Studies of thymic populations from patients with MG have revealed B cells organised in germinal centres which are activated (Leprince et al., 1990), with thymic lymphocytes able to synthesis anti-AChR antibody (Vincent et al., 1978). As a corollary, thymectomy is associated with clinical improvement associated with a fall in autoantibody titre (Vincent et al., 1983; Wolfe et al., 2016). In addition to thymus, pathogenic antibody secreting cells have been identified in lymph nodes (Fujii et al., 1985a) and the bone marrow of patients with MG (Fujii et al., 1985b). Patients with MG have been observed to feature an expanded circulating plasmablast/plasma cell pool (Kohler et al., 2013).

### Neuromyelitis Optica (NMO)

NMO is a demyelinating disorder of the central nervous system (CNS) typically presenting with recurrent episodes of optic neuritis and transverse myelitis. The majority (^{˜}75%) of patients exhibit IgG autoantibodies against glial aquaporin-4 (AQP4) water channels (Bennett et al., 2015). Intracerebral co-injection of IgG from AQP4 positive NMO patients with human complement into mice recapitulates key aspects of NMO histology, including loss of AQP4 expression, glial cell oedema, breakdown of myelin, cerebral oedema and neuronal cell death (Saadoun et al., 2010). Critically supporting a direct pathogenic role for these autoantibodies in mediating CNS injury, these features were not observed when IgG from non-NMO patients was used, or injection of IgG from NMO patients into AQP4-null mice (Saadoun et al., 2010). Plasmablasts are expanded in the peripheral blood of patients with NMO, capable of producing anti-AQP4 autoantibodies, with IgG plasmablasts enriched in cerebrospinal fluid (CSF) lymphocytes during NMO relapses (Chihara et al., 2013). Furthermore, IgG plasmablasts from peripheral blood and CSF of patients with NMO exhibit high frequencies of mutations in complementarity-determining regions (CDR) consistent with a post-germinal centre lineage and share CDR sequences suggesting migration of plasmablasts from periphery to the CSF to promote local autoantibody production (Chihara et al., 2013). Indeed, peripheral blood plasmablasts have been shown to be the primary producers of anti-AQP4 antibodies in the blood, further increased during relapses and promoted by IL-6 whose levels are increased in NMO (Chihara et al., 2011). Depletion of B cells using rituximab reduces NMO relapse frequency in patients (Damato et al., 2016).

### Autoimmune Epilepsy Syndromes and Autoimmune Encephalitis

The autoimmune epilepsy syndromes are immune-mediated disorders characterised by recurrent, uncontrolled seizures which are often anti-epileptic drug resistant (Britton, 2016). While seizures are a recognised feature of autoimmune encephalitis and multifocal paraneoplastic disorders, they are increasingly recognised in the absence of typical syndromic features of encephalitis, i.e. as a distinct entity (Britton, 2016).

Autoantibodies against neural antigens such as voltage-gated potassium channel (VGKC) complex proteins, glycine receptors, glutamate/AMPA receptor subtype 3, glutamic acid decarboxylase (GAD), N-methyl-D-aspartate (NMDA) receptors (NMDAR), collapsin response-mediator protein 5 and ganglionic acetylcholine receptor are well-described in cohorts of patients with epilepsy including in those newly diagnosed and frequently resistant to conventional anti-epileptic drugs (Brenner et al., 2013; Ganor et al., 2005; McKnight et al., 2005; Quek et al., 2012). Supporting an immune basis for the manifestations, such cases have been reported to respond well to immunotherapy including IV immunoglobulin and plasmapheresis (Quek et al., 2012). Clear correlation between autoantibodies and clinical seizures have been identified such as for GABA in limbic encephalitis with seizures (Lancaster et al., 2010) and NMDA in the context of anti-NMDA receptor encephalitis (Dalmau et al., 2008).

A large body of evidence supports a key role for B cells and the pathogenicity of autoantibodies in autoimmune encephalitis. The neuronal pathology of autoimmune encephalitis includes evidence of immunoglobulin on the surface of neurons (e.g. anti-VGKC-complex encephalitis), together with infiltration of CD20+ B cells and CD138+ plasma cells, supporting a B cell-mediated disease mechanism, particularly in those encephalitides with antibodies directed against surface antigens (Bien et al., 2012). Patients with treatment-naive autoimmune NMDAR encephalitis exhibit intrathecal (i.e. within CSF) B cell and plasma cell accumulation and intrathecal anti-NMDAR IgG antibody production (Malviya et al., 2017). Moreover, both intrathecal B cell and plasma cell accumulation correlate well with disease course and reflect response to immunotherapy (Malviya et al., 2017).

Hippocampal neurons cultured with CSF or purified IgG containing autoantibodies against NMDA from patients with NMDAR encephalitis results in reduced surface NMDAR cluster expression in a titre-dependent manner via cross-linking and internalisation of the receptors (Hughes et al., 2010). Consistent with an impact on function, patients' antibodies selectively reduce NMDAR currents of cultured rat hippocampal neurons (Hughes et al., 2010).

Passive transfer of cerebrospinal fluid (CSF) from patients with NMDAR encephalitis into the cerebral ventricles of wildtype C57BL6/J mice results in progressive memory deficits, anhedonic and depressive like behavioural changes which worsen over 14 days and resolve upon discontinuation of infusion, effects not seen with control CSF (Planaguma et al., 2015). Histologically these clinical features were accompanied by progressive elevation in brain-bound anti-NMDAR antibodies, largely in the hippocampus, and reduced surface density of NMDAR (Planaguma et al., 2015). Conversely, reversibility and recovery were associated with a fall in brain-bound antibody levels and recovery of NMDAR concentration. Further implicating NMDAR encephalitis as a humorally driven autoimmune disease, single recombinant human NMDAR-specific monoclonal antibody reconstructed from patient-derived clonally expanded intrathecal plasma cells is sufficient to recapitulate key features of NMDAR encephalitis in vitro and in vivo (Malviya et al., 2017).

Clinically, autoantibody levels in patients with autoimmune encephalitis (such as anti-NMDA receptor encephalitis) correlate with neurological outcome, with antibody levels in CSF more closely correlated with relapses than levels in serum (Gresa-Arribas et al., 2014).

Further supporting a pathogenic role for autoantibodies in patients with autoimmune encephalitis, removal of antibodies using immunoadsorption accelerates recovery in patients with antibodies against leucine-rich, glioma inactivated 1 (LG1), contactin-associated protein-2 (CASPR2) or NMDAR (Dogan Onugoren et al., 2016). Similarly, plasma exchange resulted in marked improvement in seizure frequency in a patient with anti-GAD antibody-related epilepsy in conjunction with substantial reduction of autoantibody burden (Farooqi et al., 2015). Substantiating a specific role for B cells, B cell depletion with rituximab has reported efficacy in refractory autoimmune encephalitis (Lee et al., 2016b; Strippel et al., 2017). Furthermore, plasma cell depletion with the proteasome inhibitor bortezomib has been reported to be effective in a case of extremely severe refractory anti-NMDAR encephalitis (Sveinsson et al., 2017).

### Autoimmune Hepatitis (AIH)

Autoimmune hepatitis is an immune-mediated liver disorder characterised by autoantibodies, elevated IgG levels and hepatitis. AIH is associated with a striking plasma cell infiltration/accumulation in lobular and periportal hepatic regions as a hallmark feature present in ^{˜}90-100% of cases, including with acute presentations (Fujiwara et al., 2008; Nguyen Canh et al., 2017). Flow cytometry analysis of peripheral blood of patients with new-onset AIH indicates an expansion in circulating B cells, activated B cells and plasma cells compared to controls. Notably such AIH patients also exhibit an increase in circulating T follicular helper cells, key regulators of humoral immunity through their promotion of the germinal centre response (Ma et al., 2014). Moreover, significantly increased serum IL-21—a key cytokine produced by T follicular helper cells which acts to promote B cell differentiation in antibody-secreting cells (Bryant et al., 2007)—is present in patients with AIH compared to healthy controls and positively correlates with serum levels of IgG, IgA and IgM (Ma et al., 2014).

AIH is associated with characteristic autoantibodies, with type 1 AIH exhibiting anti-nuclear (ANA) and/or anti-smooth muscle (SMA) autoantibodies, while type 2 AIH features anti-liver kidney microsomal type 1 and/or anti-liver cytosol type 1 antibodies (Liberal et al., 2013). Notably anti-ANA and SMA titres reduce or disappear with effective therapy in type 1 AIH (Liberal et al., 2013). While the precise pathogenic role of these autoantibodies is debated, the frequency of detection of these antibodies, prominence of plasma cells histologically and correlation of serum IgG and autoantibody levels/type (including anti-liver specific) with disease activity (including histology, aminotransferase levels and disease severity) strongly support a humoral/antibody-mediated component to AIH (Jensen et al., 1978; Ma et al., 2002; Sebode et al., 2018). Furthermore, isolated hepatocytes from patients with AIH are covered with surface immunoglobulin of the IgG subclass which is associated with greater susceptibility to antibody-dependent cell-mediated cytotoxicity in vitro (Vergani et al., 1987).

Supporting a role for B cells in AIH, serum levels of BAFF are elevated in AIH, positively correlate with markers of liver injury and dysfunction (aminotransferases and bilirubin) and fall in response to corticosteroid treatment (Migita et al., 2007). B cell depletion with anti-CD20 antibody dramatically reduces liver inflammation in and alanine aminotransferase levels in a mouse model of AIH (Beland et al., 2015). Similarly, patients with treatment refractory AIH have been shown to respond to B cell depletion using the anti-CD20 monoclonal antibody, rituximab (Burak et al., 2013).

### Chronic Autoimmune Urticaria (Chronic Spontaneous Urticaria, CSU)

Chronic autoimmune urticaria or chronic idiopathic urticaria, now termed chronic spontaneous urticaria (CSU) is a skin disorder associated with mast cell and basophil degranulation with associated release of histamine, leukotrienes, prostaglandins and other substances resulting in recurrent weals (hives), angiooedema or both for over 6 weeks (de Montjoye et al., 2018; Kolkhir et al., 2017). Activation of these cells is thought to be autoimmune mediated involving either a type 1 or type II hypersensitivity response, the latter referring to autoantibodies binding to antigens on target cells. IgG autoantibodies to IgE and FcεRI (the high affinity receptor for IgE present on mast cells and basophils) are well-described in patients with CSU and in the case of the latter can promote receptor cross-linking and histamine release (Fiebiger et al., 1995; Hide et al., 1993; Sabroe et al., 2002; Sun et al., 2014; Tong et al., 1997).

Supporting a pathogenic functional role for these IgG autoantibodies (including anti-FcεRI) is the finding that they can induce histamine release from healthy skin mast cells and basophils (Grattan et al., 1991; Niimi et al., 1996). In addition, IgG antibody can promote complement activation following cross-linking of FcεRI to generate C5a which further enhances target cell degranulation (e.g. basophil) and histamine release (Kikuchi and Kaplan, 2002). In addition, both heterologous and autologous injection of IgG anti-FcεRI containing serum result in a weal and flare response (Kolkhir et al., 2017). Notably patients with positive autologous serum skin tests exhibit greater clinical severity (Caproni et al., 2004), longer duration and higher requirement for antihistamines (Staubach et al., 2006).

Removal of pathogenic autoantibodies using plasmapheresis has induced marked clinical responses in patients with severe unremitting CSU, in parallel with reduction in both serum IgG and in vitro measure of histamine-releasing activity (of patient serum on mixed leucocytes of healthy donors) (Grattan et al., 1992). Notably, the efficacy of omalizumab—a monoclonal antibody which selectively binds human IgE—is thought to in part be mediated through downregulation of FcεRI density on mast cells and basophils (MacGlashan et al., 1997; Saini et al., 1999) thereby preventing IgG autoantibody-mediated cross-linking of adjacent receptors (Kaplan et al., 2017).

The source of these functional IgG autoantibodies are thought to be peripheral B cells (Chakravarty et al., 2011). CSU has been shown to be associated with polyclonal B cell activation, including production of other autoantibodies and increased serum IgE levels, together with enhanced B cell proliferation (Kessel et al., 2010; Toubi et al., 2000). Furthermore, serum levels of BAFF, a crucial B cell survival, activation and maturation signal, are elevated in patients with CSU and associate with disease severity (Kessel et al., 2012). B cell depletion using rituximab has reported remarkable clinical efficacy in refractory CSU and is associated with negative basophil histamine release assay (Chakravarty et al., 2011; Combalia et al., 2018; Steinweg and Gaspari, 2015).

### Linear IgA Disease (LAD)

Linear IgA disease (LAD) is a chronic, acquired, autoimmune subepidermal bullous skin disease characterised by IgA autoantibody deposition at the dermal-epidermal junction and/or by circulating IgA autoantibodies directed against heterogeneous basement membrane zone antigens (Kasperkiewicz et al., 2010; Kirtschig and Wojnarowska, 1999; Utsunomiya et al., 2017).

Supporting a pathogenic role for IgA autoantibodies in LAD, immunoadsorption using a tryptophan-based immunoadsorper resulted in striking clinical improvement in LAD together with a reduction in total IgA (Kasperkiewicz et al., 2010). Supporting this are pre-clinical experiments demonstrating that passive transfer of IgA mouse monoclonal antibodies against a linear IgA antigen to SCID mice with human skin grafts can result in consistent IgA deposition at the basement membrane zone, neutrophil infiltration and basement membrane zone vesiculation (Zone et al., 2004).

Highlighting a role for B cells in LAD, B cell depletion with rituximab has evidenced clinical efficacy in severe/recalcitrant cases (Pinard et al., 2019).

### IgA Nephropathy

In IgA nephropathy, increased presence of poorly O-galactosylated IgA1 glycoforms in the serum, subsequent O-glycan specific IgA and IgG autoantibody production (Suzuki et al., 2009) and resultant formation and deposition of IgA1 immune complex in the glomerular mesangium serve to initiate renal injury and glomerulonephritis which can progress to renal failure (Lai et al., 2016; Tomana et al., 1999). Thus IgA or IgA immune complex deposition are regarded as fundamental causal factors in IgA nephropathy (Suzuki and Tomino, 2008).

Serum levels of IgG and IgA autoantibodies (recognising galactose-deficient IgA1 as an autoantigen) are significantly associated with progression of IgA nephropathy (dialysis/death) (Berthoux et al., 2012). Notably the serum concentration of autoantigen (galactose-deficient-IgA1) and IgG autoantibody correlate (Placzek et al., 2018). As a corollary, serum levels of galactose-deficient IgA1 (autoantigen) driving pathogenic autoantibody production in IgA nephropathy independently associate with higher risk of deterioration in renal function (Zhao et al., 2012).

Further evidence supporting the importance of autoantibodies and the targeting of the specific cells producing these rather than generalised B cell depletion comes from a trial investigating the therapeutic potential of rituximab in IgA nephropathy (Lafayette et al., 2017). B cell depletion using rituximab in patients with IgA nephropathy with significant proteinuria and renal impairment failed to impact on serum levels of galactose-deficient IgA1 and anti-galactose-deficient IgA1 antibodies and, accordingly, did not favourably affect renal function (Lafayette et al., 2017).

Patients with IgA nephropathy have an expansion in bone marrow IgA plasma cells compared to controls, particularly subclass IgA1, suggesting that the bone marrow is the primary site of production of IgA deposited in the kidney mesangium in IgA nephropathy (van den Wall Bake et al., 1988). Furthermore, a positive correlation between bone marrow IgA plasma cells and serum IgA has been identified (Harper et al., 1994). Supporting these findings suggesting mephritogenic IgA1 production in bone marrow, bone marrow transplantation has been reported to result in complete remission of IgA nephropathy (Iwata et al., 2006).

Patients with IgA nephropathy also feature a higher frequency of circulating memory B cells, activated B cells, T follicular helper cells and plasma cells (Sun et al., 2015; Wang et al., 2014). Notably higher circulating levels of memory and activated B cells and T follicular helper cells correlated with more advanced disease (judged by proteinuria) (Sun et al., 2015). Higher serum levels of APRIL (a proliferation-inducing ligand, also known as TNFSF13), which mediates class-switching largely for IgA and is critical for survival of bone marrow and mucosal plasma cells, associate with worse prognosis of IgA nephropathy (Han et al., 2016). A role for APRIL in genetic susceptibility to IgA nephropathy is also supported by genome-wide association studies (Yu et al., 2011). Furthermore, a role for aberrant expression of APRIL in tonsillar germinal centre B cells in IgA nephropathy has been found, correlating with greater proteinuria and suggesting a role for tonsillar B cells underlying the response of IgA nephropathy to tonsillectomy (Muto et al., 2017).

### Vitiligo

Vitiligo is an acquired chronic depigmenting disease resulting from selective melanocyte destruction (Ezzedine et al., 2015).

Patients with vitiligo frequently exhibit autoantibodies at levels higher than controls, including anti-thyroperoxidase, anti-thyroglobulin, antinuclear, anti-gastric parietal cell and anti-adrenal antibodies (Liu and Huang, 2018), some of which correlate with clinical vitiligo activity (Colucci et al., 2014). In comparison to controls, vitiligo is associated with elevated total IgG, IgG1 and IgG2 and melanocyte-reactive antibodies (Li et al., 2016b). The latter are most frequently directed against pigment cell antigens (Cui et al., 1992), including melanin-concentrating hormone receptor 1 (Kemp et al., 2002). Melanocyte death in vitiligo has been proposed to reflect apoptosis and is promoted in vitro by serum IgG from vitiligo patients (Ruiz-Arguelles et al., 2007). Notably IgG (and C3) deposits have been observed in the basement membrane zone of lesional skin. Furthermore, binding of IgG from vitiligo patients to cultured melanocytes increases with disease extent and activity, with further correlation of vitiligo activity to levels of anti-melanocyte IgA (Kemp et al., 2007).

While there is debate regarded whether the presence of autoantibodies in vitligo reflects a primary cause or consequence of the disease, it is clear that vitiligo autoantibodies possess the capacity to result in pigment cell injury via multiple effector mechanisms, including antibody-dependent cellular cytotoxicity and complement-mediated cell damage in vitro (Cui et al., 1993; Norris et al., 1988).

MCHR function-blocking autoantibodies have also been identified in vitiligo patients, which would be expected to interfere with normal melanocyte function (Gottumukkala et al., 2006). In addition to the role of MCHR1 as a B cell autoantigen, the importance of B cells is further suggested in vitiligo through identification of Bcl-2 positive infiltrates in close juxtaposition to areas of depigmentation (Ruiz-Arguelles et al., 2007). Vitiligo has also been reported to respond to B cell depletion with monoclonal antibody to CD20 (Ruiz-Arguelles et al., 2013).

### Primary Biliary Cirrhosis (PBC)

Primary biliary cirrhosis (PBC), also known as primary biliary cholangitis, is a chronic cholestatic liver disorder characterised pathologically by progressive small intrahepatic bile duct destruction with associated portal inflammation, fibrosis and risk of progression to cirrhosis, and serologically (>95%) by anti-mitochondrial antibody (AMA) and often an elevated serum IgM (Carey et al., 2015). Notably, autoantibodies (e.g. anti-centromere) are strongly associated with risk of progression to cirrhosis and portal hypertension (Nakamura, 2014).

While T cells have been reported to constitute the majority of cellular infiltrate in early PBC, B cells/plasma cells are also identified (Tsuneyama et al., 2017). Specifically, formation of follicle-like aggregations of plasma cells expressing IgG and IgM around intrahepatic ducts have been noted in patients with PBC, further correlating with higher titres of AMA (Takahashi et al., 2012). The finding of oligoclonal B cell proliferation and accumulation of somatic mutations in liver portal areas from patients with PBC is consistent with antigen-driven B cell responses (Sugimura et al., 2003). A sustained rigorous B cell response in PBC has also been suggested through the finding of high levels of autoantigen-specific peripheral plasmablasts (to the pyruvate dehydrogenase complex autoantigen PDC-E2) consistent with ongoing activation of autoreactive B cells (Zhang et al., 2014). Notably, newly diagnosed patients with PBC exhibit elevated numbers of circulating T follicular helper cells and plasma cells, with both correlating positively with each other, as well as with levels of serum AMA and IgM (Wang et al., 2015). Rituximab has been reported to reduce serum total IgG, IgA and IgM, in addition to AMA IgA and IgM in patients with PBC and an incomplete response to ursodeoxycholic acid (Tsuda et al., 2012), in addition to a limited but discernible favourable effect on alkaline phosphatase and pruritus (Myers et al., 2013).

### Primary Sclerosing Cholangitis (PSC)

PSC is a chronic liver disorder characterised by multifocal biliary strictures and high risk of cholangiocarcinoma, together with strong association with inflammatory bowel disease (Karlsen et al., 2017). A large number of autoantibodies have been detected in patients with PSC, but generally of low specificity, including pANCA, ANA, SMA and anti-biliary epithelial cell (Hov et al., 2008). Notably and consistent with the known physiologically dominant role for secreted IgA in bile, the presence of autoreactive IgA against biliary epithelial cells correlates with faster clinical progression of PSC (to death/liver transplantation) (Berglin et al., 2013).

Functional IgA, IgM and IgG antibody secreting cells have been identified in PSC liver explants (Chung et al., 2016). Notably, the majority of these cells are plasmablasts rather than plasma cells (Chung et al., 2017). Alterations in the peripheral circulating T follicular helper cell compartment, a key facilitator of antibody responses, have been identified in PSC (Adam et al., 2018). Supporting a role for shared liver and gut adaptive immune response in PSC associated with inflammatory bowel disease, B cells of common clonal origin have been identified in both tissues together with evidence of higher somatic hypermutation consistent with (same) antigen-driven activation (Chung et al., 2018).

### Autoimmune Thrombocytopenic Purpura (Immune Thrombocytopenia; Adult Immune Thrombocytopenia)

Immune thrombocytopenia (ITP) is a disorder characterised by acquired thrombocytopenia (low platelet count) driven by immune recognition of platelet autoantigens and ensuing destruction of platelets.

Highlighting the importance of humoral immune mechanisms were early studies revealing that infusion of serum from patients with ITP to healthy volunteers resulted in profound thrombocytopenia, that this was dose-dependent, that the humoral factor could be adsorbed by platelets and in the IgG fraction (Harrington et al., 1951; Karpatkin and Siskind, 1969; Shulman et al., 1965). In addition to IgG autoantibodies against platelet glycoprotein (GP)IIb/IIIa, IgA and IgM anti-platelet autoantibodies have been identified (He et al., 1994), as well as against other platelet surface proteins such as GPIb/IX, with a high degree of specificity for ITP (McMillan et al., 2003). These autoantibodies result in antibody-dependent platelet phagocytosis seen in vitro (Tsubakio et al., 1983) and in vivo by splenic macrophages and peripheral neutrophils (Firkin et al., 1969; Handin and Stossel, 1974). Notably the amount of platelet-associated IgG inversely correlates with the platelet count (Tsubakio et al., 1983).

In addition to promoting platelet destruction, autoantibodies have also been demonstrated to directly affect bone marrow megakaryocyte maturation (Nugent et al., 2009). Both GPIIb/IIIa and GPIb/IX are expressed on megakaryocytes, with autoantibodies found binding to these in ITP (McMillan et al., 1978). Furthermore, plasma from patients with ITP suppresses megakaryocyte production and maturation in vitro, an effect ameliorated through adsorption of autoantibody with immobilised antigen and also seen with patient IgG but not control IgG (McMillan et al., 2004).

Splenectomy samples from patients with ITP show marked follicular hyperplasia with germinal centre formation and increased plasma cells consistent with an ongoing active B cell response in ITP (Audia et al., 2011). Notably, frequency of splenic T follicular helper cells is higher in ITP compared to controls, with further expansions in splenic pre-germinal centre B cell, germinal centre B cell (in addition to plasma cells) also identified, and all correlating positively with percentage of T follicular helper cells (Audia et al., 2014). B cell depletion with rituximab is effective in improving platelet count in ^{˜}60% of patients with ITP, with patients in whom autoantibody is persistent more frequently failing to demonstrate a clinical response (Arnold et al., 2017; Khellaf et al., 2014).

Highlighting an important role for long-lived plasma cells as a substrate for ongoing generation of pathogenic autoantibodies mediating platelet destruction and reduced production, patients who are refractory to B cell depletion with rituximab display autoreactive anti-GpIIb/IIIa plasma cells in spleen expressing a long-lived genetic programme (Mahevas et al., 2013).

### Autoimmune Addison's Disease (AAD)

AAD is a rare autoimmune endocrinopathy characterised by an aberrant immune destructive response against adrenal cortical steroid producing cells (Mitchell and Pearce, 2012).

A major autoantigen in AAD is steroid 21-hydroxylase with the majority (>80%) of patients exhibiting autoantibodies against this (Dalin et al., 2017), with sera from patients with AAD reacting with the zona glomerulosa of the adrenal cortex (Winqvist et al., 1992). Anti-adrenal antibodies are predictive of progression to overt disease or subclinical adrenal insufficiency in patients with other autoimmune disorders (Betterle et al., 1997). Notably, levels of adrenal autoantibodies correlate with severity of adrenal dysfunction, suggesting association with the destructive phase of autoimmune adrenalitis. Conversely, patients exhibiting biochemical remission of adrenal dysfunction, including in response to corticosteroid therapy, also display loss of adrenal cortex autoantibody and 21-hydroxylase autoantibody (De Bellis et al., 2001; Laureti et al., 1998). While it is unclear whether these autoantibodies are directly pathogenic (particularly given their intracellular target), organ-specific reactive antibodies have been demonstrated from AAD sera (Khoury et al., 1981).

Histologically, AAD is characterised by a diffuse inflammatory infiltrate, including plasma cells (Bratland and Husebye, 2011).

Genetic support for an important role for B cells in the susceptibility to AAD has come from the identification of BACH2 as a major risk locus (Eriksson et al., 2016; Pazderska et al., 2016). BACH2 encodes a transcriptional repressor which is required for class switch recombination and somatic hypermutation in B cells through regulation of the B cell gene regulatory network (Muto et al., 2010; Muto et al., 2004). Administration of rituximab to induce B cell depletion in AAD has reported efficacy in a new-onset case, with evidence of sustained improvement in cortisol and aldosterone (Pearce et al., 2012).

### Multiple Sclerosis (MS)

MS is an inflammatory demyelinating disorder of the central nervous system (CNS).

While MS is typically conceptualised as a CD4 Th1/Th17 T cell-mediated disorder, largely based on findings using the experimental autoimmune encephalomyelitis (EAE) model, T cell-specific therapies have not demonstrated clear efficacy in relapsing-remitting MS (Baker et al., 2017). In contrast, many active MS immunomodulatory and disease-modifying therapies are recognised to affect the B cell compartment and/or serve to deplete memory B cells, either physically or functionally (Baker et al., 2017; Longbrake and Cross, 2016).

The most well-recognised and persistent immunodiagnostic abnormality in MS—the presence of oligoclonal bands in cerebrospinal fluid (CSF) typically of IgG isotype (but also IgM)—is a product of B lineage cells (Krumbholz et al., 2012). Notably clonal IgG in CSF is stable over time, consistent with local production from resident long-lived plasma cells or antibody secreting cells maturing from memory B cells (Eggers et al., 2017). That anti-CD20 therapy reduces CSF B cells with no significant impact on oligoclonal bands suggests a substantial role for long-lived plasma cells in oligoclonal band production (Cross et al., 2006). Correlation of immunoglobulin proteomes in CSF samples has revealed strong overlap with transcriptome of CSF B cells highlighting the latter as the source (Obermeier et al., 2008). The majority of B cells in the CSF of patients with MS are memory B cells and short-lived plasmablasts, with the latter representing the main source for intrathecal IgG synthesis and correlating with parenchymal inflammation revealed by MRI (Cepok et al., 2005), with evidence of greater involvement in acute inflammation associated with relapsing-remitting MS (Kuenz et al., 2008).

Pathologically, organised ectopic tertiary lymph node-like structures with germinal centres are present in the cerebral meninges in MS (Serafini et al., 2004). As with parenchymal lesions, B cell clones in meningeal aggregates largely use IgG (^{˜}90%, remainder IgM) (Lovato et al., 2011). Moreover, antigen experienced B cell clones are shared between these meningeal aggregates and corresponding parenchymal lesions (Lovato et al., 2011). In addition, flow cytometry with deep immune repertoire sequencing of peripheral blood and CSF B cells indicate that peripheral class-switched B cells, including memory B cells, have a connection to the CNS compartment (Palanichamy et al., 2014). Notably memory B cells have recently been demonstrated to promote autoproliferation of Th1 brain-homing autoreactive CD4⁺ T cells in MS (Jelcic et al., 2018).

The best characterised autoantigen in MS is myelin oligodendrocyte glycoprotein (MOG), the target of autoantibodies in EAE and against which antibodies are identified in ^{˜}20% children but relatively few adults with demyelinating disorders (Krumbholz et al., 2012; Mayer and Meinl, 2012). Evidence supporting a role for pathogenic autoantibody in MS includes the efficacy of plasma exchange in some patients (Keegan et al., 2005) and the presence of complement-dependent demyelinating/axopathic autoantibodies in a subset of patients with MS (Elliott et al., 2012). Other autoantibodies have been identified against axoglial proteins around the node of Ranvier including autoantibodies against contactin-2 and neurofascin, with evidence of axonal injury evident using in vivo models when transferred with MOG-specific encephalitogenic T cells and inhibition of axonal conduction when used with hippocampal slices in vitro (Mathey et al., 2007).

Substantiating a key role for B cells in relapsing-remitting MS, B cell depletion using the chimeric anti-CD20 antibody rituximab reduces both inflammatory brain lesions and clinical relapses (Hauser et al., 2008). Similar unequivocally positive efficacy findings have been observed with use of other CD20 depleting agents such as ocrelizumab (humanised monoclonal anti-CD20 antibody) in relapsing MS (Hauser et al., 2017) and primary progressive MS (Montalban et al., 2017).

### Type 1 Diabetes Mellitus (T1DM)

T1DM is an autoimmune disorder characterised by immune-mediated destruction of the pancreatic islet β cells. While the major cellular effectors of islet β cell destruction are generally considered as islet antigen-reactive T cells, a large body of evidence implicates B cells in this process and the pathogenesis of the disease (Smith et al., 2017).

The non-obese diabetic (NOD) mouse model of autoimmune diabetes exhibits an autoimmune insulitis. B cell deficient NOD mice exhibit suppression of insulitis, preservation of islet β cell function and protection against diabetes compared to NOD mice, indicating that B cells are essential for the development of diabetes in this model (Akashi et al., 1997; Noorchashm et al., 1997). Similar findings have been observed through use of anti-CD20 mediated B cell depletion, including reversal of established hyperglycaemia in a significant proportion of mice (Hu et al., 2007). Substantiating an important role for B cells in the pathogenesis of human T1DM, B cell depletion using rituximab results in partial preservation of islet β cell function in patients with newly diagnosed T1DM at 1 year (Pescovitz et al., 2009).

Studies with NOD mice suggest that islet autoantigen presentation by B cells to T cells is an important component of their pathogenic effect (Marino et al., 2012; Serreze et al., 1998). Alterations in peripheral blood B cell subsets have been identified in T1DM patients, including reduction in transitional B cells and an increase in plasmablast numbers (Parackova et al., 2017). In addition, circulating activated T follicular helper cells are increased in children with newly diagnosed T1DM and autoantibody positive at risk children (Viisanen et al., 2017).

The preclinical phase of T1DM is characterised by the presence if circulating islet autoantibodies, such as glutamic acid decarboxylase 65 (GAD65) and insulinoma antigen 2 (IA2) autoantibodies. The majority of children genetically at risk for T1DM with multiple islet autoantibody serocoversion subsequently progress to clinical diabetes (Ziegler et al., 2013). While these autoantibodies are predictive of development of T1DM, their precise pathogenic role is debated. Supporting evidence for their pathogenicity comes from studies in NOD mice where elimination of maternal transmission of autoantibodies from prediabetic NOD mice protects progeny from development of diabetes (Greeley et al., 2002). Notably, NOD mice deficient in activating Fc receptors for IgG (FcγR) are protected from spontaneous onset of T1DM (Inoue et al., 2007).

### Coeliac Disease and Dermatitis Herpetiformis

Coeliac disease is a chronic immune-mediated enteropathy against dietary gluten in genetically predisposed individuals (Lindfors et al., 2019). Adaptive immune responses play a key role in the pathogenesis of coeliac disease characterised by both antibody production towards wheat gliadin (IgA and IgG) and tissue transglutaminsase 2 enzyme (TG2) (IgA isotype), together with gluten-specific CD4⁺ T cell responses in the small intestine (van de Wal et al., 1998). The finding of TG2 as the primary autoantigen present in endomysium and the target for endomysial antibodies secreted by specific B cells (Dieterich et al., 1997) forms the basis of the primary coeliac antibody test used to support a diagnosis of coeliac disease with ^{˜}90-100% sensitivity/specificity (Rostom et al., 2005).

Multiple potentially pathogenic effects have been ascribed to coeliac disease autoantibodies (Caja et al., 2011) including of the IgA subclass, such as: interference with intestinal epithelial cell differentiation (Halttunen and Maki, 1999); promotion of retrotranscytosis of gliadin peptides to enable their entry into the intestinal muscosa to trigger inflammation (Matysiak-Budnik et al., 2008); increased intestinal permeability and induction of monocyte activation (Zanoni et al., 2006); and inhibition of angiogenesis via targeting of blood vessel TG2 in the lamina propria (Myrsky et al., 2008).

B cells specific for gluten and TG2 have been proposed to act as antigen-presenting cells to gluten-specific CD4⁺ T cells, with HLA-deamidated gluten peptide-T cell receptor interaction resulting in activation of both T and B cell, the latter differentiating into plasma cells with ensuing production of antibodies targeting gliadin and endogenous TG2 (du Pre and Sollid, 2015; Sollid, 2017).

While genetic association studies highlight a key role for CD4⁺ T cells in the pathogenesis of coeliac disease, integrative systems biology approaches have highlighted a significant role for B cell responses in coeliac disease (with disease SNPs significantly enriched in B-cell-specific enhancers) (Kumar et al., 2015).

Patients with active coeliac disease exhibit a marked expansion of TG2-specific plasma cells within the duodenal mucosa. Further increases in extracellular IgM and IgA are evident in the lamina propria and epithelial cells in response to gluten, consistent with an active immunoglobulin response within the small intestinal mucosa (Lancaster-Smith et al., 1977). Notably TG2-specific IgM plasma cells have been described in coeliac disease, which could exert pathogenic effects via their ability to activate complement to promote inflammation. Indeed, subepithelial deposition of terminal complement complex has been observed in untreated and partially treated (but not successfully treated) patients with coeliac disease, correlating with serum levels of gluten-specific IgM and IgG (Halstensen et al., 1992).

Dermatitis herpetiformis is an itchy blistering skin disorder regarded as the cutaneous manifestation of coeliac disease (Collin et al., 2017). It is characterised by granular IgA deposits in the dermal papillae of uninvolved skin (Caja et al., 2011). Patients with dermatitis herpetiformis exhibit autoantibodies against epidermal TG3, which are gluten-dependent, and respond slowly to a gluten-free diet (Hull et al., 2008). Its pathogenesis is thought to involve active coeliac disease in the intestine resulting in the formation of IgA anti-TG3 antibody complexes in the skin.

Notably B cell depletion with rituximab has resulted in complete clinical and serological remission in a case of refractory dermatitis herpetiformis (Albers et al., 2017). Similarly, rituximab has resulted in dramatic clinical improvement in a mixed case of symptomatic coeliac disease and Sjogren's syndrome (Nikiphorou and Hall, 2014).

Thus, in an embodiment, the invention provides (i) a compound selected from clozapine, norclozapine and prodrugs thereof and pharmaceutically acceptable salts and solvates thereof for use in the treatment or prevention of a pathogenic immunoglobulin driven B cell disease in a subject and (ii) a method of treatment or prevention of a pathogenic immunoglobulin driven B cell disease in a subject by administering to said subject an effective amount of a compound selected from clozapine, norclozapine and prodrugs thereof and pharmaceutically acceptable salts and solvates thereof wherein in the case of (i) and (ii) the pathogenic immunoglobulin driven B cell disease is a disease selected from the group consisting of Pemphigus vulgaris, Pemphigus foliaceus, bullous pemphigoid, cicatricial pemphigoid, autoimmune alopecia, vitiligo, dermatitis herpetiformis, chronic autoimmune urticaria, coeliac disease, Graves' disease, Hashimoto's thyroiditis, Type 1 diabetes mellitus, autoimmune Addison's disease, autoimmune haemolytic anaemia, autoimmune thrombocytopenic purpura, cryoglobulinemia, pernicious anaemia, myasthenia gravis, multiple sclerosis, neuromyelitis optica, autoimmune epilepsy and encephalitis, autoimmune hepatitis, primary biliary cirrhosis and primary sclerosing cholangitis.

Preferably the pathogenic IgG driven B cell disease is selected from Pemphigus vulgaris, Pemphigus foliaceus and bullous pemphigoid.

Exemplary pathogenic IgA driven B cell diseases may be selected from the group consisting of the skin related diseases dermatitis herpetiformis, linear IgA disease, Pemphigus vulgaris, Pemphigus foliaceus, cicatricial pemphigoid and bullous pemphigoid. Alternatively, the disease may be the gut related disease coeliac disease. Alternatively, the disease may be the kidney related disease IgA nephropathy.

Preferably the pathogenic IgA driven B cell disease is selected from dermatitis herpetiformis and linear IgA disease.

Clozapine is associated with high levels of CNS penetration which could prove to be a valuable property in treating some of these diseases (Michel et al., 2015).

In certain diseases, more than one Ig type (such as IgG and IgA) may play a role in the pathology of the disease. For example, in dermatitis herpetiformis, coeliac disease, Pemphigus vulgaris, Pemphigus foliaceus, cicatricial pemphigoid and bullous pemphigoid, production of pathogenic IgA is thought to contribute towards the pathology as well as IgG.

In certain diseases, such as multiple sclerosis, vitiligo, Type 1 diabetes mellitus, autoimmune Addison's disease, dermatitis herpetiformis, coeliac disease, primary biliary cirrhosis, primary sclerosing cholangitis and autoimmune thrombocytopenic purpura there may also be a T cell component that contributes towards the pathology of the disease. This arises because B cells act as professional antigen-presenting cells for T cells (their importance is increased also due to their sheer numbers). B cells secrete significant amounts of cytokines that impact T cells. B-T interaction is involved in responses to T dependent protein antigens and class switching. Therefore, clozapine and norclozapine are expected to have an effect on T cells due to their effect on reducing B cell numbers.

Suitably the compound selected from clozapine, norclozapine and prodrugs thereof inhibits mature B cells, especially CSMBs and plasmablasts, particularly CSMBs. “Inhibit” means reduce the number and/or activity of said cells. Thus, suitably clozapine or norclozapine reduces the number of CSMBs and plasmablasts, particularly CSMBs.

In an embodiment the compound selected from clozapine, norclozapine and prodrugs thereof has the effect of decreasing CD19 (+) and/or CD19 (−) B-plasma cells.

The term “treatment” means the alleviation of disease or symptoms of disease. The term “prevention” means the prevention of disease or symptoms of disease. Treatment includes treatment alone or in conjunction with other therapies. Treatment embraces treatment leading to improvement of the disease or its symptoms or slowing of the rate of progression of the disease or its symptoms. Treatment includes prevention of relapse.

The term “effective amount” refers to an amount effective, at dosages and for periods of time necessary, to achieve the desired therapeutic result, in which any toxic or detrimental effects of the pharmacological agent are outweighed by the therapeutically beneficial effects. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation. Example dosages are discussed below.

As used herein, a “subject” is any mammal, including but not limited to humans, non-human primates, farm animals such as cattle, sheep, pigs, goats and horses; domestic animals such as cats, dogs, rabbits; laboratory animals such as mice, rats and guinea pigs that exhibit at least one symptom associated with a disease, have been diagnosed with a disease, or are at risk for developing a disease. The term does not denote a particular age or sex. Suitably the subject is a human subject.

It will be appreciated that for use in medicine the salts of clozapine and norclozapine should be pharmaceutically acceptable. Suitable pharmaceutically acceptable salts will be apparent to those skilled in the art. Pharmaceutically acceptable salts include those described by Berge, Bighley and Monkhouse J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of clozapine and are included within the scope of this invention.

A compound selected from clozapine, norclozapine and prodrugs thereof and pharmaceutically acceptable salts and solvates thereof may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water).

A “prodrug”, such as an N-acylated derivative (amide) (e.g. an N-acylated derivative of norclozapine) is a compound which upon administration to the recipient is capable of providing (directly or indirectly) clozapine or an active metabolite or residue thereof. Other such examples of suitable prodrugs include alkylated derivatives of norclozapine other than clozapine itself.

Isotopically-labelled compounds which are identical to clozapine or norclozapine but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”) are also contemplated for the uses and method of the invention. Examples of isotopes that can be incorporated into clozapine or norclozapine include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I which may be naturally occurring or non-naturally occurring isotopes.

Clozapine or norclozapine and pharmaceutically acceptable salts of clozapine or norclozapine that contain the aforementioned isotopes and/or other isotopes of other atoms are contemplated for use for the uses and method of the present invention. Isotopically labelled clozapine or norclozapine, for example clozapine or norclozapine into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Since clozapine or norclozapine are intended for use in pharmaceutical compositions it will readily be understood that it is preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

In general, clozapine or norclozapine may be made according to the organic synthesis techniques known to those skilled in this field (as described in, for example, U.S. Pat. No. 3,539,573.

A compound selected from clozapine, norclozapine and prodrugs thereof and pharmaceutically acceptable salts and solvates thereof for use in therapy is usually administered as a pharmaceutical composition. Also provided is a pharmaceutical composition comprising clozapine or norclozapine, or a pharmaceutically acceptable salt and/or solvate and/or prodrug thereof and a pharmaceutically acceptable diluent or carrier. Said composition is provided for use in the treatment or prevention of a pathogenic immunoglobulin driven B cell disease in a subject wherein said compound causes mature B cells to be inhibited in said subject.

A compound selected from clozapine, norclozapine and prodrugs thereof and pharmaceutically acceptable salts and solvates thereof may be administered by any convenient method, e.g. by oral, parenteral, buccal, sublingual, nasal, rectal or transdermal administration, and the pharmaceutical compositions adapted accordingly. Other possible routes of administration include intratympanic and intracochlear. Suitably, a compound selected from clozapine, norclozapine and prodrugs thereof and pharmaceutically acceptable salts and solvates thereof are administered orally.

A compound selected from clozapine, norclozapine and prodrugs thereof and pharmaceutically acceptable salts and solvates thereof which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal or pulmonary administration may conveniently be formulated as aerosols, sprays, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device.

Alternatively the sealed container may be a disposable dispensing device such as a single dose nasal or pulmonary inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatine and glycerine.

Compositions for rectal administration are conveniently in the form of suppositories containing a conventional suppository base such as cocoa butter.

Compositions suitable for topical administration to the skin include ointments, gels and patches.

In one embodiment the composition is in unit dose form such as a tablet, capsule or ampoule.

Compositions may be prepared with an immediate release profile upon administration (i.e. upon ingestion in the case of an oral composition) or with a sustained or delayed release profile upon administration.

For example, a composition intended to provide constant release of clozapine over 24 hours is described in WO2006/059194 the contents of which are herein incorporated in their entirety.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material (i.e. clozapine or norclozapine), depending on the method of administration. The composition may contain from 50 mg to 1000 mg, for example from 100 mg to 400 mg of the carrier, depending on the method of administration. The dose of clozapine or norclozapine used in the treatment or prevention of the aforementioned diseases will vary in the usual way with the seriousness of the diseases, the weight of the sufferer, and other similar factors. However, as a general guide suitable unit doses of clozapine as free base may be 0.05 to 1000 mg, more suitably 1.0 to 500 mg, and such unit doses may be administered more than once a day, for example two or three a day. Such therapy may extend for a number of weeks or months.

A compound selected from clozapine, norclozapine and prodrugs thereof and pharmaceutically acceptable salts and solvates thereof may be administered in combination with another therapeutic agent for the treatment of pathogenic immunoglobulin driven B cell diseases (e.g. IgG or IgA driven B cell disease), such as those that inhibit B cells or B cell-T-cell interactions. Other therapeutic agents include for example: anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab). Rituximab may be mentioned in particular.

Other therapies that may be used in combination with the invention include non-pharmacological therapies such as intravenous immunoglobulin therapy (IVIg), subcutaneous immunoglobulin therapy (SCIg) eg facilitated subcutaneous immunoglobulin therapy, plasmapheresis and immunoabsorption.

Thus the invention provides a compound selected from clozapine, norclozapine and prodrugs thereof and pharmaceutically acceptable salts and solvates thereof for use in the treatment or prevention of a pathogenic immunoglobulin driven B cell disease in combination with a second or further therapeutic agent for the treatment or prevention of a pathogenic immunoglobulin driven B cell disease (e.g. IgG or IgA driven B cell disease) e.g. a substance selected from the group consisting of anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or and azathioprine), general anti-inflammatories (such as hydroxychloroquine and NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab). Rituximab may be mentioned in particular.

When a compound selected from clozapine, norclozapine and prodrugs thereof and pharmaceutically acceptable salts and solvates thereof is used in combination with other therapeutic agents, the compounds may be administered separately, sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations. The individual components of combinations may also be administered separately, through the same or different routes. For example, a compound selected from clozapine, norclozapine and prodrugs thereof and pharmaceutically acceptable salts and solvates thereof and the other therapeutic agent may both be administered orally. Alternatively, a compound selected from clozapine, norclozapine and prodrugs thereof and pharmaceutically acceptable salts and solvates thereof may be administered orally and the other therapeutic agent via may be administered intravenously or subcutaneously.

Typically, a compound selected from clozapine, norclozapine and prodrugs thereof and pharmaceutically acceptable salts and solvates thereof is administered to a human.

### EXAMPLES

### Example 1

### First Observational Study on Human Patients on Anti-Psychotic Therapy

To assess a possible association between antibody deficiency and clozapine use the inventors undertook a cross-sectional case control study to compare the immunoglobulin levels and specific antibody levels (against Haemophilus B (Hib), Tetanus and Pneumococcus) in patients taking either clozapine or alternative antipsychotics.

### Method

Adults (>18 yrs) receiving either clozapine or non-clozapine antipsychotics were recruited during routine clinic visits to ten Community Mental Health Trust (CMHT) outpatient clinics in Cardiff & Vale and Cwm Taf Health Boards by specialist research officers between November 2013 and December 2016 (Table 1). Following consent, participants completed a short lifestyle, drug history and infection questionnaire followed by blood sampling. Where required, drug histories were confirmed with the patient's General Practice records. Formal psychiatric diagnoses and antipsychotic medication use were confirmed using the medical notes, in line with other studies. Patients' admission rates were confirmed by electronic review for the 12-month period prior to recruitment. Patients with known possible causes of hypogammaglobulinemia including prior chemotherapy, carbamazepine, phenytoin, antimalarial agents, captopril, high-dose glucocorticoids, hematological malignancy and 22q11 deletion syndrome were excluded.

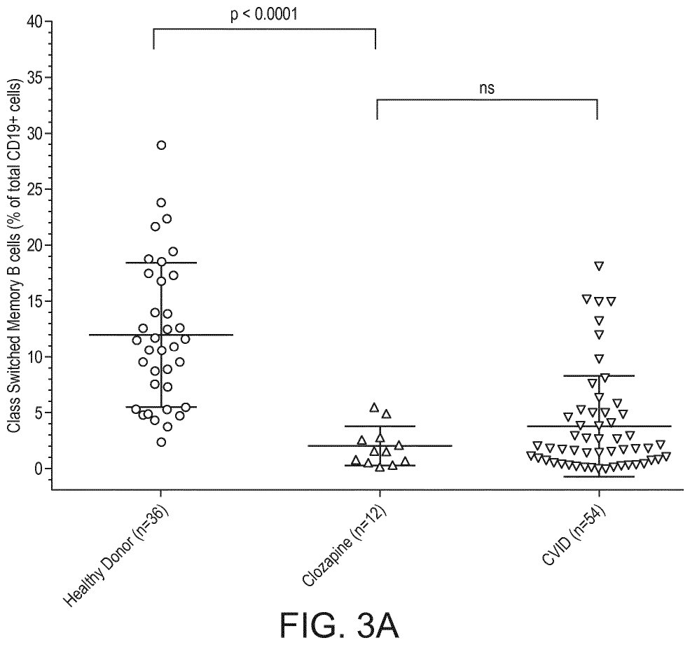

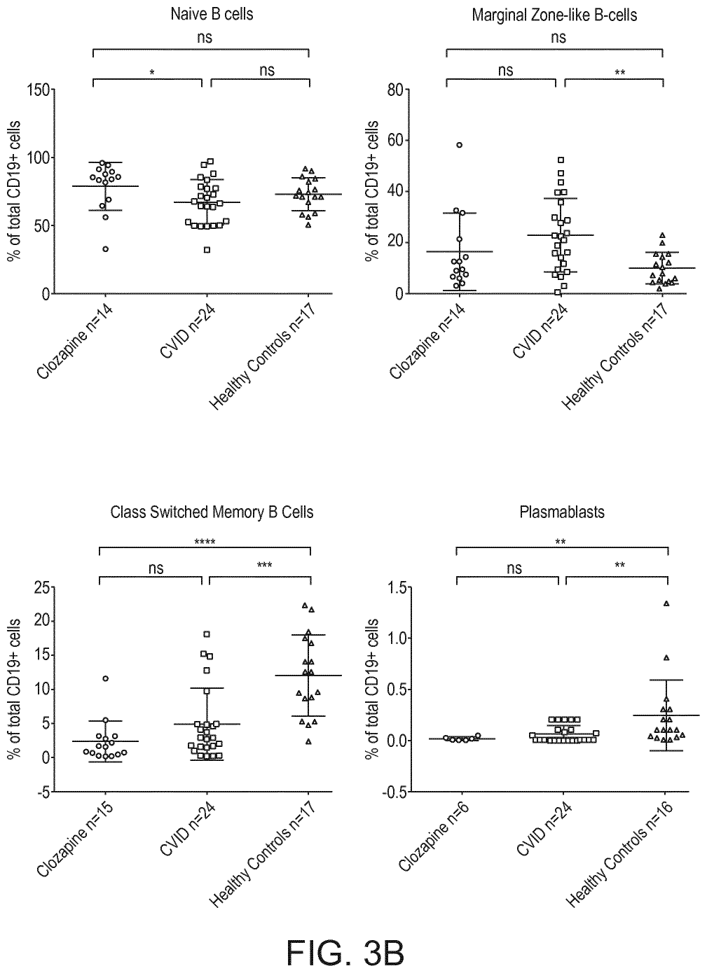

Clinical and immunological data from 13 patients taking clozapine, 11 of whom had been referred independently of the study for assessment in Immunology clinic, are presented in Table 3. Laboratory data on these, healthy controls and patients with common variable immunodeficiency (CVID) are shown in FIG. 3. The 11 independently referred patients were excluded from the overall study analysis.

Immunoglobulin levels (IgG, IgA and IgM) were assayed by nephelometry (Siemens BN2 Nephelometer; Siemens), serum electrophoresis (Sebia Capillarys 2; Sebia, Norcross, Ga., USA) and, where appropriate, serum immunofixation (Sebia Hydrasys; Sebia, Norcross, Ga., USA). Specific antibody titres against Haemophilus influenzae, Tetanus and Pneumococcal capsular polysaccharide were determined by ELISA (The Binding Site, Birmingham, UK). Lymphocyte subsets, naïve T cells and EUROclass B cell phenotyping were enumerated using a Beckman Coulter FC500 (Beckman Coulter, California, USA) flow cytometer. All testing was performed in the United Kingdom Accreditation Service (UKAS) accredited Immunology Laboratory at the University Hospital of Wales. Laboratory adult reference ranges for immunoglobulin levels used were, IgG 6-16 g/L, IgA 0.8-4 g/L, IgM 0.5-2 g/L.

Statistical analysis of the laboratory and clinical data was performed using Microsoft Excel and Graphpad Prism version 6.07 (Graphpad, San Diego, Calif., USA). Independent samples t-test were performed unless D'Agoustino & Pearson testing showed significant deviation from the Gaussian distribution, in which case the non-parametric Mann-Whitney test was used. All tests were two-tailed, using a significance level of p<0.05.

### Results

### Study Participants

A total of 291 patients taking clozapine and 280 clozapine-naïve patients were approached and 123 clozapine and 111 clozapine-naïve patients consented to the study (Table 1). Recruitment was stopped as per protocol when the target of 100 patients in each group had been achieved. There were small differences in gender with more males in the clozapine-treated group (53% versus 50%) and a lower mean age in the clozapine group (45 versus 50 years). These differences are unlikely to be relevant as there are no gender differences in the adult reference range for serum immunoglobulins and there is a male predominance in schizophrenia. Levels of smoking, diabetes, COPD/asthma, and alcohol intake were similar between the groups. More patients were admitted to hospital with infection in the clozapine group (0.12 vs 0.06 per patient year) and more took >5 courses of antibiotics per year compared with controls (5.3% vs 2%). The possible impact of a diagnosis of schizophrenia, medications and smoking as risk factors for antibody deficiency were assessed in a subgroup analysis (Table 2).

TABLE 1
Clozapine-treated and clozapine-naïve patient characteristics

| | Clozapine-Treated | | Clozapine-Naive | |
|---|---|---|---|---|
| Total screened | 291 | | 280 | |
| Declined, lacked capacity, or | 168 | | 169 | |
| unable to obtain blood sample | | | | |
| Initial Screening Sex (M:F) | 123 | (81:42) | 111 | (56:55) |
| Mean age, years (Range) | 45.3 | (22.0-78.0) | 50.3 | (21.6-78.0) |
| Post-exclusion (% total | 94 | (32%) | 98 | (35%) |
| screened) | | | | |
| Sex (M:F) | 64:30 | | 54:44 | |
| Mean age, years (Range) | 44.4 | (22.0-78.0) | 50.4 | (21.6-78.0) |
| Primary Psychiatric Diagnosis | | | | |
| Schizophrenia | 87 | | 58 | |
| Schizoaffective | 1 | | 5 | |
| Bipolar | 0 | | 11 | |
| Psychosis | 0 | | 15 | |
| Depression | 0 | | 3 | |
| Personality Disorder | 2 | | 2 | |
| Anxiety disorder | 0 | | 2 | |
| Electronic record incomplete | 4 | | 2 | |
| Dual antipsychotic treatment | 30.9% | | 11.2% | |
| Duration antipsychotic use | 8.0 | (0.1-20) | 7.0 | (0.1-44) |
| (median, range), years | | | | |
| Current smoking (%) | 60.6% | | 56.1% | |
| Diabetes (%) | 20.2% | | 17.3% | |
| COPD/Asthma (%) | 13.8% | | 16.3% | |
| Alcohol intake mean (units/ | 5.3 | (0-60) | 6.0 | (0-68) |
| week), range | | | | |
| Antibiotic courses per year | | | | |
| Nil courses | 61.7% | | 63.3% | |
| 1-5 courses | 33.0% | | 34.7% | |
| >5 courses | 5.3% | | 2.0% | |
| Admission frequency in 12- | | | | |
| month period | | | | |
| All cause | 21 | (14 patients) | 14 | (13 patients) |
| Infection-related | 15 | (10 patients) | 7 | (6 patients) |

### Effects of Clozapine on Antibody Levels

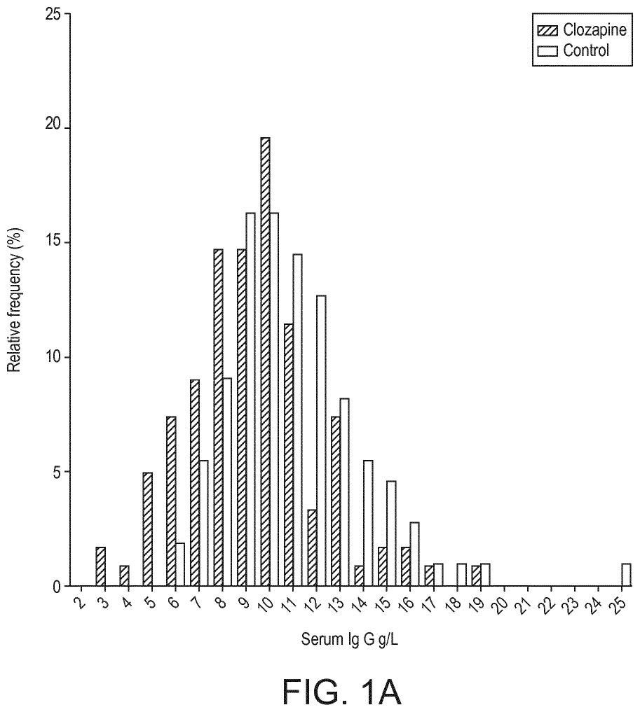

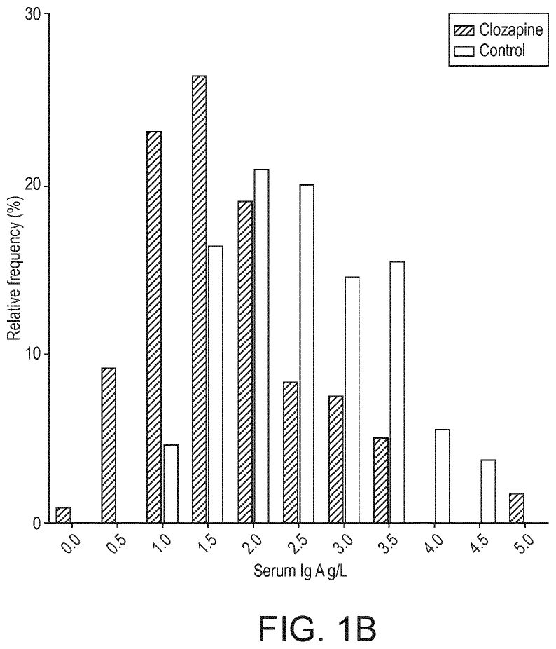

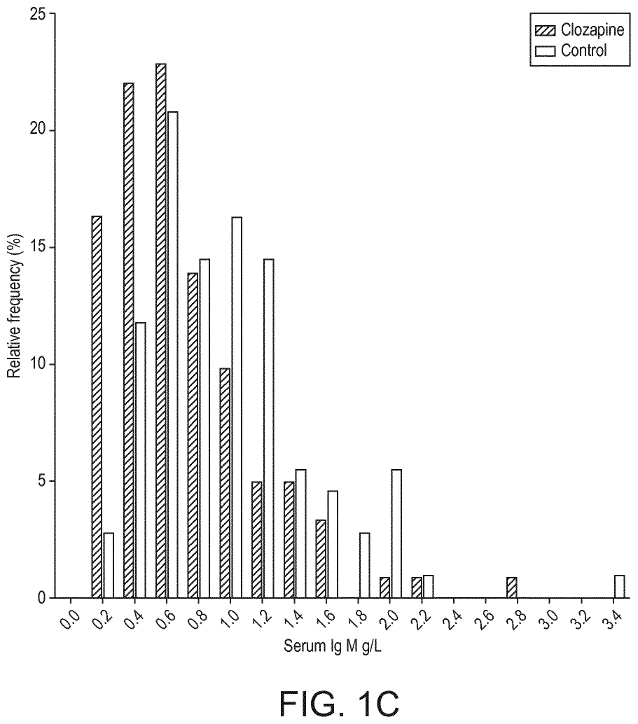

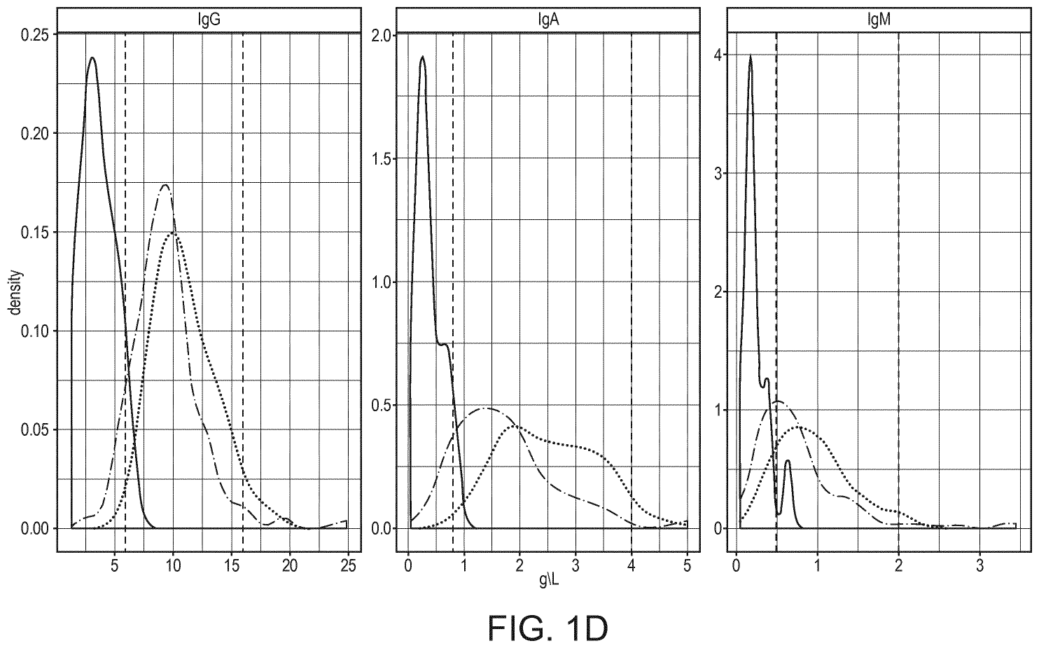

FIG. 1 A-C shows significantly reduced concentrations of all three immunoglobulin classes (IgG, IgA and IgM) in patients receiving clozapine, with a shift towards lower immunoglobulin levels in the distribution as a whole for each of IgG, IgA and IgM compared to the clozapine-naïve control group. The percentages of the 123 patients having immunoglobulin levels below the reference range were IgG 9.8% (p<0.0001), IgA 13.0% (p<0.0001) and IgM 38.2% (p<0.0001) compared with the 111 clozapine-naïve IgG 1.8%, IgA 0.0% and IgM 14.4%. Large percentages of both clozapine-treated and clozapine-naïve patients had specific antibody levels below the protective levels for HiB (51% and 56% less than 1 mcg/ml, (Orange et al., 2012)), Pneumococcus (54% and 56% less than 50 mg/L, (Chua et al., 2011)) and Tetanus (12% and 14% less than 0.11 U/ml). The Pneumococcal IgA (31 U/ml vs 58.4 U/ml p<0.001) and IgM (58.5 U/ml vs 85.0 U/ml p<0.001) levels are significantly lower in clozapine-treated versus clozapine-naïve patients.

Subgroup analysis (Table 2) was undertaken to determine if the reductions in immunoglobulins were potentially explained by confounding factors including any other drugs, a diagnosis of schizophrenia and smoking. The assessment of the effect of excluding other secondary causes of antibody deficiency (plus small numbers where additional diagnoses were uncovered—Table 1) is shown in Column B. The number of patients excluded on the basis of taking anti-epileptic medications was higher in the clozapine-treated group and is likely to reflect the use of these agents for their mood stabilizing properties rather than as treatment for epilepsy.

TABLE 2
Immunoglobulin levels and specific antibody levels in sub-groups A-D

| | A | | B | | C | | D | |
|---|---|---|---|---|---|---|---|---|
| Medication: | Clozapine | Control | Clozapine | Control | Clozapine | Control | Clozapine | Control |
| Diagnosis: | All | All | All | All | Schizophrenia | | All | All |
| | | | | | diagnoses only | | | |
| Smoking: | All | All | All | All | All | All | Smokers only | |
| Possible | No | No | Yes | | Yes | | Yes | |
| secondary | | | | | | | | |
| causes excluded | | | | | | | | |
| Sample size: | 123 | 111 | 94 | 98 | 87 | 58 | 57 | 55 |
| Serum IgG | 95% CI: 0.89- | | 95% CI: 0.98 | | 95% CI: 0.92 | | Non-Gaussian | |
| (Reference range | 2.32 **** | | to 2.59 **** | | to 2.77 *** | | distribution ^{†} | |
| 6-16 g/L) | | | | | | | | |
| <3 | 0.8% | 0.0% | 1.1% | 0.0% | 1.2% | 0.0% | 1.8% | 0.0% |
| <4 | 1.6% | 0.0% | 1.1% | 0.0% | 1.2% | 0.0% | 1.8% | 0.0% |
| <5 | 3.3% | 0.0% | 2.1% | 0.0% | 2.3% | 0.0% | 1.8% | 0.0% |
| <6 | 9.8% | 1.8% | 8.4% | 1.0% | 9.2% | 1.7% | 8.8% | 1.8% |
| Serum IgA | 95% CI: 0.55 | | 95% CI: 0.55 | | 95% CI: 0.59 | | 95% CI: 0.41 | |
| (Reference range | to 1.01 **** | | to 1.05 **** | | to 1.19 **** | | to 1.04 **** | |
| 0.8-4.0 g/L) | | | | | | | | |
| <0.5 | 1.6% | 0.0% | 2.1% | 0.0% | 2.3% | 0.0% | 3.5% | 0.0% |
| <0.6 | 2.4% | 0.0% | 2.1% | 0.0% | 3.5% | 00% | 3.5% | 0.0% |
| <0.7 | 6.5% | 0.0% | 6.4% | 0.0% | 6.9% | 0.0% | 3.5% | 0.0% |
| <0.8 | 13.0% | 0.0% | 13.8% | 0.0% | 14.9% | 0.0% | 10.5% | 0.0% |
| Serum IgM | Non-Gaussian | | 95% CI: 0.10 | | 95% CI: 0.06 | | 95% CI: 0.02 | |
| (Reference range | distribution ^{††††} | | to 0.38 *** | | to 0.38 ** | | to 0.39 * | |
| 0.5-1.9 g/L) | | | | | | | | |
| <0.2 | 8.1% | 0.0% | 5.3% | 0.0% | 5.8% | 0.0% | 1.78% | 0.0% |
| <0.3 | 16.3% | 2.7% | 12.8% | 3.1% | 12.6% | 5.2% | 12.3% | 1.8% |
| <0.4 | 29.3% | 8.1% | 26.6% | 8.2% | 27.6% | 6.9% | 26.3% | 9.1% |
| <0.5 | 38.2% | 14.4% | 34.0% | 15.3% | 35.6% | 13.8% | 33.3% | 18.2% |
| IgG- | 95% CI: −8.25 | | 95% CI: −11.21 | | 95% CI: −20.50 | | 95% CI: −23.64 | |
| Pneumococcus | to 21.92 (ns) | | to 22.63 (ns) | | to 17.54 (ns) | | to 21.70 (ns) | |
| (mg/L) | | | | | | | | |
| <35 | 39.0% | 43.2% | 38.3% | 40.8% | 37.9% | 43.1% | 45.6% | 43.6% |
| <50 | 53.7% | 55.9% | 52.1% | 54.1% | 50.6% | 60.3% | 54.4% | 63.6% |
| IgG- Tetanus | Non-Gaussian | | Non-Gaussian | | Non-Gaussian | | Non-Gaussian | |
| (IU/ml) | distribution (ns) | | distribution (ns) | | distribution (ns) | | distribution (ns) | |
| <0.1 | 12.2% | 13.5% | 10.6% | 13.3% | 11.5% | 13.8% | 12.3% | 14.6% |
| IgG- | Non-Gaussian | | Non-Gaussian | | Non-Gaussian | | Non-Gaussian | |
| Haemophilus B | distribution (ns) | | distribution (ns) | | distribution (ns) | | distribution (ns) | |
| (mcg/ml) | | | | | | | | |
| <1.0 | 51.2% | 55.9% | 51.1% | 54.1% | 49.4% | 53.5% | 50.9% | 60.0% |
| Sample size: | 118 | 85 | 89 | 77 | 84 | 45 | 54 | 45 |
| IgA- | 31 ± | 58.4 ± | 30.8 ± | 58.8 ± | 31.6 ± | 49.9 ± | 30.7 ± | 61.3 ± |
| Pneumococcus | 3.97 *** | 6.7 | 4.7 ^{††††} | 7.0 | 4.9 ^{†††} | 7.6 | 5.7 ^{††††} | 9.5 |
| (U/) | | | | | | | | |
| IgM- | 58.5 + | 85 ± | 59.8 ± | 85.8 ± | 60.4 ± | 78.6 ± | 61.6 ± | 91.7 ± |
| Pneumococcus | 4.2 *** | 6.9 | 4.9 ** | 7.4 | 5.1 * | 7.1 | 7.0 ^{††} | 10.3 |
| (U/L) | | | | | | | | |

Data shown as mean ± 1 SEM unless otherwise stated.
* Independent T test (normally distributed) or
^{†} Mann-Whitney (non-normally distributed)
Levels of significance:
^{†}/* p < 0.05,
**/^{††} p < 0.005,
***/^{†††} p < 0.0005,
****/^{††††} p < 0.0001

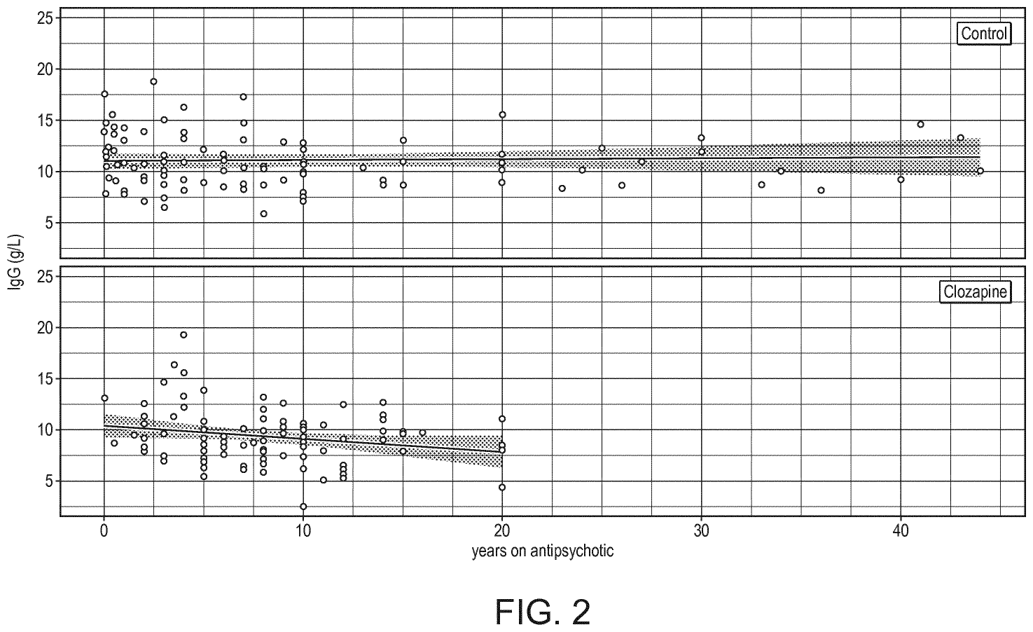

The association of clozapine with reduced IgG, IgA, IgM and Pneumococcal IgA and IgM remained statistically significant in all subgroups with 95% confidence intervals including when psychiatric diagnoses were restricted to schizophrenia only (Column C), and when non-smokers were excluded (Column D). When secondary causes of antibody deficiency were excluded (Column B) the odds ratios (with 95% confidence interval) for reduced immunoglobulins were IgG 9.02 (1.11-73.7), IgA: 32.6 (1.91-558) and IgM: 2.86 (1.42-5.73). In addition, a longer duration of clozapine therapy is associated with lower serum IgG levels (p 0.014) shown in FIG. 2. This is not observed in clozapine-naïve patients treated with alternative antipsychotic drugs, despite a longer treatment duration than the clozapine therapy group.

### Immunological Assessment of Referred Patients Taking Clozapine

Thirteen patients on clozapine were independently referred for assessment of antibody deficiency to Immunology clinic. Two had previously been recruited to the study and the eleven others are not included in the study to avoid bias. Five of the thirteen patients had been identified through the all Wales calculated globulin screening program. It was thus possible to undertake a more detailed immunological assessment in this group of thirteen ‘real life’ patients to provide additional background information (Table 3).

TABLE 3
Immunological characteristics of the 13 referred clozapine patients

| Referral | | | Relevant | Clozapine | CSMB | | | Follow-up/ |
|---|---|---|---|---|---|---|---|---|
| Reason | Age | Smoking | Medication | duration | (6.5-29.1%) | | Intervention | months |
| Recurrent | 47 | 20 | Clozapine | >4 | IgG < 1.34 | 0.3% | Prophylactic antibiotics | 120 |
| respiratory | | pack | 250 mg | | IgA < 0.22 | | Failure to respond to | |
| tract infection | | years | Sodium | | IgM < 0.17 | | haemophilus and | |
| (12 per year). | | | Valproate 1 g | | | | pneumococcal vaccination. | |
| | | | Risperidone | | | | Commenced SCIg 9.6 g weekly | |
| | | | | | | | in nursing home. | |
| | | | | | | | Recently discontinued | |
| | | | | | | | clozapine due to | |
| | | | | | | | neutropenia. | |
| Low | 46 | 42 | Clozapine | 15 | IgG 5.24 | 2.77% | Prompt antibiotic therapy | 69 |
| calculated | | pack | 575 mg | | IgA 0.49 | | Durable pneumococcal | |
| globulin | | years | Senna, | | IgM 0.41 | | vaccine response | |
| Included in | | | fibrogel, | | | | Continues clozapine | |
| study | | | cyclizine | | | | | |
| Low | 51 | 34 | Clozapine | 5 | IgG 2.68 | 5.50% | Prophylactic antibiotics | 48 |
| calculated | | pack | 200 mg | | IgA 0.38 | | Failure to responds to | |
| globulin. | | years | Amisulpride | | IgM < 0.17 | | haemophilus and | |
| | | | | | | | pneumococcal vaccination. | |
| | | | | | | | Continues clozapine, | |
| | | | | | | | Considering immunoglobulin | |
| | | | | | | | replacement | |
| Persistent | 63 | 60 | Clozapine | 7.5 | IgG 2.98 | 0.5% | Prophylactic antibiotics | 42 |
| cough for | | pack | 400 mg | | IgA < 0.22 | | Non-durable pneumococcal | |
| over a year | | years | Olanzapine | | IgM 0.23 | | vaccine response | |
| and remains | | | Trihexyphenidyl | | | | Commenced IVIg 40 g 3 | |
| productive of | | | | | | | weekly | |
| green sputum | | | | | | | Clozapine stopped with | |
| despite | | | | | | | resultant psychotic episode. | |
| several | | | | | | | Clozapine restarted with | |
| courses of | | | | | | | GCSF cover | |
| antibiotics. | | | | | | | Continues on SCIg and | |
| | | | | | | | clozapine. | |
| Recurrent | 49 | 55 | Clozapine | 7 | IgG 1.2 | 0.14% | Prophylactic antibiotics | 32 |
| respiratory | | pack | 300 mg | | IgA | | Failure to respond to | |
| infections | | years | Sodium | | undetectable | | pneumococcal vaccination. | |
| Low | | | Valproate, | | IgM 0.07 | | IVIg 40 g 3 weekly | |
| calculated | | | Pirenzapine, | | | | Continues clozapine | |
| globulins | | | aripiprazole | | | | | |
| Recurrent | 63 | 20 | Clozapine | 10 years | IgG 3.3 | 1.58% | Prophylactic azithromycin: 4 | 24 |
| chest | | pack | 250 mg - | Stopped | IgA 0.26 | | chest infections in 3 months | |
| infections | | years, | stopped | 24 | IgM 0.41 | | Failure to respond to | |
| Low | | stopped 30 | Lithium | months | | | pneumococcal vaccination | |
| calculated | | years | 400 mg | ago | | | Clozapine stopped- red flags | |
| globulin | | ago | Levothyroxine | | | | with neutropenia | |
| | | | Calchichew | | | | IgG rose to 5.95 from 3.3 g/L, | |
| | | | Citalopram | | | | IgA 0.29, IgM 0.49 after 24 | |
| | | | | | | | months | |
| | | | | | | | CSMB rose to 2.77% | |
| 7 courses of | 59 | 47 | Clozapine | 10 | IgG 2.38 | 2.54% | Prophylactic antibiotics | 15 |
| antibiotics for | | pack | 450 mg | | IgA < 0.22 | | Failure to respond to | |
| chest | | years | Omeprazole, | | IgM < 0.17 | | pneumococcal vaccination. | |
| infections | | | pirenzapine, | | | | Commenced IVIg 30 g 3- | |
| past 12 | | | venlafaxine, | | | | weekly | |
| months, 9 GP | | | metformin, | | | | Continues clozapine | |
| visits | | | saxagliptin, | | | | | |
| No clozapine | | | atorvastatin | | | | | |
| red-flags | | | | | | | | |
| Included in | | | | | | | | |
| study | | | | | | | | |
| Recurrent | 46 | 74 | Clozapine | 21 | IgG 4.24 | 0.84% | Prophylactic antibiotics | 12 |
| respiratory | | pack | 450 mg | | IgA < 0.22 | | Failure to respond to | |
| infections | | years | Sertaline, | | IgM < 0.17 | | pneumococcal vaccination. | |
| | | | montelukast, | | | | Commenced SCIg | |
| | | | simvastatin, | | | | Continues clozapine | |
| | | | seretide, | | | | | |
| | | | salbulatamol, | | | | | |
| | | | temazepam | | | | | |
| Recurrent | 50 | 60 | Clozapine | >7 | IgG 6.65 | 4.95% | Prophylactic antibiotics | 12 |
| respiratory | | pack | 700 mg | | IgA < 0.22 | | Failure to responds to | |
| tract | | years | Amisulpride, | | IgM < 0.17 | | haemophilus and | |
| infections | | | cholecalciferol, | | | | pneumococcal vaccination. | |
| | | | cod liver oil | | | | Continues clozapine | |
| Low | 51 | 12 | Clozapine | 11 | IgG 5.61 | 2.10% | Prompt antibiotic therapy | 6 |
| calculated | | pack | 575 mg | | IgA 0.81 | | Failure to respond to | |
| globulin | | years | Fibrogel, | | IgM 0.18 | | pneumococcal vaccination. | |
| | | | lactulose, cod | | | | Continues clozapine | |
| | | | liver oil, | | | | | |
| | | | citalopram | | | | | |
| Recurrent | 61 | 15/day | Clozapine | >4 | IgG 4.79 | 1.49% | Prompt antibiotics | 6 |
| skin infections | | | 325 mg | | IgA 0.63 | | Assessment of vaccine | |
| | | | Sodium | | IgM < 0.17 | | responses ongoing | |
| | | | valproate, | | | | Continues clozapine | |
| | | | metformin, | | | | | |
| | | | exenatide, | | | | | |
| | | | ciitalopram, | | | | | |
| | | | Fultium D3, | | | | | |
| | | | Omeprazole, | | | | | |
| Calculated | 36 | 35 | Clozapine - | Stopped | IgG 4.8 | N/A | Declined further blood tests | 5 |
| globulin | | pack | stopped 2 | | IgA 0.54 | | | |
| | | years | years prior to | | IgM 0.3 | | | |
| | | | referral | | | | | |
| | | | Procyclidine, | | | | | |
| | | | folic acid, | | | | | |
| | | | diazepam, | | | | | |
| | | | paracetamol | | | | | |
| Recurrent | 57 | 20-40 | Clozapine | >4 | IgG < 1.34 | 0.3-0.7% | Prophylactic antibiotics | 42 |
| respiratory | | pack | 750 mg | | IgA < 0.22 | | Failure to respond to | |
| tract | | years | Amisulpride | | IgM < 0.17 | | pneumococcal vaccination | |
| infections. | | | | | | | IVIg 40 g every 3 weekly | |
| Clozapine- | | | | | | | Stopped clozapine during | |
| induced | | | | | | | chemotherapy | |
| sialorrhoea. | | | | | | | | |

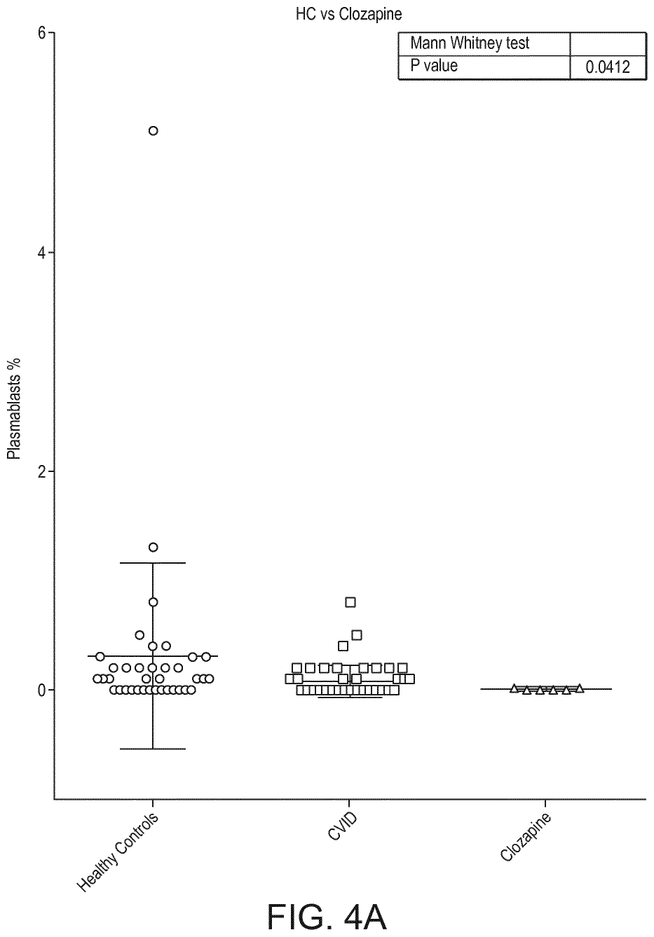

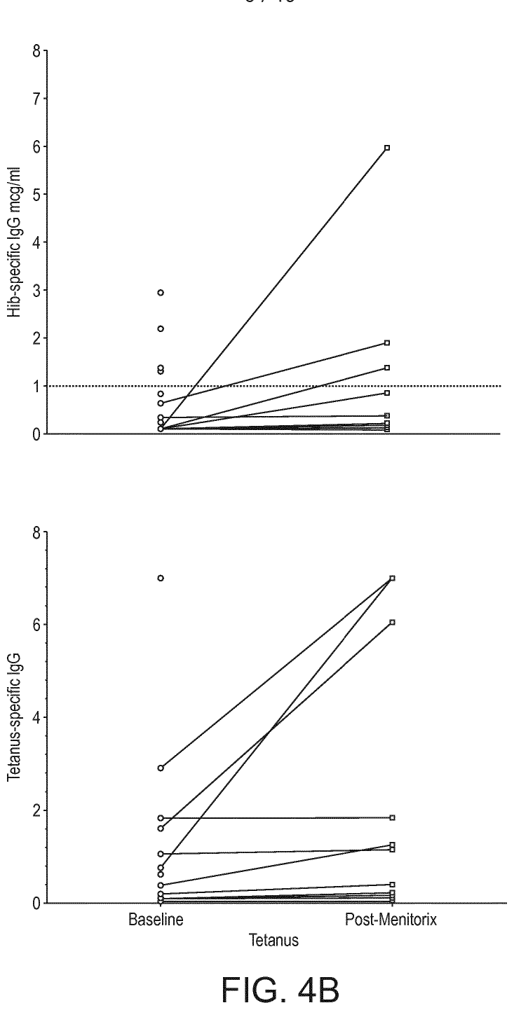

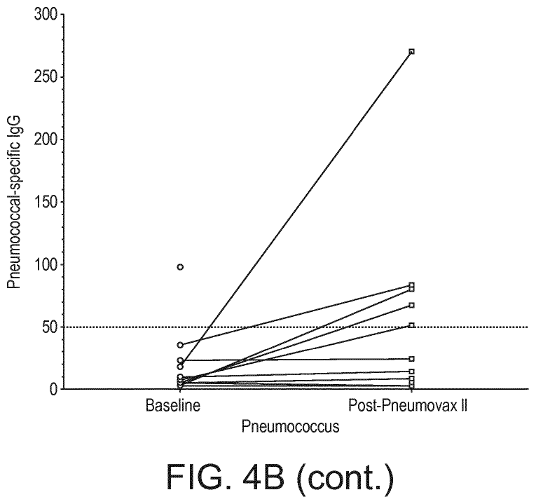

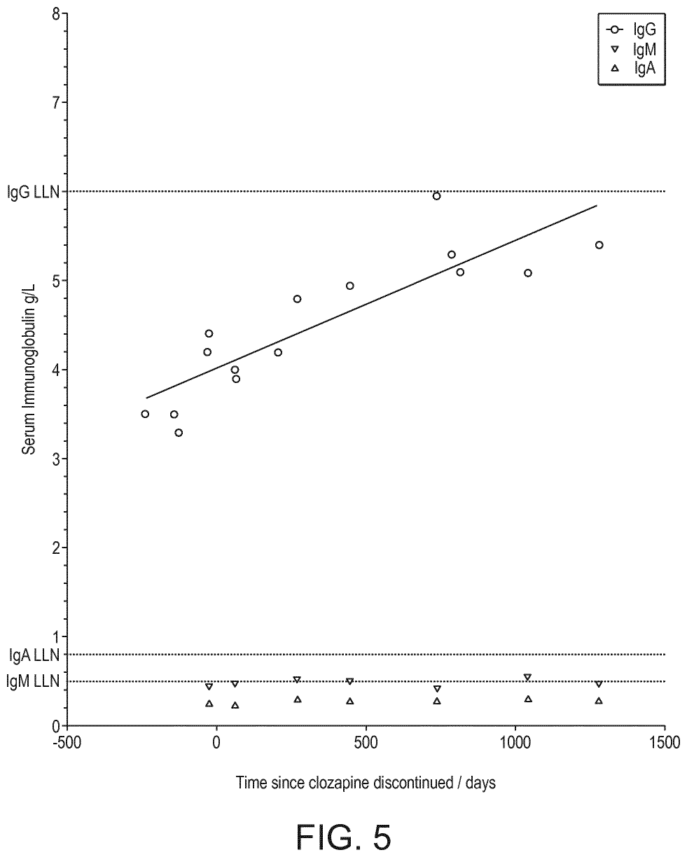

Certain additional analysis shown in FIGS. 1D, 3B, 4B and 5 was done on a slightly different set of referred clozapine patients comprising the 13 referred to in Table 3, plus 4 additionally recruited patients. In respect of FIG. 1D, 4 of the 17 patients were removed for various reasons therefore the number of patients for which data is presented is 13. In respect of FIG. 3B, the number of patients for which data is presented is shown in the Figure. In respect of FIG. 4B, the number of patients for which data is presented is stated below. In respect of FIG. 5, the number of patients for which data is presented is 15.

Immunoglobulins were reduced in all patients (mean IgG 3.6 g/L, IgA 0.34 g/L and IgM 0.21 g/L). There was no severe overall lymphopenia or B cell lymphopenia, however, all patients had a major reduction in the percentage of CSMB (mean 1.87%, reference range 6.5-29.1%). A substantial reduction of CSMB is characteristic of patients with common variable immunodeficiency (CVID), the commonest severe primary immunodeficiency in adults. The percentages of CSMB in these clozapine-treated and CVID patients compared to healthy controls are shown in FIG. 3A (p<0.0001). The plasmablast levels for 6 of the clozapine patients compared to CVID patients and healthy controls are shown in FIG. 4A (p=0.04) and in FIG. 3B with age matched CVID and healthy controls. A reduction of plasmablasts is also characteristic of patients with common variable immunodeficiency (CVID) and this was also observed in clozapine treated patients. Responses to vaccination were impaired in 10/11 patients assessed and management included emergency backup antibiotics for 2/13 patients, prophylactic antibiotics in 9/13 and 6/13 patients were treated with immunoglobulin replacement therapy (IGRT). No patients discontinued clozapine because of antibody deficiency. The inflammatory or granulomatous complications which occur in a subset of CVID patients were not observed.

Vaccine specific-IgG responses are routinely evaluated as part of clinical assessment and summarised in FIG. 4B. At initial assessment, levels below putative protective threshold were common with IgG to Haemophilus influenza B (HiB)<1 mcg/ml in 12/16 patients (75%); Pneumococcus-IgG <50 mg/L in 15/16 patients (94%); and Tetanus-IgG <0.1 IU/mL in 6/16 patients (38%) individuals tested. Post-Menitorix (HiB/MenC) vaccination serology was assessed after 4 weeks, with 5/12 (42%) individuals failing to mount a Haemophilus-IgG response ≥1.mcg/ml, and 1/12 failing to exceed the ≥0.1 IU/mL post-vaccination Tetanus-IgG level defined by the World Health Organisation. Following Pneumovax II, 8/11 (73%) individuals failed to develop an IgG response above a threshold of ≥50 mg/L.

FIG. 5 shows a gradual recovery in terms of the serum IgG level from 3.5 g/L to 5.95 g/L over 3 years but without clear improvement in IgA or IgM following cessation of clozapine.

One patient subsequently discontinued clozapine because of neutropenia which normalized on clozapine cessation. Over the following 24 months the serum IgG level gradually increased from 3.3 g/L to 4.8 g/L and then 5.95 g/L while IgA and IgM remained low. The increase in IgG was accompanied by a concomitant increase in class switched memory B cells from 1.58-2.77%, suggesting a gradual recovery on withdrawal of clozapine.

FIG. 1D shows a density plot showing distribution of serum immunoglobulin levels in patients receiving clozapine referred for Immunology assessment. Serum immunoglobulin distributions for clozapine-treated (n=94) and clozapine-naive (n=98) are also shown for comparison-adapted from (Ponsford et al., 2018b). Dotted lines represent the 5^{th} and 95^{th} percentiles for healthy adults. A leftward shift (reduction) in the distribution curves of total immunoglobulin is observed in patients on clozapine for each of IgG, IgA and IgM compared to clozapine naive patients; this finding was particularly marked for the additionally recruited clozapine referred patients.

### Summary of Results

Clozapine treatment in patients led to a significant reduction of all immunoglobulin types. Percentages of patients below the immunoglobulin reference ranges were higher in clozapine treated (n=123) as compared with clozapine naive patients (n=111) (IgG <6 g/L: 9.8% vs 1.8%; IgA <0.8 g/L: 13.1% vs 0.0%; IgM <0.5 g/L: 38.2% vs 14.2%) (p<0.0001) (see FIG. 1 A-C).

Extending the duration of clozapine treatment was associated with progressively reduced IgG levels in patients treated with clozapine but not in clozapine naive patients who were on other antipsychotic medication (see FIG. 2).

Notably the effect of clozapine on IgG levels was seen to be reversible, albeit slowly (years), consistent with an impact of clozapine on long-lived IgG+ plasma cells in particular.

Specific IgG antibodies were below protective levels in both clozapine-treated and clozapine-naïve groups (HiB 51.2% vs 55.9%; Pneumococcal 53.7% vs 55.9%; Tetanus 12.2% vs 13.5%)). However, pneumococcal IgA and IgM levels were significantly lower in clozapine-treated patients as compared with clozapine-naïve patients (IgA 31.0 U/L vs 58.4 U/L; IgM 58.5 U/L vs 85 U/L) (p<0.001) (see Table 2).

Mean levels of CSMBs were significantly reduced at 1.87% in clozapine-treated patients referred independently to clinic and not included in the overall study (n=12) and in CVID patients (n=54) as compared with healthy controls (n=36) and the reference range of 6.5-29.1% (p<0.0001) (see FIG. 3A). Mean levels of plasmablasts were also reduced in clozapine-treated patients (p=0.04).

FIG. 3B shows an extension of the data in FIG. 3A in which referred clozapine patients are compared to age matched CVID and health control subjects. The first graph shows that total B cell numbers are similar between clozapine, CVID and healthy controls and the second graph demonstrates no significant difference between clozapine treated and healthy control marginal zone B cell numbers while there is an increased number observed in CVID patients. The lower two graphs show a significant reduction in both CSMB and plasmablasts in both clozapine treated and CVID patients over healthy controls.

### Example 2

### Second Observational Study on Human Patients on Anti-Psychotic Therapy

Using a cross-sectional observational design in patients on anti-psychotic therapy, this study seeks to test the association between clozapine use, immunophenotype—specifically circulating B cell subsets and immunoglobulin levels—and documented infections, in comparison to other anti-psychotic medication. The study is recruiting patients established on clozapine and those on other antipsychotic drugs from Ashworth Hospital and outpatients from community mental health services in Mersey Care NHS Foundation Trust. The findings will partly provide validation of those from the initial observational study in an orthogonal population, in addition to extending insights into the impact of clozapine on B cell populations through more detailed immunophenotypic analysis.

The study entails a single blood test for detailed immunological analysis and completion of a clinical research form-based questionnaire detailing important clinical parameters including documented infection history, past medical history and concurrent medication use. The findings will be analysed to identify any association between clozapine, circulating B cell levels/function and immunoglobulin levels, its frequency and severity, as well as specificity in relation to other antipsychotic medications.

### Study Aims and Objectives

The specific research questions this study seeks to answer are:

### Primary Outcomes:

i) Is chronic treatment with clozapine associated with (a) a higher proportion of those with specific B cell subsets (namely class-switched memory B cells and plasma cells) below reference ranges and (b) a higher proportion of those with circulating immunoglobulin levels (IgG, IgA and IgM) below references compared to proportions below reference range observed in controls?

### Secondary Outcomes:

ii) Is clozapine associated with reductions in specific antibodies (e.g. pneumococcus, tetanus and Hib) compared to controls?
iii) Is clozapine use associated with an effect on circulating T cells (number/function) compared to controls?
iv) Is clozapine associated with a higher frequency of infections and antibiotic use than controls?
v) Are the primary outcomes related to duration of clozapine therapy?

### Immune Biomarkers

The following immune biomarkers are tested:
1. Total IgG IgM, IgA, and serum electrophoresis with immunofixation if appropriate;
2. Specific IgG levels—tetanus toxoid, pneumococcus, Hib (±IgA and IgM for pneumococcus);
3. Detailed immune cell phenotyping through FACS analysis, including:
   a. Lymphocyte phenotypes—(including CD3, CD4, CD8, CD19, CD56)
   b. B cell panel (based on the EUROClass classification of B cell phenotype (Wehr et al., 2008)) which includes CSMB cells and plasmablasts
   c. Naïve T cell panel
4. RNA extraction from PBMCs (whole blood stored in a RNA preservation solution, e.g. Universal container with ^{˜}4-5 mL RNALater or in PAXgene tube to preserve RNA integrity) for subsequent RNA transcription analysis

All immune biomarker samples are processed and analysed in a UKAS Accredited validated NHS laboratory.

### Results

At the time of writing this study is still recruiting but an interim analysis of the available collected immunophenotypic data (approximately ⅔rds of the way through recruitment) has been undertaken with the caveat that this represent a proportion of the final projected sample size (n ^{˜}100).

The major findings so far are detailed below:

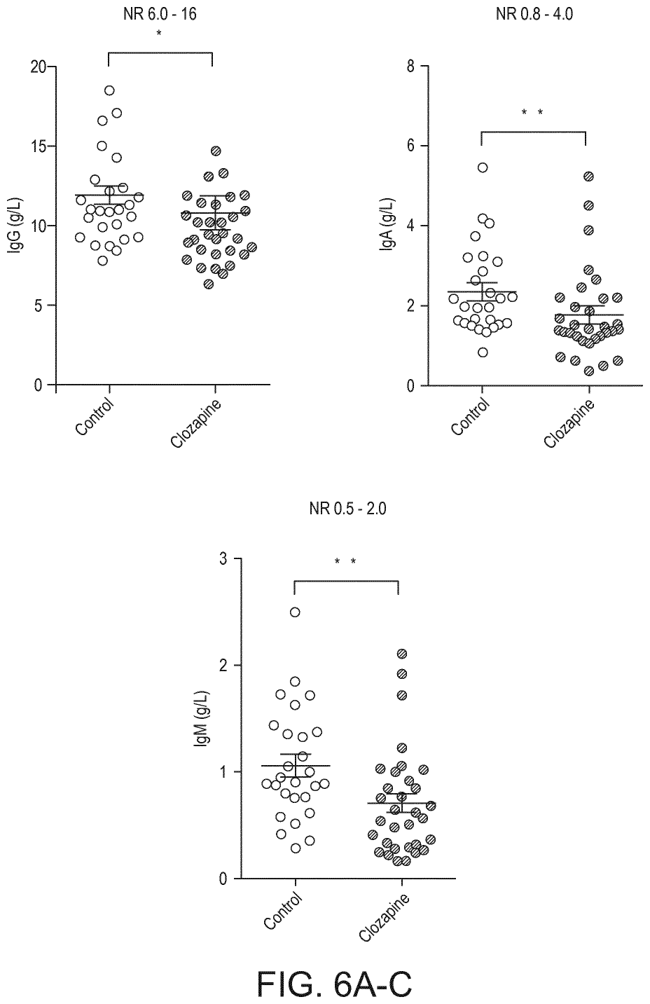

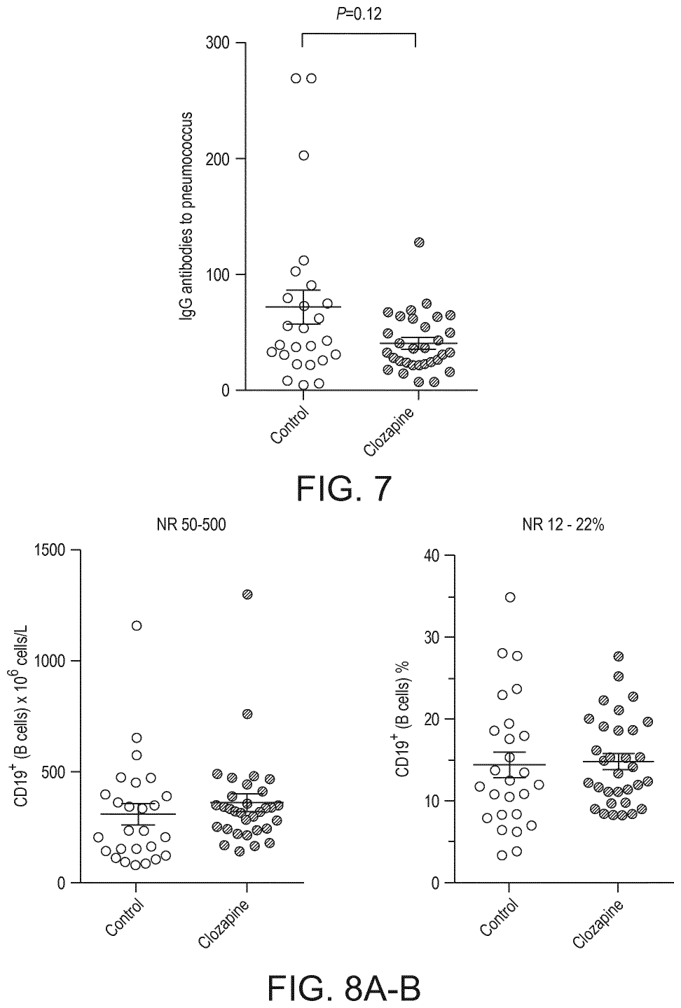

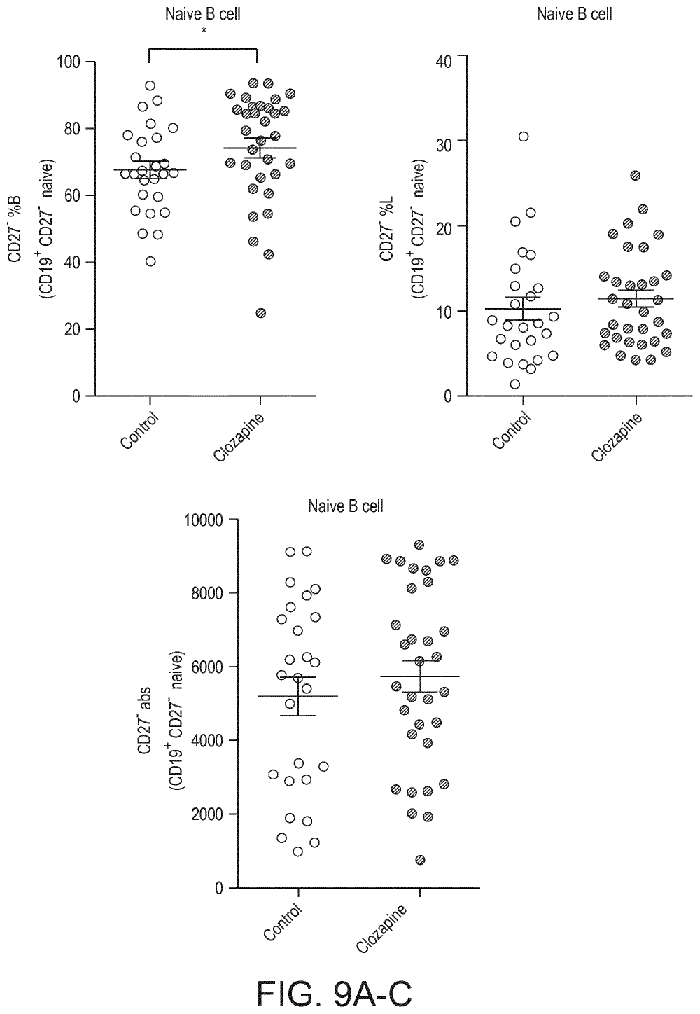

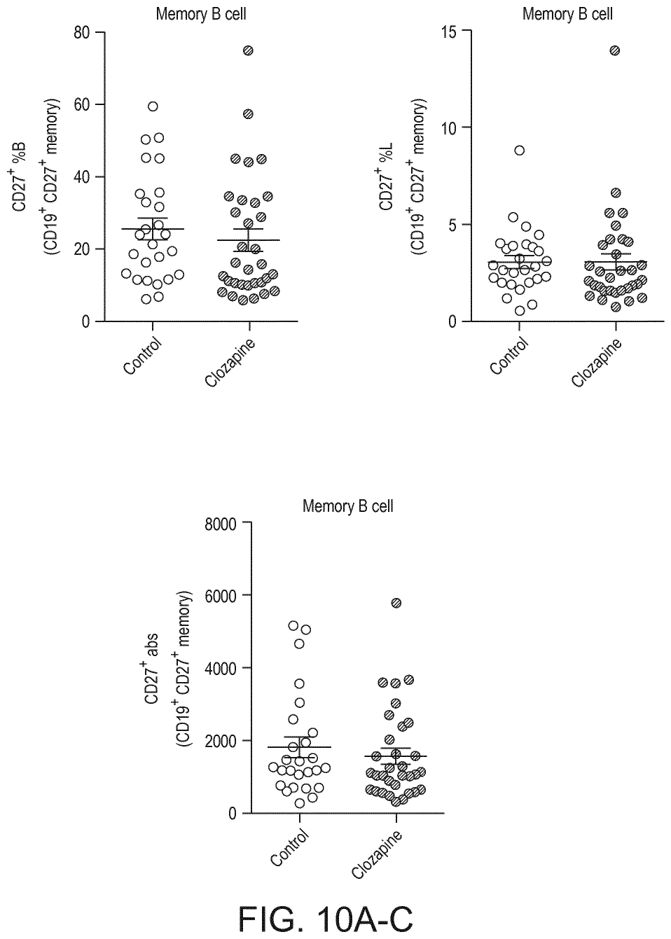

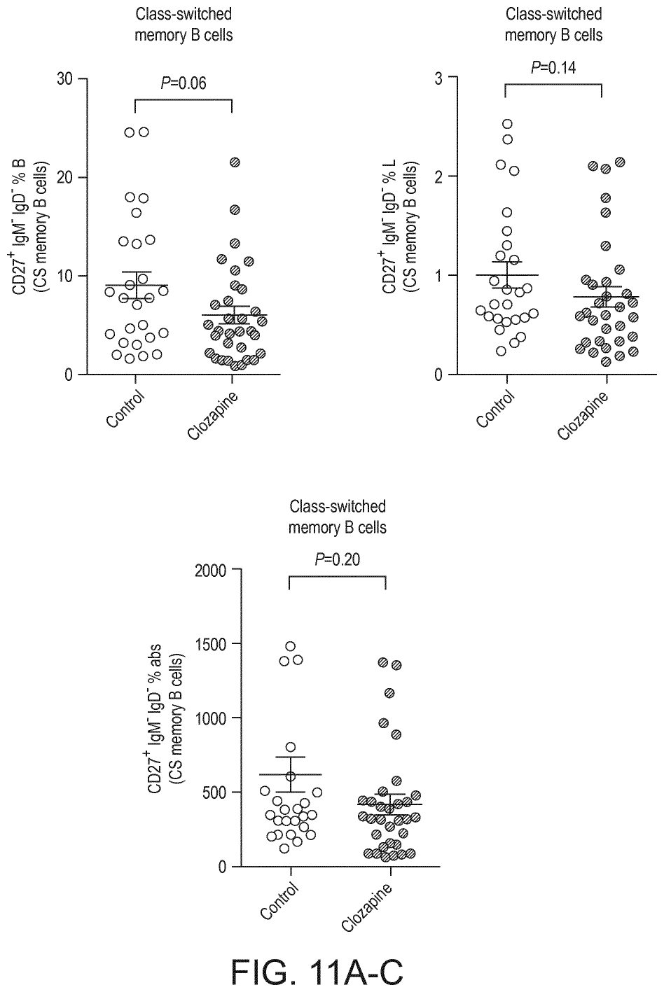

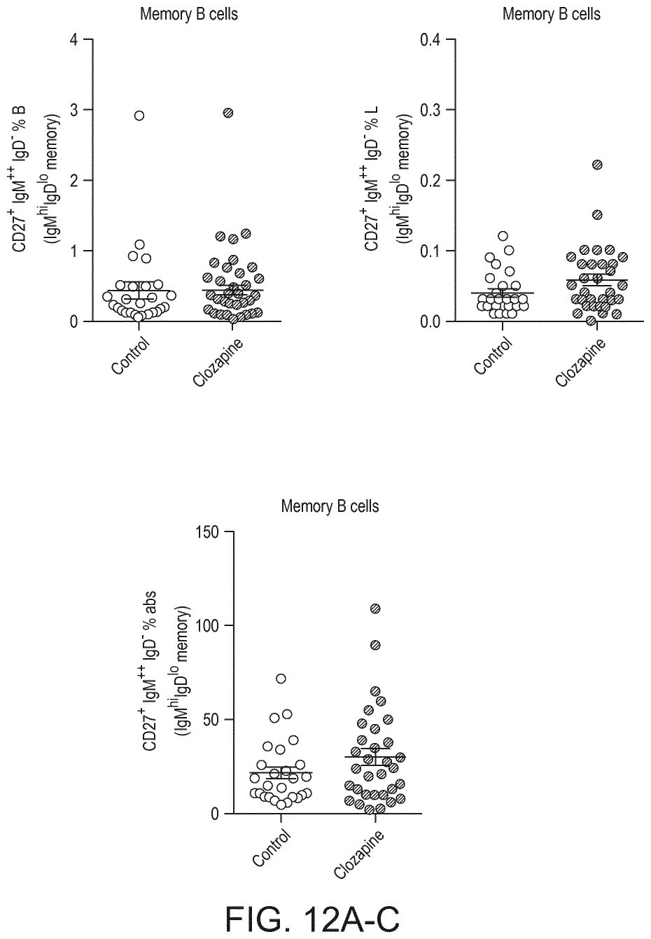

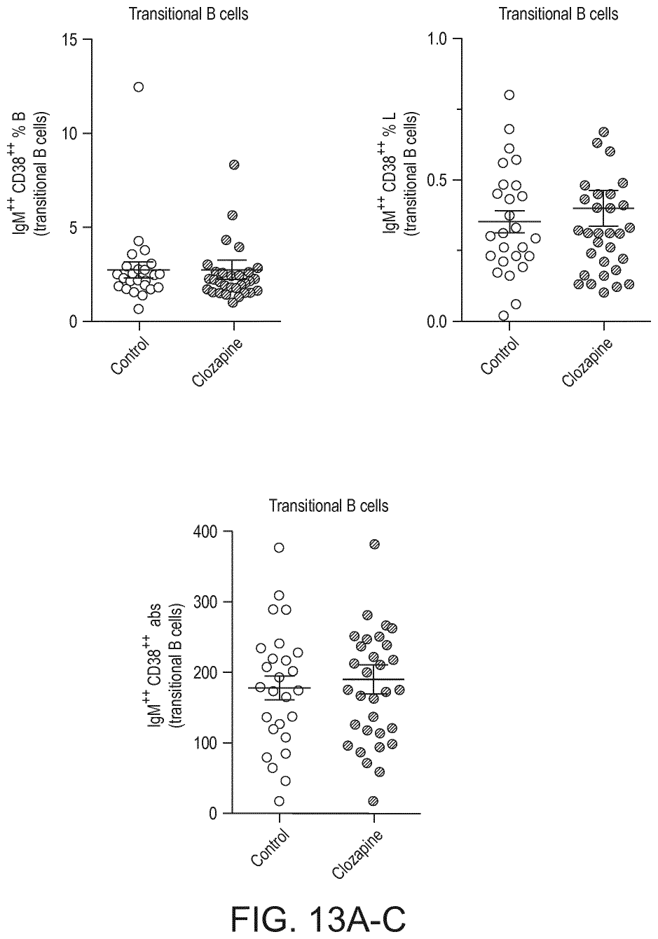

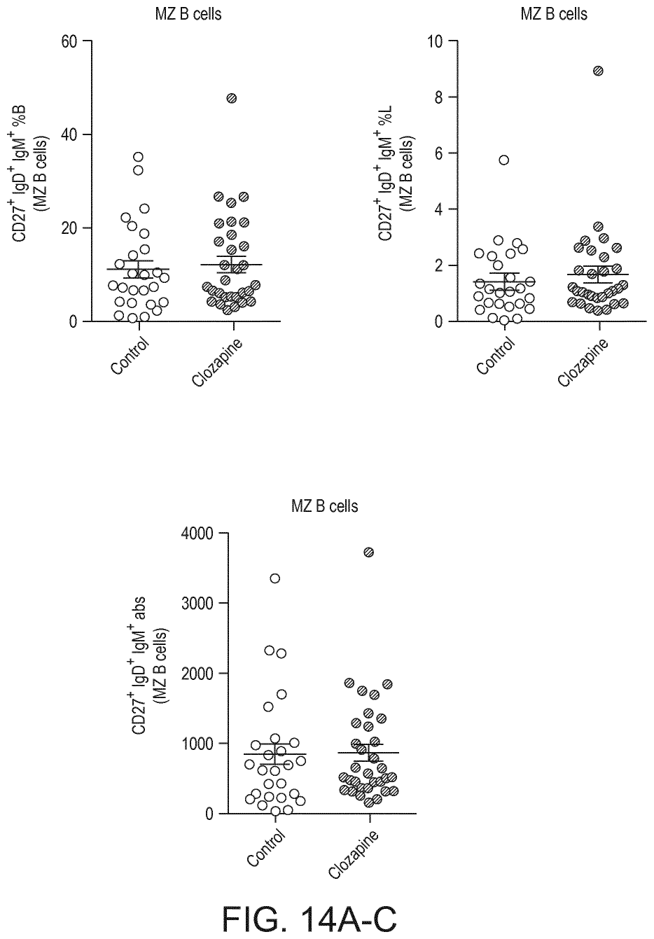

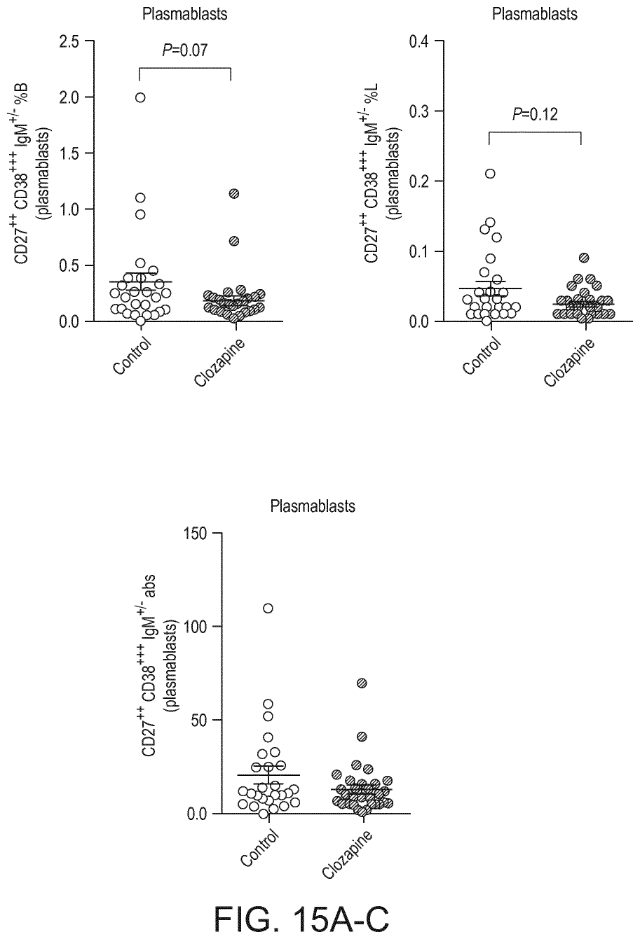

a. Significantly reduced levels of circulating total IgG, IgA and IgM in patients on clozapine versus patients who have never taken clozapine (i.e. control, clozapine naive) (see FIG. 6A-C). These reductions are relatively greater for Ig of the A and M subclass. In addition, a trend to lower IgG antibodies against pneumococcus is present in those treated with clozapine (see FIG. 7).
b. Overall CD19⁺ B cell numbers are not significantly different between groups (see FIG. 8A-B).
c. Small increase in the number of naive (CD19⁺ CD27⁻) B cells expressed as a proportion of total CD19⁺ B cells (see FIG. 9A-C).
d. Strong trends to a specific reduction in class-switched memory B cells (P=0.06 vs control, CD27⁺ IgM⁻ IgD⁻ as % B) in those treated with clozapine (see FIG. 11A-C) without perturbation of the overall memory B cell pool (see FIG. 10A-C) or IgM^{hi} IgD^{lo} memory B cell subpopulation (see FIG. 12A-C).
e. No significant difference between groups in circulating levels of transitional B cells or marginal zone B cells (See FIGS. 13A-C and 14A-C).
f. Strong trends to reduction in levels of plasmablasts in patients treated with clozapine (P=0.07 vs control clozapine naive) (see FIG. 15A-C).

### Example 3

### In Vivo Wild Type Mouse Study—Effect of Clozapine Versus Haloperidol

The impact of clozapine on B cell development, differentiation and function (inferred from circulating immunoglobulin levels) in primary (bone marrow) and secondary (spleen and also mesenteric lymph node) lymphoid tissue in wild type mice in the steady state (i.e. in the absence of specific immunological challenge) was assessed.

The specific objectives were to:

a) Determine the impact of clozapine on major B cell subsets in bone marrow and key secondary lymphoid organs (spleen and mesenteric lymph node) of healthy mice.
b) Define whether a dose-response relationship exists for clozapine on aspects of the B cell immunophenotype.
c) Assess the effect of clozapine administration on the circulating immunoglobulin profile of healthy mice.
d) Determine the specificity of clozapine's effect on the above readouts by comparison to another antipsychotic agent.

### Method

### Animals:

Young adult (age 7-8 weeks) C57BL/6 mature female mice were used for the study. Mice were housed at 22° C. in individually ventilated cages with free access to food and water and a 12-h light/dark cycle (8 a.m./8 p.m.). Mice acclimatised for 1 week on arrival prior to initiating experiments.

### Experimental Groups and Dose Selection:

Mice were allocated into one of five experimental groups as follows:

| 1. Control | saline | |
|---|---|---|
| 2. Clozapine low dose | 2.5 | mg/kg |
| 3. Clozapine intermediate dose | 5 | mg/kg |
| 4. Clozapine high dose | 10 | mg/kg |

| | |
|---|---|
| 5. Haloperidol | 1 mg/kg (intermediate dose) |

Dosing was given in staggered batches with each batch containing mice assigned to each experimental arm to reduce bias.

| | | Clozapine | Clozapine | Clozapine 10 | Haloperidol | Mice per |
|---|---|---|---|---|---|---|
| | Control | 2.5 mg/kg | 5 mg/kg | mg/kg | 1 mg/kg | batch |
| Batch 1 | 2 | 2 | 2 | 2 | 2 | 10 |
| Batch 2 | 2 | 2 | 2 | 2 | 2 | 10 |
| Batch 3 | 2 | 2 | 2 | 2 | 2 | 10 |
| Batch 4 | 2 | 2 | 2 | 2 | 2 | 10 |
| Batch 5 | 2 | 2 | 2 | 2 | 2 | 10 |
| Batch 6 | 2 | 2 | 2 | 2 | 2 | 10 |
| Mice per | 12 | 12 | 12 | 12 | 12 | 60 |
| group | | | | | | |

Dose selection was initially based on a literature review of studies administering these drugs chronically to mice (Ishisaka et al., 2015; Li et al., 2016a; Mutlu et al., 2012; Sacchi et al., 2017; Simon et al., 2000; Tanyeri et al., 2017), the great majority of which had employed the intraperitoneal (IP) route of administration: clozapine (1.5, 5, 10, 25 mg/kg/day) (Gray et al., 2009; Moreno et al., 2013); haloperidol (0.25 mg/kg, 1 mg/kg/day) (Gray et al., 2009) and taking into account the LD50 for both drugs (clozapine 200 mg/kg, haloperidol 30 mg/kg).

Subsequently, pilot studies were undertaken to assess the impact of these, particularly of the higher doses of clozapine, to refine dose selection and maximise the welfare of treated mice. Clear dose-related sedative effects were evident from dosages of clozapine starting at 5 mg/kg, with marked psychomotor suppression (with respect to depth and duration) observed at the highest doses assessed (20 mg/kg and 25 mg/kg). In addition, effects on thermoregulation were also evident, necessitating use of a warming chamber and general supportive measures to defend thermal homeostasis. These adverse effects were consistent with the known (on-target) profile of clozapine in preclinical (Joshi et al., 2017; McOmish et al., 2012; Millan et al., 1995; Williams et al., 2012) and clinical settings (Marinkovic et al., 1994), with tolerance developing after the initial few days of dosing, as has been described in humans (Marinkovic et al., 1994).

Mice (n=12/group) were treated by once daily IP injection of the respective control solution/clozapine/haloperidol for 21 consecutive days.

### Biological Samples for Immunophenotyping:

At the end of the experimental period, mice were humanely euthanised and blood samples obtained for serum separation, storage at −80° C. and subsequent measurement of immunoglobulin profiles (including the major immunoglobulin subsets IgG1, IgG2a, IgG2b, IgG3, IgA, IgM, and both light chains kappa and lambda) by ELISA.

In parallel, tissue samples were rapidly collected from bone marrow (from femur), spleen and mesenteric lymph nodes for evaluation of cellular composition across these compartments using multi-laser flow cytometric detection and analysis.

### B Cell Immunophenotyping by Flow Cytometry:

Focused B cell FACS (fluorescence-activated cell sorter) panels were prepared separately for both primary (bone marrow) and secondary (spleen/lymph node) lymphoid tissue to allow an evaluation of drug impact on the relative composition of B cell subsets spanning the spectrum of antigen-independent and -dependent phases of B cell development.

Individual antibodies employed for flow cytometry panels were pilot tested in the relevant tissues (i.e. bone marrow, spleen and mesenteric lymph node) and the optimal dilution of each antibody determined to enable clear identification of subpopulations. FACS data were extracted by BD FACSymphony and analysed by FlowJo software.

### Results

### Body Weight:

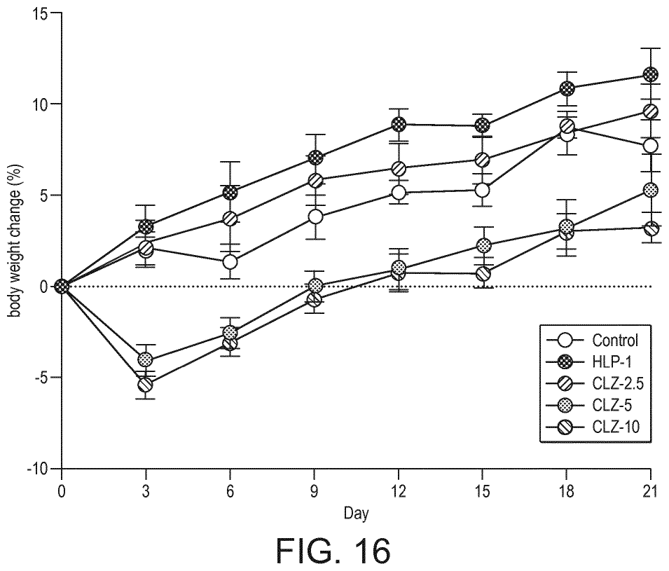

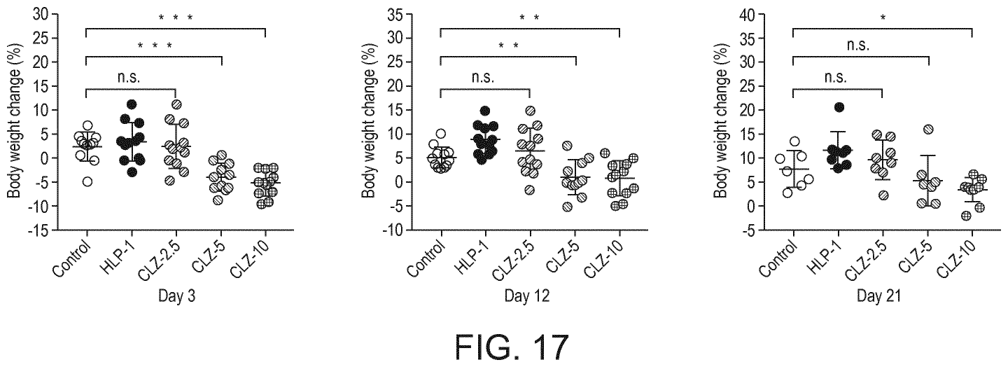

Clozapine (CLZ) induced a transient fall in body weight at both 5 mg/kg and 10 mg/kg doses, maximal by 3 days but recovering fully to baseline by day 9 with progressive weight gain beyond this (see FIGS. 16 and 17). This finding is likely to reflect the sedative effect of clozapine on fluid/food intake during the initial few days of dosing, with evidence of tolerance to this emerging over the course of the experiment.

### Early B Cell Development in Bone Marrow:

B cells originate from hematopoietic stem cells (HSCs), multipotent cells with self-renewal ability, located in the bone marrow. This early B cell development occurs from committed common lymphoid progenitor cells and progresses through a set of stages, dependent on physical and soluble chemokine/cytokine interactions with bone marrow stromal cells, defined using cell surface markers.

The earliest B cell progenitor is the pre-pro-B cell, which expresses B220 and has germline Ig genes. Next, pro-B cells rearrange their H (heavy) chain Igμ genes, and express CD19 under the control of transcription factor Pax5. At the pre-B cell stage, cells downregulate CD43, express intracellular Igμ, and then rearrange the L (light) chain and upregulate CD25 in an Irf4-dependent manner.

Successfully selected cells become immature (surface IgM⁺IgD⁻) B cells. Immature B cells are tested for autoreactivity through a process of central tolerance and those without strong reactivity to self-antigens exit the bone marrow via sinusoids to continue their maturation in the spleen.

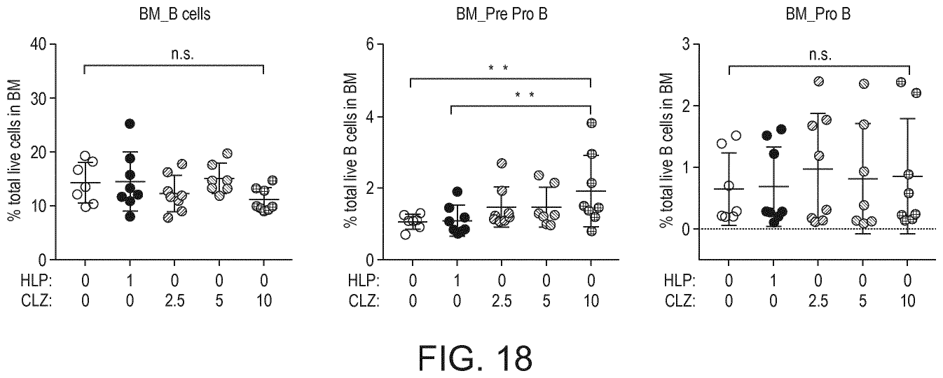

No overall reduction in B cells in the bone marrow (BM) was observed at any dose of clozapine (see FIG. 18). However, a significant increase in the proportion of very early B cell progenitors, the pre-pro B cells (i.e. B220⁺CD19⁻CD43⁺CD24^{lo}BP-1⁻IgM⁻IgD⁻) was observed with 10 mg/kg clozapine, without any change evident in the subsequent pro-B cell fraction (see FIG. 18). In contrast, no significant effect of haloperidol was evident on any of these early developing B cell subsets.

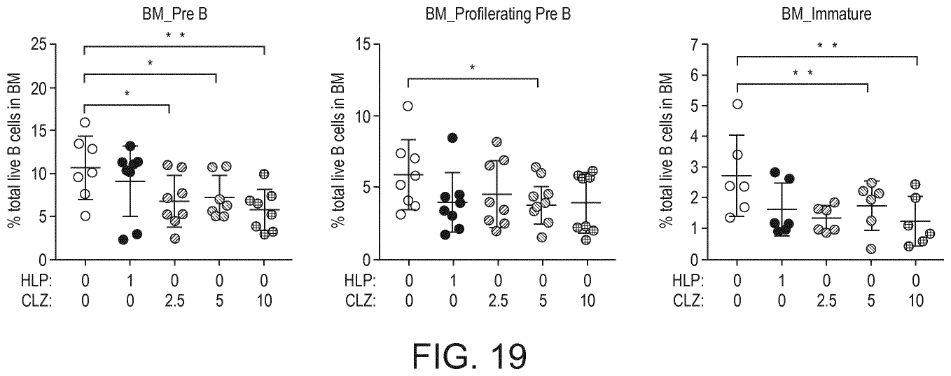

Examination of subsequent stages of B cell development in bone marrow revealed a reduction in pre-B cells (i.e. B220⁺CD19⁺CD43⁻CD24⁺BP-1⁻IgM⁻IgD⁻) in mice treated with clozapine (see FIG. 19). Notably this effect exhibited dose-dependency, with a significant difference observed verses control mice with even the lowest dose of clozapine employed (2.5 mg/kg). Furthermore, the percentage of pre-B cells that were proliferating (i.e. B220⁺CD19⁺CD43⁻CD24^{hi}BP-1⁺IgM⁻IgD⁻) was diminished with clozapine, reaching significance for the 5 mg/kg dose (see FIG. 19). Correspondingly, a reduction in the percentage of immature B cells in bone marrow was identified (i.e. B220⁺CD19⁺CD43⁻ CD24⁺IgM⁺IgD⁻) (see FIG. 19).

Together, these findings suggest a specific impact of clozapine on early B cell development, with a modest arrest between the pre-pro-B cell and pre-B cell stages in the absence of specific immunological challenge.

### Peripheral B Cell Development—Total Splenic B Cells:

After emigrating from the bone marrow, functionally immature B cells undergo further development in secondary lymphoid organs, enabling further exposure to (peripheral) self-antigen and peripheral tolerance (resulting in cell deletion through apoptosis, anergy or survival). The majority of immature B cells exiting bone marrow do not survive to become fully mature B cells, a process regulated by maturation and survival signals received in lymphoid follicles, including BAFF (B cell activating factor) secreted by follicular dendritic cells.

Mice treated with clozapine at 5 mg/kg and 10 mg/kg were seen to have a significantly lower percentage of splenic B cells (i.e. B220⁺TCR-β⁻) expressed as a proportion of total live splenocytes (see FIG. 21). No effect was identified on other cell populations (i.e. B220⁻TCR-β⁻), which may include γδ T cells (which do not express the αβ T cell receptor, TCR), natural killer (NK) cells, or other rare lymphoid cell populations (see FIG. 21). This was accompanied by a reciprocal increase in the percentage of splenic T cells (i.e. B220−TCR-β+) (see FIG. 21). In contrast, activated T cells (i.e. B220⁺TCR-β⁺), reflecting a small proportion of total live splenocytes were reduced in dose-dependent fashion by clozapine compared to control, an effect also modestly apparent for haloperidol (see FIG. 21).

These findings suggest that clozapine, but not haloperidol, is able to affect peripheral (splenic) B cells in addition to the observed changes in bone marrow B cell precursors.

### Splenic B Cell Subpopulations:

Immature B cells exiting the bone marrow and entering the circulation are known as transitional B cells. These immature cells enter the spleen and competitively access splenic follicles to differentiate via transitional stages to immunocompetent naive mature B cells. This occurs sequentially in the follicle from transitional type 1 (T1) cells, similar to immature B cells in bone marrow, to type 2 (T2) precursors. The latter are thought to be the immediate precursor of mature naive B cells. T2 B cells have been demonstrated to show greater potency in response to B cell receptor stimulation than T1 B cells, suggesting that the T2 subset may preferentially undergo positive selection and progression into the long-lived mature B cell pool (Petro et al., 2002).

Transitional cells can differentiate into follicular B cells, representing the majority of peripheral B cells residing in secondary lymphoid organs, or a less numerous population, marginal zone (MZ) B cells residing at the white/red pulp interface which are able to respond rapidly to blood-borne antigens/pathogens.

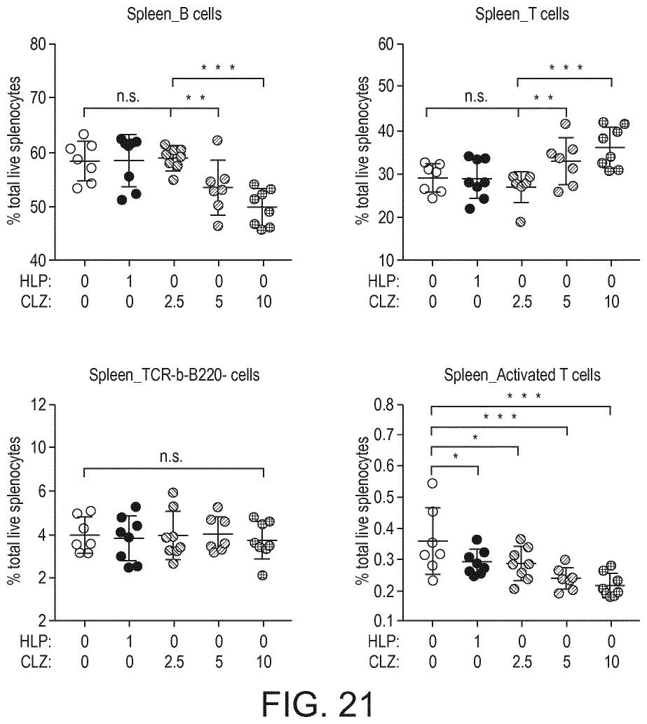

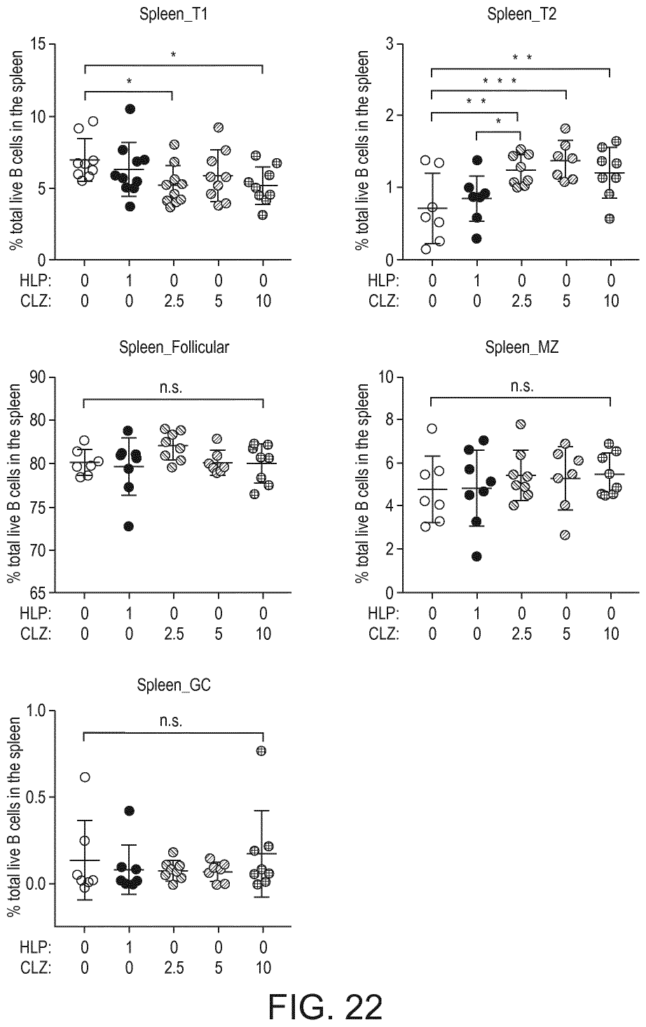

Mice treated with clozapine were found to have a mildly reduced proportion of newly emigrated transitional stage 1 (T1) B cells in the spleen, including at the 2.5 mg/kg dose, which may in part reflect the reduction in percentage of bone marrow immature B cells (see FIG. 22). In contrast, a small increase in the proportion of T2 B cells was identified across all doses of clozapine (see FIG. 22), consistent with enhanced positive selection of T1 B cell subsets for potential progression into the long-lived mature B cell pool.

While clozapine administration reduced the splenic B cell contribution to live splenocytes (see FIG. 21), no specific reductions were identified in either splenic follicular (i.e. B220⁺CD19⁺CD21^{mid}CD23⁺) or marginal zone (i.e. B220⁺CD19⁺CD21⁺CD23^{Lo/-}) B cell subsets (see FIG. 22), suggesting that in the immunologically unchallenged state, clozapine administration in mice results in a global reduction in splenic B cell populations.

Germinal centres (GCs) are micro-anatomical structures which form over several days in B cell follicles of secondary lymphoid tissues in response to T cell-dependent antigenic (e.g. due to infection or immunisation) challenge (Meyer-Hermann et al., 2012). Within GCs, B cells undergo somatic hypermutation of their antibody variable regions, with subsequent testing of the mutated B cell receptors against antigens displayed by GC resident follicular dendritic cells. Through a process of antibody affinity maturation, mutated B cells which higher affinity to antigen are identified and expanded. In addition, class switch recombination of the immunoglobulin heavy chain locus of mature naive (IgM⁺IgD⁺) B cells occurs before and during GC reactions, modifying antibody effector function but not its specificity or affinity for antigen. This results in isotype switching from IgM to other immunoglobulin classes (IgG, IgA or IgE) in response to antigen stimulation.

GCs are therefore sites of intense B cell proliferation and cell death, with outcomes including apoptosis, positive selection for a further round of somatic hypermutation (i.e. cyclic re-entry), or B cell differentiation into antibody secreting plasma cells and memory B cells (Suan et al., 2017). In the steady state, GC cells (i.e. B220+CD19+IgD−CD95+GL-7+) formed a very small proportion of total live B cells in the spleen, with no differences observed versus control or haloperidol in response to clozapine administration (see FIG. 22).

### Bone Marrow Antibody Secreting Cell Populations:

Antibody secreting cells represent the end-stage differentiation of the B cell lineage and are widely distributed in health across primary and secondary lymphoid organs, the gastrointestinal tract and mucosa (Tellier and Nutt, 2018). These cells all derive from activated B cells (follicular, MZ or B1). Plasmablasts, representing short-lived cycling cells, can be derived from extra-follicular differentiation pathway in a primary response (producing relatively lower affinity antibody), as well as from memory B cells that have undergone affinity maturation in the GC (Tellier and Nutt, 2018).

Plasmablasts developing in GCs can leave the secondary lymphoid organ and home to the bone marrow. Here, only a small proportion are thought to be retained and establish themselves in dedicated micro-environmental survival niches to mature into long-lived plasma cells (Chu and Berek, 2013), a process thought to be regulated by docking onto mesenchymal reticular stromal cells (Zehentmeier et al., 2014) and requiring haematopoietic cells (e.g. eosinophils) (Chu et al., 2011a), the presence of B cell survival factors (e.g. APRIL and IL-6) (Belnoue et al., 2008) and hypoxic conditions (Nguyen et al., 2018).

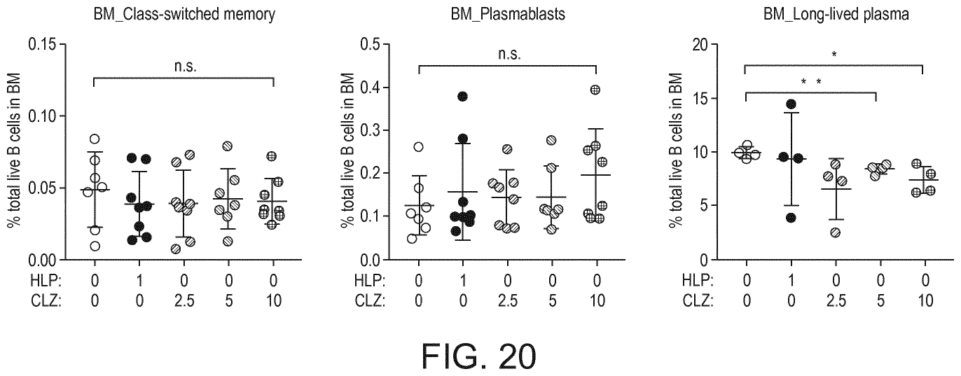

In the healthy state, the bone marrow houses the majority of long-lived plasma cells. Clozapine at 5 and 10 mg/kg induced a significant reduction in the percentage of long-lived plasma cells in the bone marrow (i.e. B220^{lo} CD19⁻IgD⁻IgM⁻CD20⁻CD38⁺⁺CD138⁺) by ^{˜}30% compared to control (see FIG. 20). In contrast, no effect of haloperidol was seen on this specific B cell population (see FIG. 20). No significant changes were detected in either class-switched memory B cells (i.e. B220⁺CD19⁺CD27⁺IgD⁻ IgM⁻CD20⁺CD38^{+/-}) or plasmablasts (i.e. B220^{lo}CD19⁺CD27⁺IgD⁻IgM⁻CD20⁻CD38⁺⁺) in the bone marrow with any treatment, however both these represent a very small proportion of total B cells in the bone marrow in the immunologically unchallenged steady state (see FIG. 20).

These findings indicate that clozapine can exert a specific effect to reduce the proportion of long-lived plasma cells in the bone marrow, a population thought to be the major source of stable antigen-specific antibody titres in plasma involved in humoral immune protection and, in pathogenic states, stable autoantibody production.

### Circulating Immunoglobulin Levels:

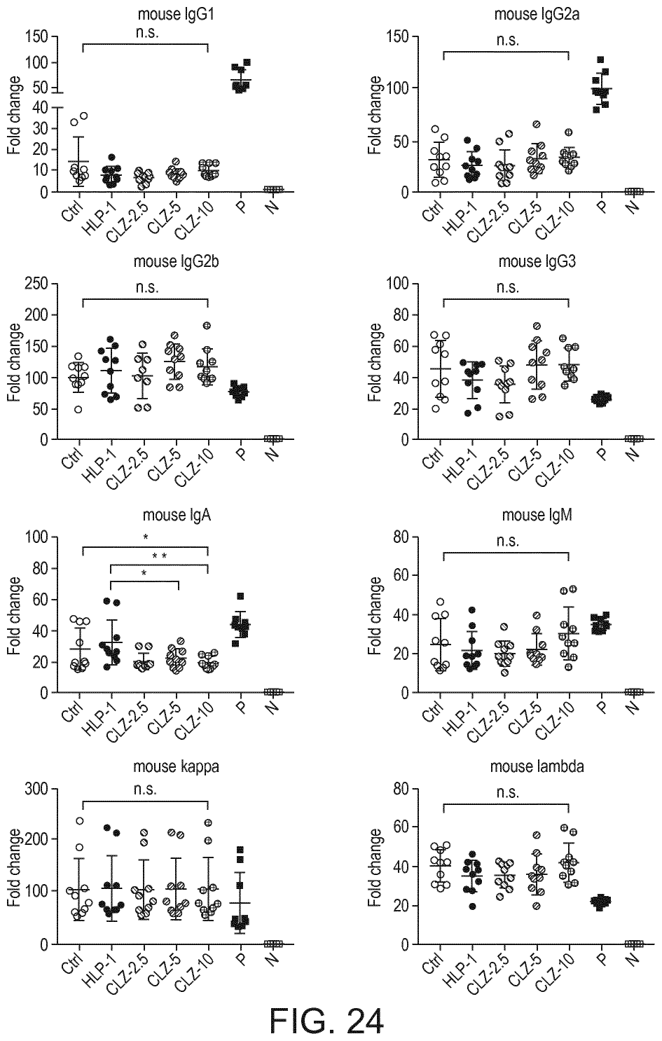

Clozapine administration at both 5 and 10 mg/kg resulted in a reduction in circulating IgA levels compared to control, an effect not observed with haloperidol (see FIG. 24; P, positive control; N, negative control). No other isotype classes were affected under the experimental conditions used (see FIG. 24).

### Mesenteric Lymph Nodes:

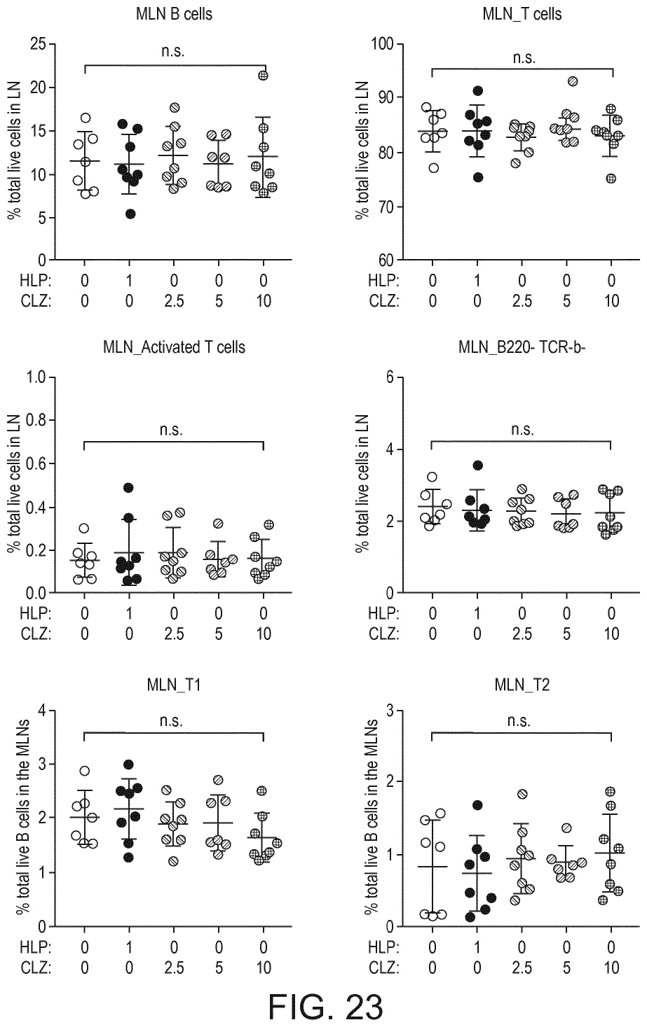

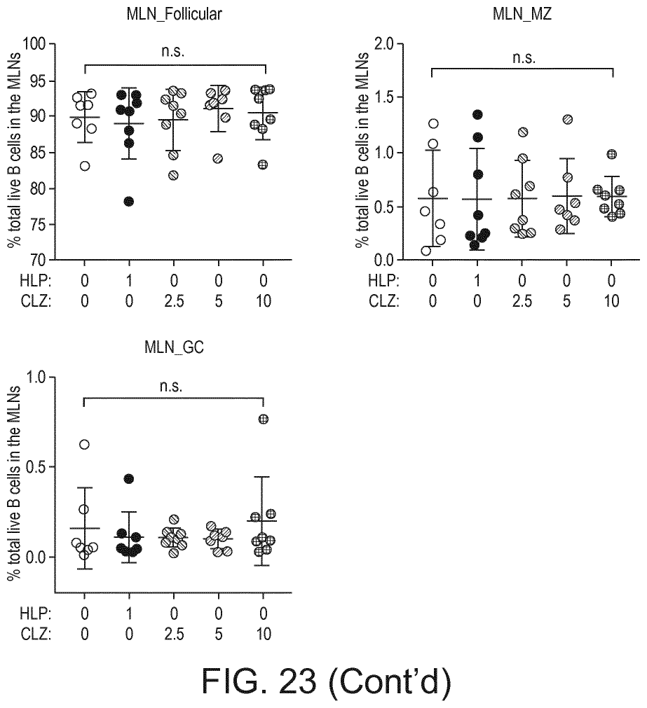

Under the current experimental conditions, no significant differences were identified between any of the groups in lymphocyte subpopulations assessed in mesenteric lymph nodes (MLN) (see FIG. 23).

### Conclusion

This study investigated the potential for clozapine to influence the immunophenotype of wild type mice in the steady state, specifically B cell subpopulations, with functional impact inferred through circulating levels of immunoglobulins. The major findings of this study are that 3 weeks parenteral (I.P.) administration of clozapine:

a) Increases the proportion of pre-pro-B cells while reducing the proportion of later-stage pre-B cells and immature B cells in the bone marrow.
b) Reduces the proportion of live splenocytes that are B cells.
c) Exerts subtle effects on developing B cells in the spleen, specifically transitional B cell populations in favouring a greater proportion of T2 type cells.
d) Significantly reduces the proportion of long-lived plasma cells in the bone marrow.
e) Impacts on circulating immunoglobulin levels, specifically lowering IgA.
f) Results in a dose-dependent decrease in the proportion of activated T cells in spleen which, in contrast to all the above findings, was also observed with the dose of haloperidol used.

Taken together, these observations indicate that clozapine exerts complex effects on B cell maturation in vivo, not limited to the late stages of B cell differentiation or activation. Specifically, the findings suggest that clozapine can influence the maturation of early B cell precursors, with a partial arrest of antigen-independent B cell development in the bone marrow.

In parallel, clear effects of clozapine are identified on peripheral B cell subpopulations, with a notable impact on reducing the overall B cell proportion of live splenocytes, and on long-lived antibody secreting plasma cells in the bone marrow. An impact on antibody secreting cells is likely to underlie the observed significant reduction in circulating IgA, particularly striking given the otherwise immunologically unchallenged state of the mice.

Notably, the impact on B cell subpopulations was not observed with a comparator antipsychotic agent, haloperidol, consistent with specificity of action of clozapine on B cell maturation. While the current experiments do not enable a distinction between a direct or indirect effect of clozapine on bone marrow, peripheral and late B cell populations, taken together with findings from separate in vitro B cell proliferation assays, an indirect effect is deemed more likely. This may involve a variety of other myeloid, lymphoid (e.g. T follicular helper cells) and/or (mesenchymal) stromal supportive cells.

### Example 4

### Mouse Collagen-Induced Arthritis (CIA) Model Study—Effect of Clozapine

The CIA model is a well-established experimental model of autoimmune disease. The inventors have employed the CIA model as a highly clinically relevant experimental system in which B cell-derived pathogenic immunoglobulin made in response to a sample specific antigen drives autoimmune pathology to explore the potential efficacy of clozapine and its associated cellular mechanisms.

### Method

### Animals:

Adult (age 13-15 weeks) DBA/1 male mice were purchased from Envigo (Horst, Netherlands). Mice were housed at a 21° C.±2° C. in individually ventilated cages with free access to food and water and a 12-h light/dark cycle (7 am/7 pm). Mice were acclimatised for 1 week on arrival prior to initiating experiments.

### Experimental Groups and Dose Selection:

Mice were allocated into one of five experimental groups as follows:

1. Control saline
2. Clozapine 5 mg/kg treatment from day 15 after immunization
3. Clozapine 10 mg/kg treatment from day 15 after immunization
4. Clozapine 5 mg/kg treatment from day 1 after immunization
5. Clozapine 10 mg/kg treatment from day 1 after immunization

Mice (n=10/group) were treated by once daily IP injection of the respective control solution/clozapine until day 10 after onset of clinical features of arthritis. All experiments were approved by the Clinical Medicine Animal Welfare and Ethical Review Body (AWERB) and by the UK Home Office.

### Anti-Arthritic Effect of Clozapine In Vivo:

DBA/1 mice were immunised with bovine type II collagen in CFA and monitored daily for onset of arthritis. Clozapine was administered daily by intraperitoneal injection at doses of 5 mg/kg or 10 mg/kg. Controls received vehicle (saline) alone. Treatment of mice commenced in one experiment on day 1 after immunisation and in a second experiment on day 15 after immunisation. Clinical scores and paw-swelling were monitored for 10 days following onset of arthritis. A clinical scoring system was used as follows. Arthritis severity was scored by an experienced, non-blinded investigator as follows: 0=normal, 1=slight swelling and/or erythema, 2=pronounced swelling, 3=ankylosis. All four limbs were scored, giving a maximum possible score of 12 per animal.

At the end of the experimental period, mice were humanely euthanised and bled by cardiac puncture to obtain blood samples for serum separation, storage at −80° C. and subsequent measurement of specific anti-collagen immunoglobulin (IgG1 and IgG2a isotypes) by ELISA. In parallel, spleen and inguinal lymph nodes were harvested for evaluation of cellular composition across these compartments using multi-laser flow cytometric detection and analysis. Numbers of B cell subsets in spleen and lymph nodes were determined by FACS.

### Statistical Analysis:

Data were analyzed by one-way ANOVA with Tukey's or Dunnett's multiple comparison test or two-way ANOVA with Tukey's multiple comparison test as appropriate. All calculations were made using GraphPad Prism software. A P value less than 0.05 was considered significant.

### Results

### Effect of Clozapine on Onset, Clinical Score and Paw-Swelling:

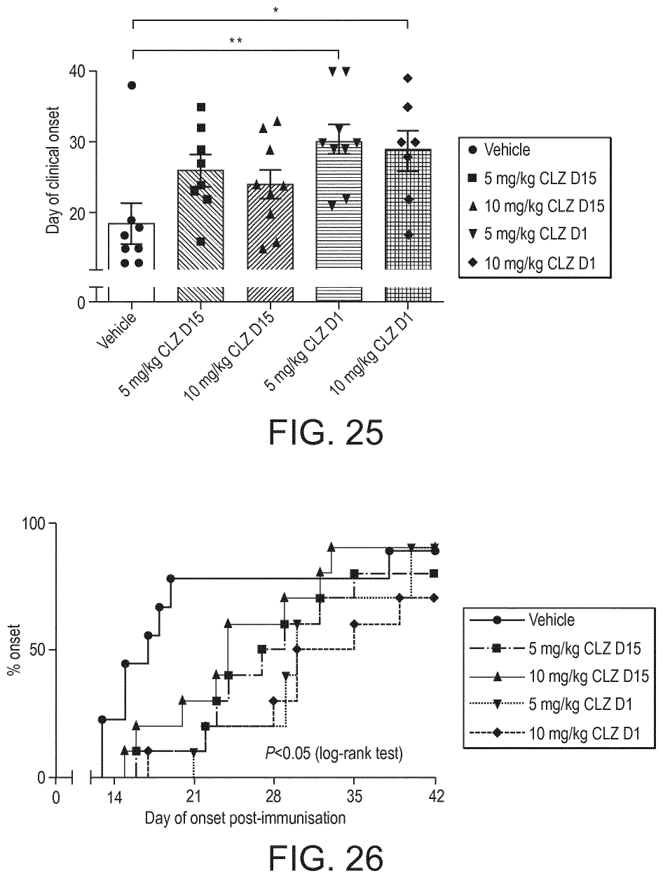

Treatment of mice with clozapine was significantly effective in delaying the onset of arthritis post-immunisation (see FIGS. 25 and 26). In particular, treatment with both doses of clozapine from day 1 was extremely effective in delaying arthritis onset (see FIGS. 25 and 26).

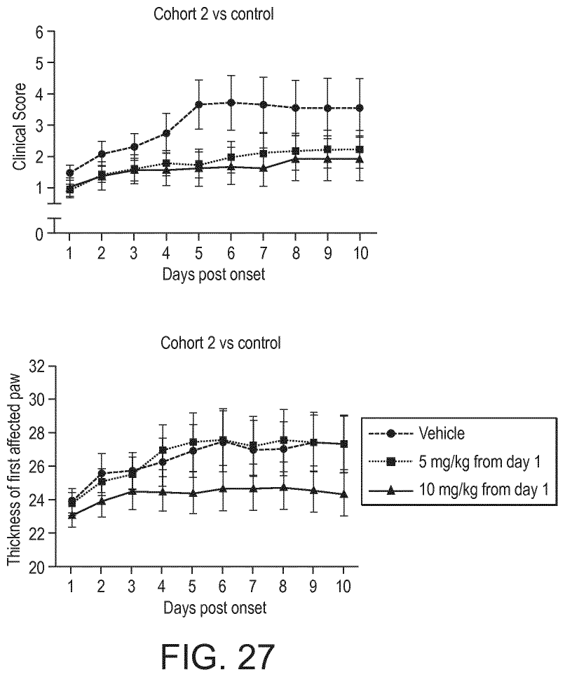

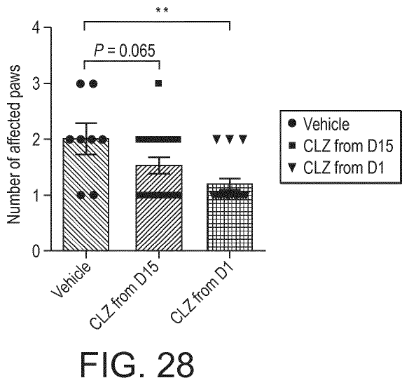

Furthermore, treatment with both doses of clozapine reduced overall clinical score when administered on day 1 and, in the case of 10 mg/kg clozapine, also reduced swelling of the first affected paw (see FIG. 27). Clozapine administration also reduced the total number of affected paws compared to vehicle control, an effect significant with dosing at D1 (see FIG. 28).

### Effect of Clozapine on Peripheral B Cell Subsets:

Mice treated with clozapine at all doses and time points (i.e. 5 mg/kg or 10 mg/kg from day 1 or day 15) were seen to have a significantly lower percentage of B220⁺ B cells in lymph nodes (see FIG. 29). In addition, clozapine administered at 10 mg/kg from day 1 also significantly reduced the proportion of B220⁺ B cells in spleen.

Under the experimental conditions employed, no significant effect of clozapine was observed on plasma cell numbers in lymph node, however a significant reduction in the proportion of plasma cells was identified in spleen at a dose of 10 mg/kg clozapine given on day 1, with nominally lower values for plasma cells as a proportion of live cells at every other dose/time evaluated compared to control (see FIG. 30).

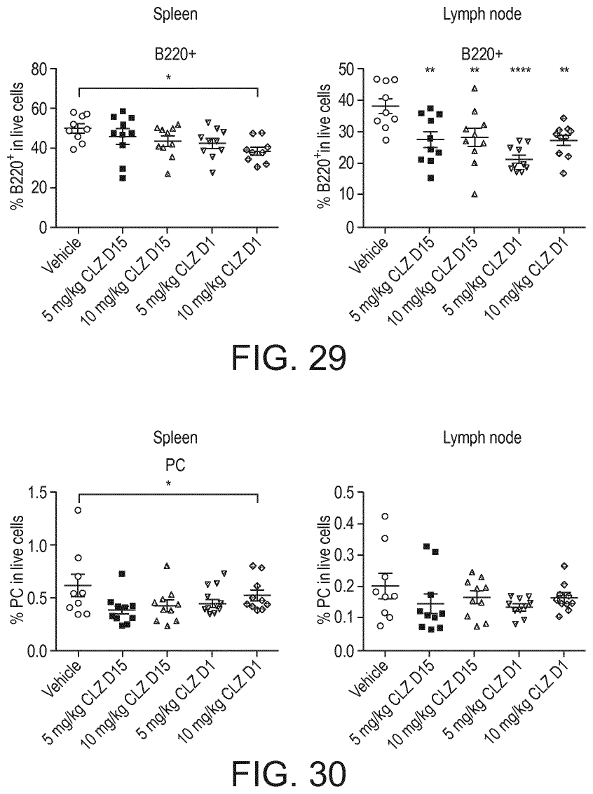

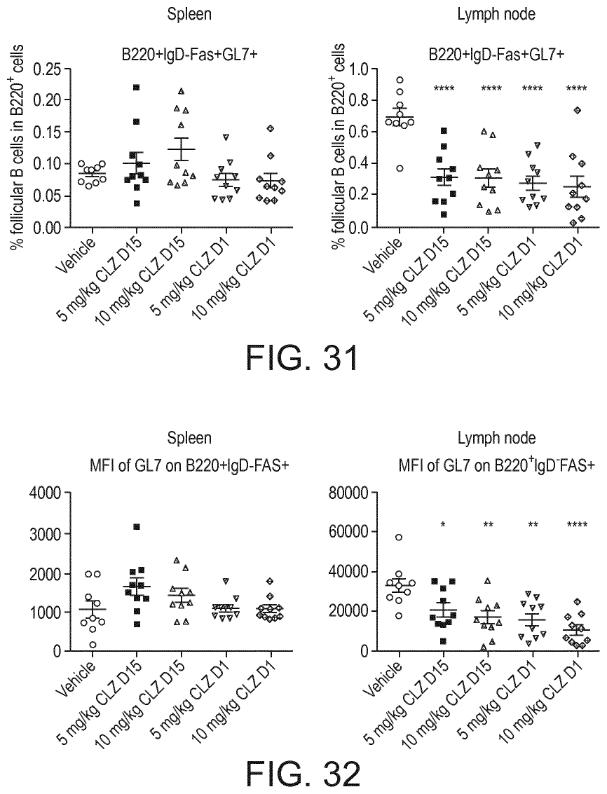

Strikingly significant reductions in lymph node follicular B cells (B220⁺IgD⁻Fas⁺GL7^{hi}) were observed in mice treated with clozapine across all doses/both time points (see FIG. 31). In addition, the level of GL7 expression on follicular B cells in lymph node were significantly decreased across all clozapine treatment groups compared to vehicle treated controls (see FIG. 32). There was evidence of dose- and time-dependency of effect with particularly profound reductions in GL7 expression in mice treated with clozapine from day 1 (see FIG. 32).

### Effect of Clozapine on Anti-Type II Collagen IgG Isotypes:

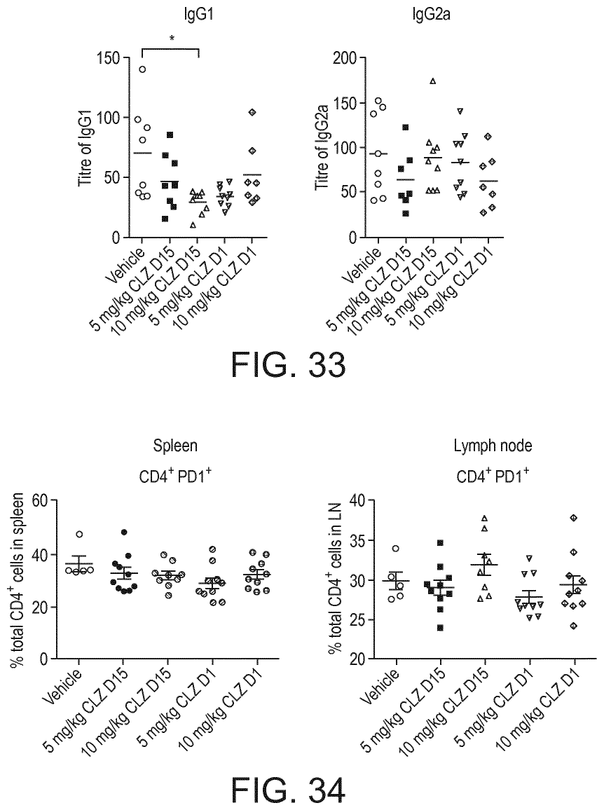

Clozapine administration at 5 or 10 mg/kg from day 1 or day 15 had no significant impact on serum IgG2a measured at a single time point. However, clozapine administration led to nominal reductions in levels of IgG1 across all doses tested, reaching statistical significance for the group treated with 10 mg/kg from day 15 (see FIG. 33).

### Effect of Clozapine on T Follicular Helper Cells:

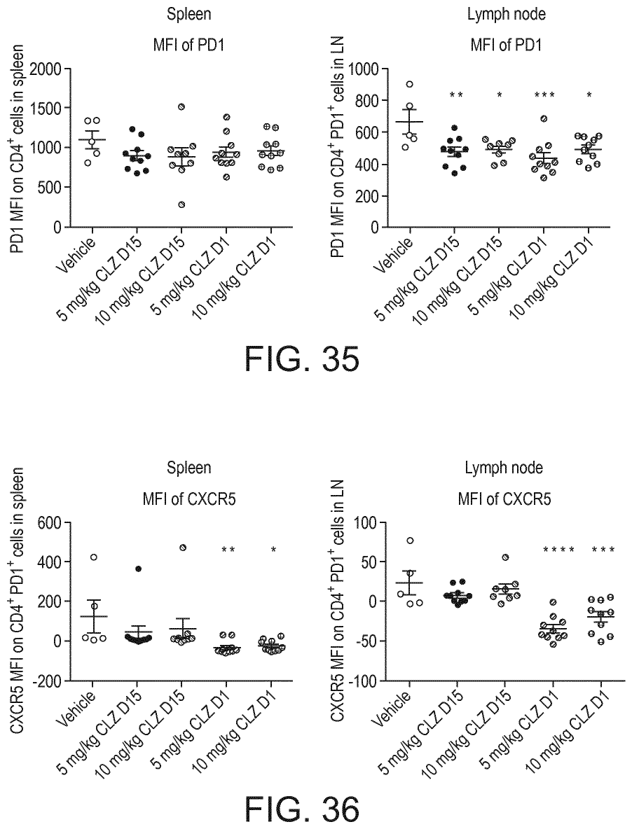

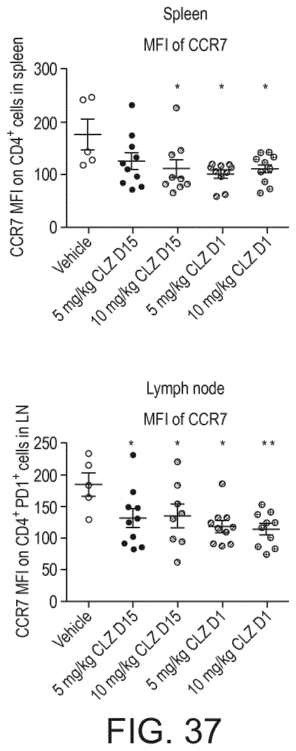

Treatment of mice with 5 mg/kg or 10 mg/kg of clozapine from day 1 or day 15 did not significantly affect proportions of CD4⁺PD1⁺CXCR5⁺ T follicular helper cells in lymph node or spleen (see FIG. 34). However, analysis of mean fluorescence intensity (MFI) revealed robust reductions in expression of PD-1 and CXCR5 on T follicular helper cells in mice-treated with clozapine (see FIGS. 35 and 36). Reduced expression of PD-1 in lymph node T follicular helper cells was evident for clozapine at all doses and time points evaluated (see FIG. 35). In the case of CXCR5 expression, significant reductions were observed in mice dosed with clozapine from day 1 and evident in both lymph node (strongest signal for reduction) and spleen (see FIG. 36). In addition, a reduction in expression of CCR7 was observed on germinal centre resident T follicular helper cells in both lymph node and spleen of mice treated with clozapine (see FIG. 37).

### Conclusion

This study investigated the potential for clozapine to ameliorate CIA and its impact on major B cell subsets. The major findings of this study are as follows.

a) Clozapine is extremely effective at delaying disease onset in the CIA model.
b) Clozapine ameliorates the severity in CIA.
c) Clozapine reduces the proportion of B220⁺ B cells in both spleen and lymph node.
d) Clozapine reduces the proportion of splenic plasma cells.
e) Clozapine results in substantial reduction in the proportion of lymph node follicular B cells (IgD⁻ Fas⁺GL7^{hi}) in B220⁺ B cells and lowers their expression of GL-7.
f) Clozapine demonstrated some ability to reduce pathogenic immunoglobulin, specifically anti-collagen IgG1 (at a dose of 10 mg/kg dosed from D15 after immunisation) in the context of the experimental conditions assessed (single time point immunoglobulin measurement).
g) Clozapine markedly reduces the expression of PD1 and CXCR5, in addition to CCR7, on lymph node T follicular helper cells (PD1⁺CXCR5⁺) without impacting upon the proportion of cells.

Taken together, these observations indicate that clozapine delayed disease onset, probably through multiple mechanisms likely to involve its impact on (secondary) lymphoid tissue and its ability to form functional germinal centres with subsequent impact on antibody producing B cells.

Specifically, clozapine is seen to reduce germinal centre B cells in local lymph node [marked by expression of GL7 in immunised spleen/lymph node (Naito et al., 2007)] following immunisation. GL7^{hi} B cells exhibit higher specific and total immunoglobulin production in addition to higher antigen-presenting capacity (Cervenak et al., 2001). Thus the observation of a reduction in surface expression of the GL7 epitope with clozapine suggests an impact to lower functional activity of these B cells for producing antibody and presenting antigen.

In parallel, clozapine is seen to affect T follicular helper cells, a critical T cell subset which controls the formation of and coordinates the cellular reactions occurring within germinal centres that is essential for somatic hypermutation, isotype class switching and antibody affinity maturation, differentiating B cells into memory B cells or plasma cells. T follicular helper cells therefore specialise in promoting the T cell-dependent B cell response (Shi et al., 2018). In particular, while not affecting the overall proportion of T follicular helper cells, clozapine is seen to reduce PD1 (programmed cell death-1) expression which is essential for proper positioning of T follicular helper cells through promoting their concentration into the germinal centre from the follicle (Shi et al., 2018). PD1 is also required for optimal production of IL-21 by T follicular helper cells, with PD1-PD-L1 interactions (i.e. the cognate ligand of PD1) between T follicular helper cells and germinal centre B cells aiding the stringency of affinity-based selection.

Furthermore, clozapine was seen to reduce the expression of CXCR5 on T follicular helper cells. CXCR5 (CXC chemokine receptor 5) is regarded as the defining marker for these cells; upregulation of CXCR5 enables relocation to the T/B border and, through attraction to CXCL-13, the B cell zone of lymphoid tissue to allow T follicular helper cells to enter the B cell follicle (Chen et al., 2015). Accordingly, reduced expression of CXCR5 on T follicular helper cells would impede their migration into B cell follicles and thereby reduce their ability to localise and interact with germinal centre B cells. Consistent with this, mice deficient in CXCR5 or selectively lacking CXCR5 on T cells display complete resistance to induction in CIA, in concert with reduced secondary lymphoid germinal centre formation and lower anti-collagen antibody production (Moschovakis et al., 2017).

Clozapine was also found to reduce expression of CCR7 on T follicular helper cells. CCR7 downregulation is regarded as an important mechanism through which activated CD4⁺ T cells overcome T zone chemokines which promote retention in the T zone (Haynes et al., 2007). Importantly, promotion of normal germinal centre responses by T follicular helper cells requires a coordinate upregulation of CXCR5 and downregulation of CCR7 (Haynes et al., 2007). Thus, the balanced expression of CXCR5 and CCR7 is critical to fine tuning of T follicular helper cell positioning and efficient provision of B cell help (Hardtke et al., 2005). The observation that clozapine can influence both CXCR5 and CCR7 expression on T follicular helper cells is therefore consistent with an ability of clozapine to perturb positioning and proper function of these cells, vital for T cell support of production of high affinity antibodies in response to T dependent antigens.

Further highlighting the importance of germinal centre formation to the pathogenesis of CIA is the finding that syndecan-4 null mice, which exhibit lower numbers of B cells and deficient germinal centre formation in draining lymph nodes, are resistant to CIA (Endo et al., 2015). Given the critical importance of tight regulation of germinal centres to the maintenance of self-tolerance and prevention of pathogenic autoantibody production in autoimmunity, the impact of clozapine as demonstrated in the CIA model strongly supports its potential to mitigate pathogenic autoantibody production.

### Example 5

### Study of Effect of Clozapine and Norclozapine on Human Plasma Cell Generation Using an In Vitro B Cell Differentiation System

An established in vitro platform (Cocco et al., 2012) was used to evaluate the impact of clozapine, its major metabolite norclozapine and a comparator antipsychotic drug, haloperidol, on the generation and differentiation and viability of human plasma cells.

### Method

### General:

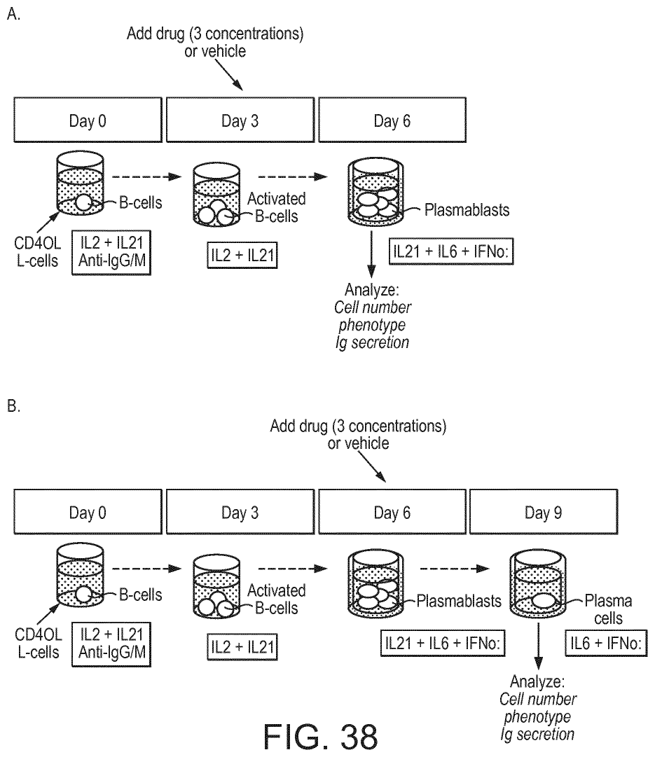

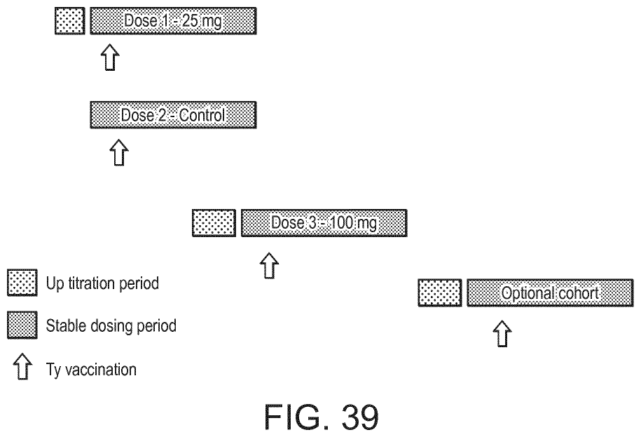

The system employed is based on a published model (Cocco et al., 2012) which uses a CD40L/IL-2/IL-21 based stimulus to drive B-cell activation and differentiation in a 3-step process to generate plasmablasts and functional polyclonal mature plasma cells (See FIG. 38), The final step of the culture (Day 6-9) was performed in the context of IFN-α driven survival signals and without stromal cells.

The experiment was performed using total peripheral blood B-cells isolated from healthy donors. The experiment was performed from four independent donors.

### Drug Addition:

Compounds were sourced from Tocris and dissolved in DMSO at the following concentrations:

### Clozapine:

350 ng/ml Clozapine (approximately equivalent to 500 mg adult human dose)
100 ng/ml Clozapine
25 ng/ml Clozapine (approximately equivalent to 55 mg adult human dose)

### Norclozapine:

200 ng/ml norclozapine
70 ng/ml norclozapine
15 ng/ml norclozapine

### Haloperidol:

25 ng/ml Haloperidol
8 ng/ml Haloperidol
2 ng/ml Haloperidol

DMSO as diluent control at 0.1%. All DMSO concentrations were adjusted to 0.1% for all drug treated samples.

Drugs were added at two time points:
day-3 of the culture (activated B-cell/pre-plasmablast), or
day-6 of the culture (plasmablast)

### Evaluation:

The cultures were evaluated 3 days after addition of the compound with day-3 drug additions evaluated at day-6 (plasmablast) and day-6 drug additions evaluated at day-9 (early plasma cell) (see FIG. 38).

### Evaluation Encompassed:

### Flow Cytometric Assessment of:

phenotype (CD19, CD20, CD27, CD38, CD138)
viability (7AAD)
cell number (bead count)

### Immunoglobulin Secretion:

ELISA analysis of total IgM/IgG from bulk supernatant collected at day 6 and day 9 of respective cultures

### Results

### Cell Phenotype:

Across all four donors the control DMSO samples demonstrated a transition to a plasmablast state from day 3 to day 6 with downregulation of CD20, upregulation of CD38 and variable upregulation of CD27 combined with retained CD19 expression and lack of CD138. On subsequent transfer into plasma cell maturation conditions the control cells showed progressive loss of CD20, downregulation of CD19 and upregulation of CD138 combined with further upregulation of CD38 and CD27 indicating transition to early plasma cell state. These findings indicate that the differentiation protocol worked in relation to phenotype and that all four samples were suitable as references for the in vitro differentiation system.

In terms of effects on phenotypic maturation none of the drugs at any concentration showed significant effects on the downregulation of the 3 cell phenotype as reflected in equivalent loss of CD20 and CD19 expression. None of the drugs at any concentration showed significant effects on the pattern of acquisition of C27 or CD138 expression at either day 6 or day 9 time points.

All three drugs showed a dose related effect on the expression of CD38 in one donor. This was modest at the day 6 time point but was significant at the day 9 time point with a substantial and reproducible shift in CD38 expression. However, this effect was not observed as a consistent effect across the other donors.

### Cell Number and Viability:

Across all four donors the control DMSO samples demonstrated an expansion to the plasmablast state from day 3 to day 6 and contraction during the transition to plasma cell state. Based on an input activated B cell number at day 3 of 10⁵ the average expansion observed during the day 3 to day 6 culture was 12-fold. There was a 5-fold contraction that accompanied the maturation to the plasma cell state from 5×10⁵ input at day 6 to 10⁵ viable cells at day 9. It was concluded that the differentiation protocol worked in relation to cell number and that all four samples are suitable as references.

None of the drugs at any concentration impacted significantly on the number of viable cells at either day 6 or day 9. This was not affected whether considering total cell number or viable cell number per input cell. Based on equivalent input activated B cell number the degree of expansion from day 3 to day 6 was equivalent across all drugs and concentrations. Equally there was no effect on the viable cell number recovered at day 9 with any drug at any concentration.

### Immunoglobulin Secretion:

Across all four donors the control DMSO samples showed evidence of significant IgM and IgG secretion at across the day 3 to day 6 culture. This was continued into the day 6 to day 9 culture with predicted higher per cell estimated secretion rates in this second culture phase to the plasma cell stated. It was concluded that the differentiation protocol worked in relation to immunoglobulin secretion and that all four samples are suitable as references.

In terms of immunoglobulin secretion there is greater variation between individual donors, but there were no clear trends in response to any of the three drugs at any dose. Normalising to DMSO as control provided the simplest view of the data and showed only minor shifts in the detected immunoglobulin in relation to IgG. Where changes are observed these follow inverse responses in relation to the dose for example norclozapine with one donor.

### Conclusion

The results showed that none of the drugs are directly toxic to differentiating B-cells, nor do any of the drugs at any concentration show consistent effects on the ability of the resulting differentiated antibody secreting cells to secrete antibody.

In terms of phenotypic responses there is variability between the donors in relation to CD38 expression with one donor in particular showing an apparent dose dependent downmodulation in the window of differentiation between plasmablast (day 6) and early plasma cell (day 9). However this response did not reproduce as a consistent feature across the other donors tested.

Overall, therefore, the compounds as tested do not show a consistent inhibitory effect on the functional or phenotypic maturation of activated B-cells to the early plasma cell state and have no effect on viability of antibody secreting cells.

The in vitro system employed has limitations in terms of being a ‘forced’ B cell differentiation assay (as opposed to physiological expansion), with a focus on peripheral B cells, limited culture duration which may not reflect effects of very chronic exposure, and lack of the normal micro-environment of B cells in primary (e.g. bone marrow) or secondary lymphoid tissues, nor indirect regulation (e.g. through T follicular helper cells and/or IL-21). Notwithstanding these, the findings suggest that clozapine is unlikely to be acting directly on plasma cells or their precursors and that the immunophenotypic findings in vivo reflect a more complex and/or indirect action. The findings from this in vitro study are consistent with the lack of reduction in overall B cell numbers (i.e. no evidence of generalized B cell depletion in patients taking clozapine),

### Summary of Results Set Out in Examples 1-5

The results set out in the examples above, encompassing observational data in humans treated with clozapine for prolonged periods of time, to short term dosing in healthy wild type mice in an immunologically unchallenged setting, to evaluation in a disease model of autoimmune disease with a major B cell component driven by antigen (CIA model), highlight several key effects of clozapine:

1. Reduction in total circulating immunoglobulin levels affecting all classes evaluated (IgG, IgM and IgA). While exhibiting interindividual variation, clozapine is seen to result in a leftward shift in the frequency distribution curve for these immunoglobulins. The robustness of this finding is highlighted by the interim findings in an orthogonal cohort of patients taking clozapine or other antipsychotics.

2. A relatively greater impact in human to reduce IgA (and IgM) compared to IgG, in part recapitulated with short-term dosing of wild type mice.

3. Evidence of progressive immunoglobulin (IgG) reduction with increasing duration of clozapine exposure in human. Conversely, evidence of gradual recovery (over years) of IgG on clozapine cessation.

4. Reduction in specific immunoglobulin. Beyond reductions in total immunoglobulin titre, clozapine is seen to lower pathogenic immunoglobulin (CIA model) and has been demonstrated by the inventors to lower pneumococcal specific antibody in human (Ponsford et al., 2018), with the latter demonstrating a strong trend to significantly lower values on even interim analysis of the second observational cohort.

5. No significant impact on overall circulating (CD19+) B cells numbers. This observation contrasts sharply with the impact of current aggressive generalised B cell depleting biological approaches.

6. Substantial reductions in circulating plasmablasts (short-lived proliferating antibody secreting cells of the B cell lineage) and class-switched memory B cells. Both cell types are critical in the immediate and secondary humoral response. Class-switching enables a B cell to switch from IgM to production of the secondary IgH isotype antibodies IgG, IgA or IgE with different effector functions (Chaudhuri and Alt, 2004). Increased class-switching and plasma cell differentiation is recognised as a key feature in autoimmune disease associated with pathogenic immunoglobulin production (Suurmond et al., 2018). An ability of clozapine to inhibit this process, i.e. reduce class-switched memory B cells, suggests particular therapeutic potential in the setting of pathogenic immunoglobulin-mediated disorders which are primarily mediated by autoantibodies of the IgG, IgA or IgE subclass.

7. Subtle effects on bone marrow B cell precursors, specifically including a reduction in total pre B cells, proliferating pre B cells and immature B cells. This is notable for being a key endogenous transition checkpoint of B cell development for autoreactivity (Melchers, 2015). Defective B cell tolerance, including early tolerance, is recognised as a fundamental feature predisposing to autoimmunity (Samuels et al., 2005; Yurasov et al., 2005). Accordingly, while speculative, it is possible that this effect of clozapine will serve to reduce further progression of B cells with autoreactivity (of the IgH chain) to modulate the emerging B cell repertoire.

8. Reduction in bone marrow long-lived plasma cells, a key cell population responsible for driving persistent autoimmune disease through the production of pathogenic immunoglobulin and which is substantially refractory to existing therapeutics.

9. The ability to substantially delay the onset of an experimental model of autoimmune disease with a substantial B cell-driven and pathogenic autoantibody component.

10. Disruption of germinal centre function through effects on its key cellular components: induction of a profound reduction in germinal centre B cells together with reduction in surface expression of key proteins regulating T follicular helper cell functionality (PD1 and CXCR5). Germinal centres are the sites of intense proliferation and somatic mutation to result in differentiation of antigen-activated B cells into high affinity memory B cells or plasma cells. Accordingly, this finding (following antigen injection in the CIA model) is consistent with an impact of clozapine on distal B cell lineage maturation/function and concordant with observations set out in the examples of reduced class switched memory B cells, reduced plasmablast and long-lived plasma cell formation. Together these actions will tend to reduce pathogenic immunoglobulin production in the setting of autoimmune disease.

11. Based on an in vitro differentiation assay, the observed effects of clozapine appear unlikely to reflect a direct effect on antibody secreting cells.

Thus, clozapine appears to have profound influence in vivo on the pathways involved in B cell maturation and pathogenic antibody (particularly pathogenic IgG and IgA antibody) production and thus is useful in treating pathogenic immunoglobulin driven B cell mediated diseases.

### Example 6

### Healthy Human Volunteer Study

This study is a randomized unblinded controlled trial investigating the effects of low-dose clozapine on B cell number and function in healthy volunteers following vaccination (i.e. antigenic challenge). The study employs a parallel arm design (see FIG. 39) with a delayed start for the higher dose tested. In this study a total of up to 48 healthy volunteers will be recruited in to up to 4 cohorts. All participants will be administered Typhi immunization to stimulate the production of specific immunoglobulin (specifically IgG) at day 1 (immunization day) and followed for a period of approximately 56 days. Cohort 1 (n=12 participants) will be administered 25 mg of clozapine for 28 days and followed up for a further 28 days, whilst cohort 2 (n=12 participants, which will be recruited in parallel with Cohort1) will not receive any clozapine but will undergo vaccination. Cohort 2 will be followed in the same manner as cohort 1. Cohort 3 (100 mg clozapine) will only be initiated after the data from the active clozapine treatment period in cohort 1 (day 28 of active treatment) is reviewed by a Safety Committee. There is the potential for an optional cohort of another 12 healthy volunteers to be started if the data warrants further evaluation of doses between 25 and 100 mg clozapine.

Participants in Cohorts 1 and 2 will remain in the trial for a total of 60 days excluding their initial screening visit. Participants in Cohort 3 will take part for a total of 70 days excluding their initial screening visit.

The duration of participation for participants in the optional cohort 4 will vary depending on the dose chosen, due to the titration period being altered accordingly, but excluding their initial screening visit participants will participate for a maximum of 63 days (if a 100 mg dose is selected).

### Objectives and Outcome Measures

| | | Time point(s) of |
|---|---|---|
| | | evaluation of this |
| | | outcome measure |
| Objectives | Outcome Measures | (if applicable) |
| Primary Objective | Difference in specific anti-Typhim Vi | 28 days after |
| To understand the effect of | IgG 28 days after vaccination | vaccination |
| clozapine on primary | | |
| vaccination response | | |
| Secondary Objectives | Change from baseline in total | 28 days after |
| To determine the effect of | immunoglobulin levels (IgG, | vaccination |
| clozapine on circulating | IgM and IgA subclasses) | |
| immunoglobulin levels | | |
| To determine the effect of | Plasmablast response at seven | 7 days after |
| clozapine on circulating | days post- vaccination | vaccination |
| plasmablast levels | | |
| Exploratory Objectives | | |
| To understand the exposure- | Concentration response analysis to | All available |
| response relationship of clozapine | each primary and secondary end point | timepoints |
| on B cell subsets and | | |
| immunoglobulins | | |
| Effect of clozapine on | The difference in changes of specific | 28 days after |
| transcription profiles of sorted | RNA expression pre-clozapine dosing | vaccination |
| immune cells pre- and post- | and 28 days after vaccination | |
| therapy | between clozapine and control cohorts | |

Similar Immune Biomarkers will be collected in the Healthy Volunteer study to those in the observational study (Example 2).

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

### REFERENCES

Adam, L., Zoldan, K., Hofmann, M., Schultheiss, M., Bettinger, D., Neumann-Haefelin, C., Thimme, R., and Boettler, T. (2018). Follicular T Helper Cell Signatures in Primary Biliary Cholangitis and Primary Sclerosing Cholangitis. Hepatology communications 2, 1051-1063.
Akashi, T., Nagafuchi, S., Anzai, K., Kondo, S., Kitamura, D., Wakana, S., Ono, J., Kikuchi, M., Niho, Y., and Watanabe, T. (1997). Direct evidence for the contribution of B cells to the progression of insulitis and the development of diabetes in non-obese diabetic mice. International immunology 9, 1159-1164.
Albers, L. N., Zone, J. J., Stoff, B. K., and Feldman, R. J. (2017). Rituximab Treatment for Recalcitrant Dermatitis Herpetiformis. JAMA dermatology 153, 315-318.
Alsaleh, G., Francois, A., Knapp, A. M., Schickel, J. N., Sibilia, J., Pasquali, J. L., Gottenberg, J. E., Wachsmann, D., and Soulas-Sprauel, P. (2011). Synovial fibroblasts promote immunoglobulin class switching by a mechanism involving BAFF. European journal of immunology 41, 2113-2122.
Anhalt, G. J., Labib, R. S., Voorhees, J. J., Beals, T. F., and Diaz, L. A. (1982). Induction of pemphigus in neonatal mice by passive transfer of IgG from patients with the disease. The New England journal of medicine 306, 1189-1196.
Arnold, D. M., Vrbensky, J. R., Karim, N., Smith, J. W., Liu, Y., Ivetic, N., Kelton, J. G., and Nazy, I. (2017). The effect of rituximab on anti-platelet autoantibody levels in patients with immune thrombocytopenia. British journal of haematology 178, 302-307.
Asashima, N., Fujimoto, M., Watanabe, R., Nakashima, H., Yazawa, N., Okochi, H., and Tamaki, K. (2006). Serum levels of BAFF are increased in bullous pemphigoid but not in Pemphigus vulgaris. The British journal of dermatology 155, 330-336.
Audia, S., Rossato, M., Santegoets, K., Spijkers, S., Wichers, C., Bekker, C., Bloem, A., Boon, L., Flinsenberg, T., Compeer, E., et al. (2014). Splenic TFH expansion participates in B-cell differentiation and antiplatelet-antibody production during immune thrombocytopenia. Blood 124, 2858-2866.
Audia, S., Samson, M., Guy, J., Janikashvili, N., Fraszczak, J., Trad, M., Ciudad, M., Leguy, V., Berthier, S., Petrella, T., et al. (2011). Immunologic effects of rituximab on the human spleen in immune thrombocytopenia. Blood 118, 4394-4400.
Baker, D., Marta, M., Pryce, G., Giovannoni, G., and Schmierer, K. (2017). Memory B Cells are Major Targets for Effective Immunotherapy in Relapsing Multiple Sclerosis. EBioMedicine 16, 41-50.
Banchereau, R., Hong, S., Cantarel, B., Baldwin, N., Baisch, J., Edens, M., Cepika, A. M., Acs, P., Turner, J., Anguiano, E., et al. (2016). Personalized Immunomonitoring Uncovers Molecular Networks that Stratify Lupus Patients. Cell 165, 551-565.
Barcellini, W. (2015). New Insights in the Pathogenesis of Autoimmune Hemolytic Anemia. Transfusion medicine and hemotherapy: offizielles Organ der Deutschen Gesellschaft fur Transfusionsmedizin and Immunhamatologie 42, 287-293.
Bedeir, A. S., Lash, R. H., Lash, J. G., and Ray, M. B. (2010). Significant increase in IgG4+ plasma cells in gastric biopsy specimens from patients with pernicious anaemia. Journal of clinical pathology 63, 999-1001.
Beland, K., Marceau, G., Labardy, A., Bourbonnais, S., and Alvarez, F. (2015). Depletion of B cells induces remission of autoimmune hepatitis in mice through reduced antigen presentation and help to T cells. Hepatology (Baltimore, Md.) 62, 1511-1523.
Bello, S., Menendez, R., Antoni, T., Reyes, S., Zalacain, R., Capelastegui, A., Aspa, J., Borderias, L., Martin-Villasclaras, J. J., Alfageme, I., et al. (2014). Tobacco smoking increases the risk for death from pneumococcal pneumonia. Chest 146, 1029-1037.
Belnoue, E., Pihlgren, M., McGaha, T. L., Tougne, C., Rochat, A. F., Bossen, C., Schneider, P., Huard, B., Lambert, P. H., and Siegrist, C. A. (2008). APRIL is critical for plasmablast survival in the bone marrow and poorly expressed by early-life bone marrow stromal cells. Blood 111, 2755-2764.
Ben-Skowronek, I., Szewczyk, L., Kulik-Rechberger, B., and Korobowicz, E. (2013). The differences in T and B cell subsets in thyroid of children with Graves' disease and Hashimoto's thyroiditis. World journal of pediatrics: WJP 9, 245-250.
Benner, R., Hijmans, W., and Haaijman, J. J. (1981). The bone marrow: the major source of serum immunoglobulins, but still a neglected site of antibody formation. Clinical and experimental immunology 46, 1-8.
Bennett, J. L., O'Connor, K. C., Bar-Or, A., Zamvil, S. S., Hemmer, B., Tedder, T. F., von Budingen, H. C., Stuve, O., Yeaman, M. R., Smith, T. J., et al. (2015). B lymphocytes in neuromyelitis optica. Neurology(R) neuroimmunology & neuroinflammation 2, e104.
Berglin, L., Bjorkstrom, N. K., and Bergquist, A. (2013). Primary sclerosing cholangitis is associated with autoreactive IgA antibodies against biliary epithelial cells. Scandinavian journal of gastroenterology 48, 719-728.
Bergqvist, P., Stensson, A., Lycke, N. Y., and Bemark, M. (2010). T cell-independent IgA class switch recombination is restricted to the GALT and occurs prior to manifest germinal center formation. Journal of immunology (Baltimore, Md.: 1950) 184, 3545-3553.
Bermejo, D. A., Jackson, S. W., Gorosito-Serran, M., Acosta-Rodriguez, E. V., Amezcua-Vesely, M. C., Sather, B. D., Singh, A. K., Khim, S., Mucci, J., Liggitt, D., et al. (2013). Trypanosoma cruzi trans-sialidase initiates a program independent of the transcription factors RORgammat and Ahr that leads to IL-17 production by activated B cells. Nature immunology 14, 514-522.
Berthoux, F., Suzuki, H., Thibaudin, L., Yanagawa, H., Maillard, N., Mariat, C., Tomino, Y., Julian, B. A., and Novak, J. (2012). Autoantibodies targeting galactose-deficient IgA1 associate with progression of IgA nephropathy. Journal of the American Society of Nephrology: JASN 23, 1579-1587.
Bessis, N., Decker, P., Assier, E., Semerano, L., and Boissier, M. C. (2017). Arthritis models: usefulness and interpretation. Seminars in immunopathology 39, 469-486.
Betterle, C., Volpato, M., Rees Smith, B., Furmaniak, J., Chen, S., Greggio, N. A., Sanzari, M., Tedesco, F., Pedini, B., Boscaro, M., et al. (1997). I. Adrenal cortex and steroid 21-hydroxylase autoantibodies in adult patients with organ-specific autoimmune diseases: markers of low progression to clinical Addison's disease. The Journal of clinical endocrinology and metabolism 82, 932-938.
Bien, C. G., Vincent, A., Barnett, M. H., Becker, A. J., Blumcke, I., Graus, F., Jellinger, K. A., Reuss, D. E., Ribalta, T., Schlegel, J., et al. (2012). Immunopathology of autoantibody-associated encephalitides: clues for pathogenesis. Brain: a journal of neurology 135, 1622-1638.
Birgens, H., Frederiksen, H., Hasselbalch, H. C., Rasmussen, I. H., Nielsen, O. J., Kjeldsen, L., Larsen, H., Mourits-Andersen, T., Plesner, T., Ronnov-Jessen, D., et al. (2013). A phase III randomized trial comparing glucocorticoid monotherapy versus glucocorticoid and rituximab in patients with autoimmune haemolytic anaemia. British journal of haematology 163, 393-399.
Bizzaro, N., and Antico, A. (2014). Diagnosis and classification of pernicious anemia. Autoimmunity reviews 13, 565-568.
Bloise, F. F., Oliveira, F. L., Nobrega, A. F., Vasconcellos, R., Cordeiro, A., Paiva, L. S., Taub, D. D., Borojevic, R., Pazos-Moura, C. C., and Mello-Coelho, V. (2014). High levels of circulating triiodothyronine induce plasma cell differentiation. The Journal of endocrinology 220, 305-317.
Brand, D. D., Kang, A. H., and Rosloniec, E. F. (2004). The mouse model of collagen-induced arthritis. Methods in molecular medicine 102, 295-312.
Brand, D. D., Latham, K. A., and Rosloniec, E. F. (2007). Collagen-induced arthritis. Nature protocols 2, 1269-1275.
Bratland, E., and Husebye, E. S. (2011). Cellular immunity and immunopathology in autoimmune Addison's disease. Molecular and cellular endocrinology 336, 180-190.
Brenner, T., Sills, G. J., Hart, Y., Howell, S., Waters, P., Brodie, M. J., Vincent, A., and Lang, B. (2013). Prevalence of neurologic autoantibodies in cohorts of patients with new and established epilepsy. Epilepsia 54, 1028-1035.
Britton, J. (2016). Autoimmune epilepsy. Handbook of clinical neurology 133, 219-245.
Bryant, V. L., Ma, C. S., Avery, D. T., Li, Y., Good, K. L., Corcoran, L. M., de Waal Malefyt, R., and Tangye, S. G. (2007). Cytokine-mediated regulation of human B cell differentiation into Ig-secreting cells: predominant role of IL-21 produced by CXCR5+ T follicular helper cells. Journal of immunology (Baltimore, Md.: 1950) 179, 8180-8190.
Budeus, B., Schweigle de Reynoso, S., Przekopowitz, M., Hoffmann, D., Seifert, M., and Kuppers, R. (2015). Complexity of the human memory B-cell compartment is determined by the versatility of clonal diversification in germinal centers. Proceedings of the National Academy of Sciences of the United States of America 112, E5281-5289.
Burak, K. W., Swain, M. G., Santodomingo-Garzon, T., Lee, S. S., Urbanski, S. J., Aspinall, A. I., Coffin, C. S., and Myers, R. P. (2013). Rituximab for the treatment of patients with autoimmune hepatitis who are refractory or intolerant to standard therapy. Canadian journal of gastroenterology=Journal canadien de gastroenterologie 27, 273-280.
Burman, P., Kampe, O., Kraaz, W., Loof, L., Smolka, A., Karlsson, A., and Karlsson-Parra, A. (1992). A study of autoimmune gastritis in the postpartum period and at a 5-year follow-up. Gastroenterology 103, 934-942.
Burton, P. R., Clayton, D. G., Cardon, L. R., Craddock, N., Deloukas, P., Duncanson, A., Kwiatkowski, D. P., McCarthy, M. I., Ouwehand, W. H., Samani, N. J., et al. (2007). Association scan of 14,500 nonsynonymous SNPs in four diseases identifies autoimmunity variants. Nature genetics 39, 1329-1337.
Caja, S., Maki, M., Kaukinen, K., and Lindfors, K. (2011). Antibodies in celiac disease: implications beyond diagnostics. Cellular & molecular immunology 8, 103-109.
Caproni, M., Volpi, W., Giomi, B., Cardinali, C., Antiga, E., Melani, L., Dagata, A., and Fabbri, P. (2004). Chronic idiopathic and chronic autoimmune urticaria: clinical and immunopathological features of 68 subjects. Acta dermato-venereologica 84, 288-290.
Care, M. A., Stephenson, S. J., Barnes, N. A., Fan, I., Zougman, A., El-Sherbiny, Y. M., Vital, E. M., Westhead, D. R., Tooze, R. M., and Doody, G. M. (2016). Network Analysis Identifies Proinflammatory
Plasma Cell Polarization for Secretion of ISG15 in Human Autoimmunity. Journal of immunology (Baltimore, Md.: 1950) 197, 1447-1459.
Carey, E. J., Ali, A. H., and Lindor, K. D. (2015). Primary biliary cirrhosis. Lancet (London, England) 386, 1565-1575.
Casola, S., Otipoby, K. L., Alimzhanov, M., Humme, S., Uyttersprot, N., Kutok, J. L., Carroll, M. C., and Rajewsky, K. (2004). B cell receptor signal strength determines B cell fate. Nature immunology 5, 317-327.
Cepok, S., Rosche, B., Grummel, V., Vogel, F., Zhou, D., Sayn, J., Sommer, N., Hartung, H. P., and Hemmer, B. (2005). Short-lived plasma blasts are the main B cell effector subset during the course of multiple sclerosis. Brain: a journal of neurology 128, 1667-1676.
Cervenak, L., Magyar, A., Boja, R., and Laszlo, G. (2001). Differential expression of GL7 activation antigen on bone marrow B cell subpopulations and peripheral B cells. Immunology letters 78, 89-96.
Chakravarty, S. D., Yee, A. F., and Paget, S. A. (2011). Rituximab successfully treats refractory chronic autoimmune urticaria caused by IgE receptor autoantibodies. The Journal of allergy and clinical immunology 128, 1354-1355.
Chaudhuri, J., and Alt, F. W. (2004). Class-switch recombination: interplay of transcription, DNA deamination and DNA repair. Nature reviews Immunology 4, 541-552.
Chavele, K. M., Merry, E., and Ehrenstein, M. R. (2015). Cutting edge: circulating plasmablasts induce the differentiation of human T follicular helper cells via IL-6 production. Journal of immunology (Baltimore, Md.: 1950) 194, 2482-2485.
Chen, X., Ma, W., Zhang, T., Wu, L., and Qi, H. (2015). Phenotypic Tfh development promoted by CXCR5-controlled re-localization and IL-6 from radiation-resistant cells. Protein & cell 6, 825-832.
Chihara, N., Aranami, T., Oki, S., Matsuoka, T., Nakamura, M., Kishida, H., Yokoyama, K., Kuroiwa, Y., Hattori, N., Okamoto, T., et al. (2013). Plasmablasts as migratory IgG-producing cells in the pathogenesis of neuromyelitis optica. PloS one 8, e83036.
Chihara, N., Aranami, T., Sato, W., Miyazaki, Y., Miyake, S., Okamoto, T., Ogawa, M., Toda, T., and Yamamura, T. (2011). Interleukin 6 signaling promotes anti-aquaporin 4 autoantibody production from plasmablasts in neuromyelitis optica. Proceedings of the National Academy of Sciences of the United States of America 108, 3701-3706.
Chu, V. T., and Berek, C. (2013). The establishment of the plasma cell survival niche in the bone marrow. Immunological reviews 251, 177-188.
Chu, V. T., Frohlich, A., Steinhauser, G., Scheel, T., Roch, T., Fillatreau, S., Lee, J. J., Lohning, M., and Berek, C. (2011a). Eosinophils are required for the maintenance of plasma cells in the bone marrow. Nature immunology 12, 151-159.
Chu, X., Pan, C. M., Zhao, S. X., Liang, J., Gao, G. Q., Zhang, X. M., Yuan, G. Y., Li, C. G., Xue, L. Q., Shen, M., et al. (2011b). A genome-wide association study identifies two new risk loci for Graves' disease. Nature genetics 43, 897-901.
Chua, I., Lagos, M., Charalambous, B. M., Workman, S., Chee, R., and Grimbacher, B. (2011). Pathogen-specific IgG antibody levels in immunodeficient patients receiving immunoglobulin replacement do not provide additional benefit to therapeutic management over total serum IgG. The Journal of allergy and clinical immunology 127, 1410-1411.
Chung, B. K., Guevel, B. T., Reynolds, G. M., Gupta Udatha, D. B., Henriksen, E. K., Stamataki, Z., Hirschfield, G. M., Karlsen, T. H., and Liaskou, E. (2017). Phenotyping and auto-antibody production by liver-infiltrating B cells in primary sclerosing cholangitis and primary biliary cholangitis. Journal of autoimmunity 77, 45-54.
Chung, B. K., Henriksen, E. K. K., Jorgensen, K. K., Karlsen, T. H., Hirschfield, G. M., and Liaskou, E. (2018). Gut and Liver B Cells of Common Clonal Origin in Primary Sclerosing Cholangitis-Inflammatory Bowel Disease. Hepatology communications 2, 956-967.
Chung, B. K., Udatha, G., Pharo, A., Taussig, M., Fonseca, C., Stoevesandt, O., Mehta, A., Lund-Johansen, F., Hirschfield, G. M., Karlsen, T. H., et al. (2016). Liver-Infiltrating B Cells Produce Autoantibodies that Point toward Novel Autoantigens in Primary Sclerosing Cholangitis. Journal of hepatology 64, S433.
Cocco, M., Stephenson, S., Care, M. A., Newton, D., Barnes, N. A., Davison, A., Rawstron, A., Westhead, D. R., Doody, G. M., and Tooze, R. M. (2012). In vitro generation of long-lived human plasma cells. Journal of immunology (Baltimore, Md.: 1950) 189, 5773-5785.
Collin, P., Salmi, T. T., Hervonen, K., Kaukinen, K., and Reunala, T. (2017). Dermatitis herpetiformis: a cutaneous manifestation of coeliac disease. Annals of medicine 49, 23-31.
Colliou, N., Picard, D., Caillot, F., Calbo, S., Le Corre, S., Lim, A., Lemercier, B., Le Mauff, B., Maho-Vaillant, M., Jacquot, S., et al. (2013). Long-term remissions of severe pemphigus after rituximab therapy are associated with prolonged failure of desmoglein B cell response. Science translational medicine 5, 175ra130.
Colucci, R., Lotti, F., Dragoni, F., Arunachalam, M., Lotti, T., Benvenga, S., and Moretti, S. (2014). High prevalence of circulating autoantibodies against thyroid hormones in vitiligo and correlation with clinical and historical parameters of patients. The British journal of dermatology 171, 786-798.
Combalia, A., Losno, R. A., Prieto-Gonzalez, S., and Mascaro, J. M. (2018). Rituximab in Refractory Chronic Spontaneous Urticaria: An Encouraging Therapeutic Approach. Skin pharmacology and physiology 31, 184-187.
Craig, S. W., and Cebra, J. J. (1971). Peyer's patches: an enriched source of precursors for IgA-producing immunocytes in the rabbit. The Journal of experimental medicine 134, 188-200.
Cross, A. H., Stark, J. L., Lauber, J., Ramsbottom, M. J., and Lyons, J. A. (2006). Rituximab reduces B cells and T cells in cerebrospinal fluid of multiple sclerosis patients. Journal of neuroimmunology 180, 63-70.
Cui, J., Arita, Y., and Bystryn, J. C. (1993). Cytolytic antibodies to melanocytes in vitiligo. The Journal of investigative dermatology 100, 812-815.
Cui, J., Harning, R., Henn, M., and Bystryn, J. C. (1992). Identification of pigment cell antigens defined by vitiligo antibodies. The Journal of investigative dermatology 98, 162-165.
Dandah, A., Habir, K., Nandakumar, K. S., Saxena, A., Xu, B., Holmdahl, R., and Malin, S. (2018). Germinal Center B Cells Are Essential for Collagen-Induced Arthritis. Arthritis & rheumatology (Hoboken, N.J.) 70, 193-203.
Dalin, F., Nordling Eriksson, G., Dahlqvist, P., Hallgren, A., Wahlberg, J., Ekwall, O., Soderberg, S., Ronnelid, J., Olcen, P., Winqvist, O., et al. (2017). Clinical and Immunological Characteristics of Autoimmune Addison Disease: A Nationwide Swedish Multicenter Study. The Journal of clinical endocrinology and metabolism 102, 379-389.
Dalmau, J., Gleichman, A. J., Hughes, E. G., Rossi, J. E., Peng, X., Lai, M., Dessain, S. K., Rosenfeld, M. R., Balice-Gordon, R., and Lynch, D. R. (2008). Anti-NMDA-receptor encephalitis: case series and analysis of the effects of antibodies. The Lancet Neurology 7, 1091-1098.
Damato, V., Evoli, A., and Iorio, R. (2016). Efficacy and Safety of Rituximab Therapy in Neuromyelitis Optica Spectrum Disorders: A Systematic Review and Meta-analysis. JAMA neurology 73, 1342-1348.
De Bellis, A. A., Falorni, A., Laureti, S., Perrino, S., Coronella, C., Forini, F., Bizzarro, E., Bizzarro, A., Abbate, G., and Bellastella, A. (2001). Time course of 21-hydroxylase antibodies and long-term remission of subclinical autoimmune adrenalitis after corticosteroid therapy: case report. The Journal of clinical endocrinology and metabolism 86, 675-678.
de Montjoye, L., Herman, A., Nicolas, J. F., and Baeck, M. (2018). Treatment of chronic spontaneous urticaria: Immunomodulatory approaches. Clinical immunology (Orlando, Fla.) 190, 53-63.
De Vita, S., Quartuccio, L., Isola, M., Mazzaro, C., Scaini, P., Lenzi, M., Campanini, M., Naclerio, C., Tavoni, A., Pietrogrande, M., et al. (2012). A randomized controlled trial of rituximab for the treatment of severe cryoglobulinemic vasculitis. Arthritis and rheumatism 64, 843-853.
Di Zenzo, G., Di Lullo, G., Corti, D., Calabresi, V., Sinistro, A., Vanzetta, F., Didona, B., Cianchini, G., Hertl, M., Eming, R., et al. (2012). Pemphigus autoantibodies generated through somatic mutations target the desmoglein-3 cis-interface. The Journal of clinical investigation 122, 3781-3790.
Diaz, L. A., Ratrie, H., 3rd, Saunders, W. S., Futamura, S., Squiquera, H. L., Anhalt, G. J., and Giudice, G. J. (1990). Isolation of a human epidermal cDNA corresponding to the 180-kD autoantigen recognized by bullous pemphigoid and herpes gestationis sera. Immunolocalization of this protein to the hemidesmosome. The Journal of clinical investigation 86, 1088-1094.
Dieterich, W., Ehnis, T., Bauer, M., Donner, P., Volta, U., Riecken, E. O., and Schuppan, D. (1997). Identification of tissue transglutaminase as the autoantigen of celiac disease. Nature medicine 3, 797-801.
Dogan Onugoren, M., Golombeck, K. S., Bien, C., Abu-Tair, M., Brand, M., Bulla-Hellwig, M., Lohmann, H., Munstermann, D., Pavenstadt, H., Tholking, G., et al. (2016). Immunoadsorption therapy in autoimmune encephalitides. Neurology(R) neuroimmunology & neuroinflammation 3, e207.
Drachman, D. B., Adams, R. N., Josifek, L. F., and Self, S. G. (1982). Functional activities of autoantibodies to acetylcholine receptors and the clinical severity of myasthenia gravis. The New England journal of medicine 307, 769-775.
Drachman, D. B., Angus, C. W., Adams, R. N., Michelson, J. D., and Hoffman, G. J. (1978). Myasthenic antibodies cross-link acetylcholine receptors to accelerate degradation. The New England journal of medicine 298, 1116-1122.
du Pre, M. F., and Sollid, L. M. (2015). T-cell and B-cell immunity in celiac disease. Best practice & research Clinical gastroenterology 29, 413-423.
Eggers, E. L., Michel, B. A., Wu, H., Wang, S. Z., Bevan, C. J., Abounasr, A., Pierson, N. S., Bischof, A., Kazer, M., Leitner, E., et al. (2017). Clonal relationships of CSF B cells in treatment-naive multiple sclerosis patients. JCI insight 2.
Elliott, C., Lindner, M., Arthur, A., Brennan, K., Jarius, S., Hussey, J., Chan, A., Stroet, A., Olsson, T., Willison, H., et al. (2012). Functional identification of pathogenic autoantibody responses in patients with multiple sclerosis. Brain: a journal of neurology 135, 1819-1833.
Elston, D. M., McCollough, M. L., Bergfeld, W. F., Liranzo, M. O., and Heibel, M. (1997). Eosinophils in fibrous tracts and near hair bulbs: a helpful diagnostic feature of alopecia areata. Journal of the American Academy of Dermatology 37, 101-106.
Endo, T., Ito, K., Morimoto, J., Kanayama, M., Ota, D., Ikesue, M., Kon, S., Takahashi, D., Onodera, T., Iwasaki, N., et al. (2015). Syndecan 4 Regulation of the Development of Autoimmune Arthritis in Mice by Modulating B Cell Migration and Germinal Center Formation. Arthritis & rheumatology (Hoboken, N.J.) 67, 2512-2522.
Engel, A. G., and Arahata, K. (1987). The membrane attack complex of complement at the endplate in myasthenia gravis. Annals of the New York Academy of Sciences 505, 326-332.
Eriksson, D., Bianchi, M., Landegren, N., Nordin, J., Dalin, F., Mathioudaki, A., Eriksson, G. N., Hultin-Rosenberg, L., Dahlqvist, J., Zetterqvist, H., et al. (2016). Extended exome sequencing identifies BACH2 as a novel major risk locus for Addison's disease. Journal of internal medicine 280, 595-608.
Ezzedine, K., Eleftheriadou, V., Whitton, M., and van Geel, N. (2015). Vitiligo. Lancet (London, England) 386, 74-84.
Fagiolo, E. (2004). Immunological tolerance loss vs. erythrocyte self antigens and cytokine network disregulation in autoimmune hemolytic anaemia. Autoimmunity reviews 3, 53-59.
Farooqi, M. S., Lai, Y., Lancaster, E., Schmitt, S. E., and Sachais, B. S. (2015). Therapeutic plasma exchange and immunosuppressive therapy in a patient with anti-GAD antibody-related epilepsy: quantification of the antibody response. Journal of clinical apheresis 30, 8-14.
Fiebiger, E., Maurer, D., Holub, H., Reininger, B., Hartmann, G., Woisetschlager, M., Kinet, J. P., and Stingl, G. (1995). Serum IgG autoantibodies directed against the alpha chain of Fc epsilon RI: a selective marker and pathogenetic factor for a distinct subset of chronic urticaria patients? The Journal of clinical investigation 96, 2606-2612.
Firkin, B. G., Wright, R., Miller, S., and Stokes, E. (1969). Splenic macrophages in thrombocytopenia. Blood 33, 240-245.
Fritz, J. H., Rojas, O. L., Simard, N., McCarthy, D. D., Hapfelmeier, S., Rubino, S., Robertson, S. J., Larijani, M., Gosselin, J., Ivanov, II, et al. (2011). Acquisition of a multifunctional IgA+ plasma cell phenotype in the gut. Nature 481, 199-203.
Fujii, Y., Monden, Y., Hashimoto, J., Nakahara, K., and Kawashima, Y. (1985a). Acetylcholine receptor antibody-producing cells in thymus and lymph nodes in myasthenia gravis. Clinical immunology and immunopathology 34, 141-146.
Fujii, Y., Monden, Y., Hashimoto, J., Nakahara, K., and Kawashima, Y. (1985b). Acetylcholine receptor antibody production by bone marrow cells in a patient with myasthenia gravis. Neurology 35, 577-579.
Fujiwara, K., Fukuda, Y., and Yokosuka, O. (2008). Precise histological evaluation of liver biopsy specimen is indispensable for diagnosis and treatment of acute-onset autoimmune hepatitis. Journal of gastroenterology 43, 951-958.
Futei, Y., Amagai, M., Ishii, K., Kuroda-Kinoshita, K., Ohya, K., and Nishikawa, T. (2001). Predominant IgG4 subclass in autoantibodies of Pemphigus vulgaris and foliaceus. Journal of dermatological science 26, 55-61.
Ganor, Y., Goldberg-Stern, H., Lerman-Sagie, T., Teichberg, V. I., and Levite, M. (2005). Autoimmune epilepsy: distinct subpopulations of epilepsy patients harbor serum autoantibodies to either glutamate/AMPA receptor GluR3, glutamate/NMDA receptor subunit NR2A or double-stranded DNA. Epilepsy research 65, 11-22.
Garvey, B. (2008). Rituximab in the treatment of autoimmune haematological disorders. British journal of haematology 141, 149-169.
Gottumukkala, R. V., Gavalas, N. G., Akhtar, S., Metcalfe, R. A., Gawkrodger, D. J., Haycock, J. W., Watson, P. F., Weetman, A. P., and Kemp, E. H. (2006). Function-blocking autoantibodies to the melanin-concentrating hormone receptor in vitiligo patients. Laboratory investigation; a journal of technical methods and pathology 86, 781-789.
Grattan, C. E., Francis, D. M., Hide, M., and Greaves, M. W. (1991). Detection of circulating histamine releasing autoantibodies with functional properties of anti-IgE in chronic urticaria. Clinical and experimental allergy: journal of the British Society for Allergy and Clinical Immunology 21, 695-704.
Grattan, C. E., Francis, D. M., Slater, N. G., Barlow, R. J., and Greaves, M. W. (1992). Plasmapheresis for severe, unremitting, chronic urticaria. Lancet (London, England) 339, 1078-1080.
Gray, L., van den Buuse, M., Scarr, E., Dean, B., and Hannan, A. J. (2009). Clozapine reverses schizophrenia-related behaviours in the metabotropic glutamate receptor 5 knockout mouse: association with N-methyl-D-aspartic acid receptor up-regulation. The international journal of neuropsychopharmacology 12, 45-60.
Greeley, S. A., Katsumata, M., Yu, L., Eisenbarth, G. S., Moore, D. J., Goodarzi, H., Barker, C. F., Naji, A., and Noorchashm, H. (2002). Elimination of maternally transmitted autoantibodies prevents diabetes in nonobese diabetic mice. Nature medicine 8, 399-402.
Gresa-Arribas, N., Titulaer, M. J., Torrents, A., Aguilar, E., McCracken, L., Leypoldt, F., Gleichman, A. J., Balice-Gordon, R., Rosenfeld, M. R., Lynch, D., et al. (2014). Antibody titres at diagnosis and during follow-up of anti-NMDA receptor encephalitis: a retrospective study. The Lancet Neurology 13, 167-177.
Guitton, C., Kinowski, J. M., Abbar, M., Chabrand, P., and Bressolle, F. (1999). Clozapine and metabolite concentrations during treatment of patients with chronic schizophrenia. Journal of clinical pharmacology 39, 721-728.
Guo, H., Cheng, Y., Shapiro, J., and McElwee, K. (2015). The role of lymphocytes in the development and treatment of alopecia areata. Expert review of clinical immunology 11, 1335-1351.
Halstensen, T. S., Hvatum, M., Scott, H., Fausa, O., and Brandtzaeg, P. (1992). Association of subepithelial deposition of activated complement and immunoglobulin G and M response to gluten in celiac disease. Gastroenterology 102, 751-759.
Halttunen, T., and Maki, M. (1999). Serum immunoglobulin A from patients with celiac disease inhibits human T84 intestinal crypt epithelial cell differentiation. Gastroenterology 116, 566-572.
Hammarlund, E., Thomas, A., Amanna, I. J., Holden, L. A., Slayden, O. D., Park, B., Gao, L., and Slifka, M. K. (2017). Plasma cell survival in the absence of B cell memory. Nature communications 8, 1781.
Han, S. S., Yang, S. H., Choi, M., Kim, H. R., Kim, K., Lee, S., Moon, K. C., Kim, J. Y., Lee, H., Lee, J. P., et al. (2016). The Role of TNF Superfamily Member 13 in the Progression of IgA Nephropathy. Journal of the American Society of Nephrology: JASN 27, 3430-3439.
Handin, R. I., and Stossel, T. P. (1974). Phagocytosis of antibody-coated platelets by human granulocytes. The New England journal of medicine 290, 989-993.
Harper, S. J., Allen, A. C., Layward, L., Hattersley, J., Veitch, P. S., and Feehally, J. (1994). Increased immunoglobulin A and immunoglobulin A1 cells in bone marrow trephine biopsy specimens in immunoglobulin A nephropathy. American journal of kidney diseases: the official journal of the National Kidney Foundation 24, 888-892.
Harrington, W. J., Minnich, V., Hollingsworth, J. W., and Moore, C. V. (1951). Demonstration of a thrombocytopenic factor in the blood of patients with thrombocytopenic purpura. The Journal of laboratory and clinical medicine 38, 1-10.
Hauser, S. L., Bar-Or, A., Comi, G., Giovannoni, G., Hartung, H. P., Hemmer, B., Lublin, F., Montalban, X., Rammohan, K. W., Selmaj, K., et al. (2017). Ocrelizumab versus Interferon Beta-1a in Relapsing Multiple Sclerosis. The New England journal of medicine 376, 221-234.
Hauser, S. L., Waubant, E., Arnold, D. L., Vollmer, T., Antel, J., Fox, R. J., Bar-Or, A., Panzara, M., Sarkar, N., Agarwal, S., et al. (2008). B-cell depletion with rituximab in relapsing-remitting multiple sclerosis. The New England journal of medicine 358, 676-688.
He, R., Reid, D. M., Jones, C. E., and Shulman, N. R. (1994). Spectrum of Ig classes, specificities, and titers of serum antiglycoproteins in chronic idiopathic thrombocytopenic purpura. Blood 83, 1024-1032.
He, Y., Shimoda, M., Ono, Y., Villalobos, I. B., Mitra, A., Konia, T., Grando, S. A., Zone, J. J., and Maverakis, E. (2015). Persistence of Autoreactive IgA-Secreting B Cells Despite Multiple Immunosuppressive Medications Including Rituximab. JAMA dermatology 151, 646-650.
Hegazy, S., Bouchouicha, S., Khaled, A., Laadher, L., Sellami, M. K., and Zeglaoui, F. (2016). IgA pemphigus showing IgA antibodies to desmoglein 1 and 3. Dermatology practical & conceptual 6, 31-33.
Hide, M., Francis, D. M., Grattan, C. E., Hakimi, J., Kochan, J. P., and Greaves, M. W. (1993). Autoantibodies against the high-affinity IgE receptor as a cause of histamine release in chronic urticaria. The New England journal of medicine 328, 1599-1604.
Hiepe, F., Dorner, T., Hauser, A. E., Hoyer, B. F., Mei, H., and Radbruch, A. (2011). Long-lived autoreactive plasma cells drive persistent autoimmune inflammation. Nature reviews Rheumatology 7, 170-178.
Hinze-Selch, D., Becker, E. W., Stein, G. M., Berg, P. A., Mullington, J., Holsboer, F., and Pollmacher, T. (1998). Effects of clozapine on in vitro immune parameters: a longitudinal study in clozapine-treated schizophrenic patients. Neuropsychopharmacology: official publication of the American College of Neuropsychopharmacology 19, 114-122.
Hov, J. R., Boberg, K. M., and Karlsen, T. H. (2008). Autoantibodies in primary sclerosing cholangitis. World journal of gastroenterology 14, 3781-3791.
Hoyer, B. F., Moser, K., Hauser, A. E., Peddinghaus, A., Voigt, C., Eilat, D., Radbruch, A., Hiepe, F., and Manz, R. A. (2004). Short-lived plasmablasts and long-lived plasma cells contribute to chronic humoral autoimmunity in NZB/W mice. The Journal of experimental medicine 199, 1577-1584.
Hu, C. Y., Rodriguez-Pinto, D., Du, W., Ahuja, A., Henegariu, O., Wong, F. S., Shlomchik, M. J., and Wen, L. (2007). Treatment with CD20-specific antibody prevents and reverses autoimmune diabetes in mice. The Journal of clinical investigation 117, 3857-3867.
Hughes, E. G., Peng, X., Gleichman, A. J., Lai, M., Zhou, L., Tsou, R., Parsons, T. D., Lynch, D. R., Dalmau, J., and Balice-Gordon, R. J. (2010). Cellular and synaptic mechanisms of anti-NMDA receptor encephalitis. The Journal of neuroscience: the official journal of the Society for Neuroscience 30, 5866-5875.
Huijbers, M. G., Lipka, A. F., Plomp, J. J., Niks, E. H., van der Maarel, S. M., and Verschuuren, J. J. (2014). Pathogenic immune mechanisms at the neuromuscular synapse: the role of specific antibody-binding epitopes in myasthenia gravis. Journal of internal medicine 275, 12-26.
Hull, C. M., Liddle, M., Hansen, N., Meyer, L. J., Schmidt, L., Taylor, T., Jaskowski, T. D., Hill, H. R., and Zone, J. J. (2008). Elevation of IgA anti-epidermal transglutaminase antibodies in dermatitis herpetiformis. The British journal of dermatology 159, 120-124.
Humby, F., Bombardieri, M., Manzo, A., Kelly, S., Blades, M. C., Kirkham, B., Spencer, J., and Pitzalis, C. (2009). Ectopic lymphoid structures support ongoing production of class-switched autoantibodies in rheumatoid synovium. PLoS medicine 6, el.
Hung, G. C., Liu, H. C., Yang, S. Y., Pan, C. H., Liao, Y. T., Chen, C. C., and Kuo, C. J. (2016). Antipsychotic reexposure and recurrent pneumonia in schizophrenia: a nested case-control study. The Journal of clinical psychiatry 77, 60-66.
Inoue, T., Moran, I., Shinnakasu, R., Phan, T. G., and Kurosaki, T. (2018). Generation of memory B cells and their reactivation. Immunological reviews 283, 138-149.
Ishii, K., Amagai, M., Hall, R. P., Hashimoto, T., Takayanagi, A., Gamou, S., Shimizu, N., and Nishikawa, T. (1997). Characterization of autoantibodies in pemphigus using antigen-specific enzyme-linked immunosorbent assays with baculovirus-expressed recombinant desmogleins. Journal of immunology (Baltimore, Md.: 1950) 159, 2010-2017.
Ishisaka, M., Tsujii, S., Mizoguchi, T., Tsuruma, K., Shimazawa, M., and Hara, H. (2015). The effects of valproate and olanzapine on the abnormal behavior of diacylglycerol kinase beta knockout mice. Pharmacological reports: PR 67, 275-280.
Islam, N., Leung, P. S., Huntley, A. C., and Gershwin, M. E. (2015). The autoimmune basis of alopecia areata: a comprehensive review. Autoimmunity reviews 14, 81-89.
Iwata, Y., Wada, T., Uchiyama, A., Miwa, A., Nakaya, I., Tohyama, T., Yamada, Y., Kurokawa, T., Yoshida, T., Ohta, S., et al. (2006). Remission of IgA nephropathy after allogeneic peripheral blood stem cell transplantation followed by immunosuppression for acute lymphocytic leukemia. Internal medicine (Tokyo, Japan) 45, 1291-1295.
Jelcic, I., Al Nimer, F., Wang, J., Lentsch, V., Planas, R., Jelcic, I., Madjovski, A., Ruhrmann, S., Faigle, W., Frauenknecht, K., et al. (2018). Memory B Cells Activate Brain-Homing, Autoreactive CD4(+) T Cells in Multiple Sclerosis. Cell 175, 85-100.e123.
Jensen, D. M., McFarlane, I. G., Portmann, B. S., Eddleston, A. L., and Williams, R. (1978). Detection of antibodies directed against a liver-specific membrane lipoprotein in patients with acute and chronic active hepatitis. The New England journal of medicine 299, 1-7.
Jolles, S., Borrell, R., Zouwail, S., Heaps, A., Sharp, H., Moody, M., Selwood, C., Williams, P., Phillips, C., Hood, K., et al. (2014). Calculated globulin (CG) as a screening test for antibody deficiency. Clinical and experimental immunology 177, 671-678.
Joly, P., Mouquet, H., Roujeau, J. C., D'Incan, M., Gilbert, D., Jacquot, S., Gougeon, M. L., Bedane, C., Muller, R., Dreno, B., et al. (2007). A single cycle of rituximab for the treatment of severe pemphigus. The New England journal of medicine 357, 545-552.
Joshi, R. S., Quadros, R., Drumm, M., Ain, R., and Panicker, M. M. (2017). Sedative effect of Clozapine is a function of 5-HT2A and environmental novelty. European neuropsychopharmacology: the journal of the European College of Neuropsychopharmacology 27, 70-81.
Kaplan, A. P., Gimenez-Arnau, A. M., and Saini, S. S. (2017). Mechanisms of action that contribute to efficacy of omalizumab in chronic spontaneous urticaria. Allergy 72, 519-533.
Kappos, L., Hartung, H. P., Freedman, M. S., Boyko, A., Radu, E. W., Mikol, D. D., Lamarine, M., Hyvert, Y., Freudensprung, U., Plitz, T., et al. (2014). Atacicept in multiple sclerosis (ATAMS): a randomised, placebo-controlled, double-blind, phase 2 trial. The Lancet Neurology 13, 353-363.
Karlsen, T. H., Folseraas, T., Thorburn, D., and Vesterhus, M. (2017). Primary sclerosing cholangitis—a comprehensive review. Journal of hepatology 67, 1298-1323.
Karpatkin, S., and Siskind, G. W. (1969). In vitro detection of platelet antibody in patients with idiopathic thrombocytopenic purpura and systemic lupus erythematosus. Blood 33, 795-812.
Kasperkiewicz, M., Ellebrecht, C. T., Takahashi, H., Yamagami, J., Zillikens, D., Payne, A. S., and Amagai, M. (2017). Pemphigus. Nature reviews Disease primers 3, 17026.
Kasperkiewicz, M., Meier, M., Zillikens, D., and Schmidt, E. (2010). Linear IgA disease: successful application of immunoadsorption and review of the literature. Dermatology (Basel, Switzerland) 220, 259-263.
Keegan, M., Konig, F., McClelland, R., Bruck, W., Morales, Y., Bitsch, A., Panitch, H., Lassmann, H., Weinshenker, B., Rodriguez, M., et al. (2005). Relation between humoral pathological changes in multiple sclerosis and response to therapeutic plasma exchange. Lancet (London, England) 366, 579-582.
Kemp, E. H., Gavalas, N. G., Gawkrodger, D. J., and Weetman, A. P. (2007). Autoantibody responses to melanocytes in the depigmenting skin disease vitiligo. Autoimmunity reviews 6, 138-142.
Kemp, E. H., Waterman, E. A., Hawes, B. E., O'Neill, K., Gottumukkala, R. V., Gawkrodger, D. J., Weetman, A. P., and Watson, P. F. (2002). The melanin-concentrating hormone receptor 1, a novel target of autoantibody responses in vitiligo. The Journal of clinical investigation 109, 923-930.
Kessel, A., Helou, W., Bamberger, E., Sabo, E., Nusem, D., Panassof, J., and Toubi, E. (2010). Elevated serum total IgE—a potential marker for severe chronic urticaria. International archives of allergy and immunology 153, 288-293.
Kessel, A., Yaacoby-Bianu, K., Vadasz, Z., Peri, R., Halazs, K., and Toubi, E. (2012). Elevated serum B-cell activating factor in patients with chronic urticaria. Human immunology 73, 620-622.
Khellaf, M., Charles-Nelson, A., Fain, O., Terriou, L., Viallard, J. F., Cheze, S., Graveleau, J., Slama, B., Audia, S., Ebbo, M., et al. (2014). Safety and efficacy of rituximab in adult immune thrombocytopenia: results from a prospective registry including 248 patients. Blood 124, 3228-3236.
Khoury, E. L., Hammond, L., Bottazzo, G. F., and Doniach, D. (1981). Surface-reactive antibodies to human adrenal cells in Addison's disease. Clinical and experimental immunology 45, 48-55.
Kikuchi, Y., and Kaplan, A. P. (2002). A role for C5a in augmenting IgG-dependent histamine release from basophils in chronic urticaria. The Journal of allergy and clinical immunology 109, 114-118.
Kirtschig, G., and Wojnarowska, F. (1999). IgA basement membrane zone autoantibodies in bullous pemphigoid detect epidermal antigens of 270-280 kDa, 230 kDa, and 180 kDa molecular weight by immunoblotting. Clinical and experimental dermatology 24, 302-307.
Kleinau, S., Martinsson, P., and Heyman, B. (2000). Induction and suppression of collagen-induced arthritis is dependent on distinct fcgamma receptors. The Journal of experimental medicine 191, 1611-1616.
Knight, G. B., Gao, L., Gragnani, L., Elfahal, M. M., De Rosa, F. G., Gordon, F. D., and Agnello, V. (2010). Detection of WA B cells in hepatitis C virus infection: a potential prognostic marker for cryoglobulinemic vasculitis and B cell malignancies. Arthritis and rheumatism 62, 2152-2159.
Kohler, S., Keil, T. O., Swierzy, M., Hoffmann, S., Schaffert, H., Ismail, M., Ruckert, J. C., Alexander, T., Hiepe, F., Gross, C., et al. (2013). Disturbed B cell subpopulations and increased plasma cells in myasthenia gravis patients. Journal of neuroimmunology 264, 114-119.
Kolkhir, P., Church, M. K., Weller, K., Metz, M., Schmetzer, O., and Maurer, M. (2017). Autoimmune chronic spontaneous urticaria: What we know and what we do not know. The Journal of allergy and clinical immunology 139, 1772-1781.e1771.
Kracker, S., and Durandy, A. (2011). Insights into the B cell specific process of immunoglobulin class switch recombination. Immunology letters 138, 97-103.
Krumbholz, M., Derfuss, T., Hohlfeld, R., and Meinl, E. (2012). B cells and antibodies in multiple sclerosis pathogenesis and therapy. Nature reviews Neurology 8, 613-623.
Kuenz, B., Lutterotti, A., Ehling, R., Gneiss, C., Haemmerle, M., Rainer, C., Deisenhammer, F., Schocke, M., Berger, T., and Reindl, M. (2008). Cerebrospinal fluid B cells correlate with early brain inflammation in multiple sclerosis. PloS one 3, e2559.
Kumar, V., Gutierrez-Achury, J., Kanduri, K., Almeida, R., Hrdlickova, B., Zhernakova, D. V., Westra, H. J., Karjalainen, J., Ricano-Ponce, I., Li, Y., et al. (2015). Systematic annotation of celiac disease loci refines pathological pathways and suggests a genetic explanation for increased interferon-gamma levels. Human molecular genetics 24, 397-409.
Kuo, C. J., Yang, S. Y., Liao, Y. T., Chen, W. J., Lee, W. C., Shau, W. Y., Chang, Y. T., Tsai, S. Y., and Chen, C. C. (2013). Second-generation antipsychotic medications and risk of pneumonia in schizophrenia. Schizophrenia bulletin 39, 648-657.
Lafayette, R. A., Canetta, P. A., Rovin, B. H., Appel, G. B., Novak, J., Nath, K. A., Sethi, S., Tumlin, J. A., Mehta, K., Hogan, M., et al. (2017). A Randomized, Controlled Trial of Rituximab in IgA Nephropathy with Proteinuria and Renal Dysfunction. Journal of the American Society of Nephrology: JASN 28, 1306-1313.
Lai, K. N., Tang, S. C., Schena, F. P., Novak, J., Tomino, Y., Fogo, A. B., and Glassock, R. J. (2016). IgA nephropathy. Nature reviews Disease primers 2, 16001.
Lamberts, A., Euverman, H. I., Terra, J. B., Jonkman, M. F., and Horvath, B. (2018). Effectiveness and Safety of Rituximab in Recalcitrant Pemphigoid Diseases. Frontiers in immunology 9, 248.
Lancaster-Smith, M., Joyce, S., and Kumar, P. (1977). Immunoglobulins in the jejunal mucosa in adult coeliac disease and dermatitis herpetiformis after the reintroduction of dietary gluten. Gut 18, 887-891.
Lancaster, E., Lai, M., Peng, X., Hughes, E., Constantinescu, R., Raizer, J., Friedman, D., Skeen, M. B., Grisold, W., Kimura, A., et al. (2010). Antibodies to the GABA(B) receptor in limbic encephalitis with seizures: case series and characterisation of the antigen. The Lancet Neurology 9, 67-76.
Laszlo, S., Neumann, S., Hertl, M., and Hunzelmann, N. (2010). Generation of increased numbers of HLA-DR(high) IgG+ plasma cells in the peripheral blood of patients with bullous pemphigoid: NC16a-specific cells belong to the short-lived plasma blast population. The Journal of investigative dermatology 130, 2838-2841.
Laureti, S., De Bellis, A., Muccitelli, V. I., Calcinaro, F., Bizzarro, A., Rossi, R., Bellastella, A., Santeusanio, F., and Falorni, A. (1998). Levels of adrenocortical autoantibodies correlate with the degree of adrenal dysfunction in subjects with preclinical Addison's disease. The Journal of clinical endocrinology and metabolism 83, 3507-3511.
Lee, J. Y., Stathopoulos, P., Gupta, S., Bannock, J. M., Barohn, R. J., Cotzomi, E., Dimachkie, M. M., Jacobson, L., Lee, C. S., Morbach, H., et al. (2016a). Compromised fidelity of B-cell tolerance checkpoints in AChR and MuSK myasthenia gravis. Annals of clinical and translational neurology 3, 443-454.
Lee, W. J., Lee, S. T., Byun, J. I., Sunwoo, J. S., Kim, T. J., Lim, J. A., Moon, J., Lee, H. S., Shin, Y. W., Lee, K. J., et al. (2016b). Rituximab treatment for autoimmune limbic encephalitis in an institutional cohort. Neurology 86, 1683-1691.
Leprince, C., Cohen-Kaminsky, S., Berrih-Aknin, S., Vernet-Der Garabedian, B., Treton, D., Galanaud, P., and Richard, Y. (1990). Thymic B cells from myasthenia gravis patients are activated B cells. Phenotypic and functional analysis. Journal of immunology (Baltimore, Md.: 1950) 145, 2115-2122.
Leung, J. G., Hasassri, M. E., Barreto, J. N., Nelson, S., and Morgan, R. J., 3rd (2017). Characterization of Admission Types in Medically Hospitalized Patients Prescribed Clozapine. Psychosomatics 58, 164-172.
Leyendeckers, H., Tasanen, K., Bruckner-Tuderman, L., Zillikens, D., Sitaru, C., Schmitz, J., and Hunzelmann, N. (2003). Memory B cells specific for the NC16A domain of the 180 kDa bullous pemphigoid autoantigen can be detected in peripheral blood of bullous pemphigoid patients and induced in vitro to synthesize autoantibodies. The Journal of investigative dermatology 120, 372-378.
Li, H., Fang, M., Xu, M., Li, S., Du, J., Li, W., and Chen, H. (2016a). Chronic Olanzapine Treatment Induces Disorders of Plasma Fatty Acid Profile in Balb/c Mice: A Potential Mechanism for Olanzapine-Induced Insulin Resistance. PloS one 11, e0167930.
Li, Y., Foshee, J. B., and Sontheimer, R. D. (2011). Sustained clinical response to rituximab in a case of life-threatening overlap subepidermal autoimmune blistering disease. Journal of the American Academy of Dermatology 64, 773-778.
Li, Y., Yang, M., Zhang, R., Liu, W., Zhang, K., Wen, W., Yi, L., Wang, Q., Hao, M., Yang, H., et al. (2016b). Evaluation of serum immunoglobulins concentrations and distributions in vitiligo patients. Immunologic research 64, 1150-1156.
Liberal, R., Mieli-Vergani, G., and Vergani, D. (2013). Clinical significance of autoantibodies in autoimmune hepatitis. Journal of autoimmunity 46, 17-24.
Ligocki, A. J., Rounds, W. H., Cameron, E. M., Harp, C. T., Frohman, E. M., Courtney, A. M., Vernino, S., Cowell, L. G., Greenberg, B., and Monson, N. L. (2013). Expansion of CD27high plasmablasts in transverse myelitis patients that utilize VH4 and JH6 genes and undergo extensive somatic hypermutation. Genes and immunity 14, 291-301.
Lindfors, K., Ciacci, C., Kurppa, K., Lundin, K. E. A., Makharia, G. K., Mearin, M. L., Murray, J. A., Verdu, E. F., and Kaukinen, K. (2019). Coeliac disease. Nature reviews Disease primers 5, 3.
Liu, C. W., and Huang, Y. C. (2018). Vitiligo and autoantibodies: a systematic review and meta-analysis. Journal der Deutschen Dermatologischen Gesellschaft=Journal of the German Society of Dermatology: JDDG 16, 845-851.
LoBuglio, A. F., Cotran, R. S., and Jandl, J. H. (1967). Red cells coated with immunoglobulin G: binding and sphering by mononuclear cells in man. Science (New York, N.Y.) 158, 1582-1585.
Longbrake, E. E., and Cross, A. H. (2016). Effect of Multiple Sclerosis Disease-Modifying Therapies on B Cells and Humoral Immunity. JAMA neurology 73, 219-225.
Lovato, L., Willis, S. N., Rodig, S. J., Caron, T., Almendinger, S. E., Howell, O. W., Reynolds, R., O'Connor, K. C., and Hafler, D. A. (2011). Related B cell clones populate the meninges and parenchyma of patients with multiple sclerosis. Brain: a journal of neurology 134, 534-541.
Lozano, R., Marin, R., Santacruz, M. J., and Pascual, A. (2016). Effect of clozapine on immunoglobulin M plasma levels. Therapeutic advances in psychopharmacology 6, 58-60.
Ma, L., Qin, J., Ji, H., Zhao, P., and Jiang, Y. (2014). Tfh and plasma cells are correlated with hypergammaglobulinaemia in patients with autoimmune hepatitis. Liver international: official journal of the International Association for the Study of the Liver 34, 405-415.
Ma, Y., Okamoto, M., Thomas, M. G., Bogdanos, D. P., Lopes, A. R., Portmann, B., Underhill, J., Durr, R., Mieli-Vergani, G., and Vergani, D. (2002). Antibodies to conformational epitopes of soluble liver antigen define a severe form of autoimmune liver disease. Hepatology (Baltimore, Md.) 35, 658-664.
MacGlashan, D. W., Jr., Bochner, B. S., Adelman, D. C., Jardieu, P. M., Togias, A., McKenzie-White, J., Sterbinsky, S. A., Hamilton, R. G., and Lichtenstein, L. M. (1997). Down-regulation of Fc(epsilon)RI expression on human basophils during in vivo treatment of atopic patients with anti-IgE antibody. Journal of immunology (Baltimore, Md.: 1950) 158, 1438-1445.
Mahevas, M., Michel, M., Vingert, B., Moroch, J., Boutboul, D., Audia, S., Cagnard, N., Ripa, J., Menard, C., Tarte, K., et al. (2015). Emergence of long-lived autoreactive plasma cells in the spleen of primary warm auto-immune hemolytic anemia patients treated with rituximab. Journal of autoimmunity 62, 22-30.
Mahevas, M., Patin, P., Huetz, F., Descatoire, M., Cagnard, N., Bole-Feysot, C., Le Gallou, S., Khellaf, M., Fain, O., Boutboul, D., et al. (2013). B cell depletion in immune thrombocytopenia reveals splenic long-lived plasma cells. The Journal of clinical investigation 123, 432-442.
Makino, T., Nakamura, R., Terakawa, M., Muneoka, S., Nagahira, K., Nagane, Y., Yamate, J., Motomura, M., and Utsugisawa, K. (2017). Analysis of peripheral B cells and autoantibodies against the anti-nicotinic acetylcholine receptor derived from patients with myasthenia gravis using single-cell manipulation tools. PloS one 12, e0185976.
Malviya, M., Barman, S., Golombeck, K. S., Planaguma, J., Mannara, F., Strutz-Seebohm, N., Wrzos, C., Demir, F., Baksmeier, C., Steckel, J., et al. (2017). NMDAR encephalitis: passive transfer from man to mouse by a recombinant antibody. Annals of clinical and translational neurology 4, 768-783.
Marinkovic, D., Timotijevic, I., Babinski, T., Totic, S., and Paunovic, V. R. (1994). The side-effects of clozapine: a four year follow-up study. Progress in neuro-psychopharmacology & biological psychiatry 18, 537-544.
Marino, E., Tan, B., Binge, L., Mackay, C. R., and Grey, S. T. (2012). B-cell cross-presentation of autologous antigen precipitates diabetes. Diabetes 61, 2893-2905.
Marker, M., Derfler, K., Monshi, B., and Rappersberger, K. (2011). Successful immunoapheresis of bullous autoimmune diseases: Pemphigus vulgaris and pemphigoid gestationis. Journal der Deutschen Dermatologischen Gesellschaft=Journal of the German Society of Dermatology: JDDG 9, 27-31.
Mathey, E. K., Derfuss, T., Storch, M. K., Williams, K. R., Hales, K., Woolley, D. R., Al-Hayani, A., Davies, S. N., Rasband, M. N., Olsson, T., et al. (2007). Neurofascin as a novel target for autoantibody-mediated axonal injury. The Journal of experimental medicine 204, 2363-2372.
Matsumoto, M., Baba, A., Yokota, T., Nishikawa, H., Ohkawa, Y., Kayama, H., Kallies, A., Nutt, S. L., Sakaguchi, S., Takeda, K., et al. (2014). Interleukin-10-producing plasmablasts exert regulatory function in autoimmune inflammation. Immunity 41, 1040-1051.
Matysiak-Budnik, T., Moura, I. C., Arcos-Fajardo, M., Lebreton, C., Menard, S., Candalh, C., Ben-Khalifa, K., Dugave, C., Tamouza, H., van Niel, G., et al. (2008). Secretory IgA mediates retrotranscytosis of intact gliadin peptides via the transferrin receptor in celiac disease. The Journal of experimental medicine 205, 143-154.
Mayer, M. C., and Meinl, E. (2012). Glycoproteins as targets of autoantibodies in CNS inflammation: MOG and more. Therapeutic advances in neurological disorders 5, 147-159.
McElwee, K. J., Gilhar, A., Tobin, D. J., Ramot, Y., Sundberg, J. P., Nakamura, M., Bertolini, M., Inui, S., Tokura, Y., King, L. E., Jr., et al. (2013). What causes alopecia areata? Experimental dermatology 22, 609-626.
McKnight, K., Jiang, Y., Hart, Y., Cavey, A., Wroe, S., Blank, M., Shoenfeld, Y., Vincent, A., Palace, J., and Lang, B. (2005). Serum antibodies in epilepsy and seizure-associated disorders. Neurology 65, 1730-1736.
McMillan, R., Luiken, G. A., Levy, R., Yelenosky, R., and Longmire, R. L. (1978). Antibody against megakaryocytes in idiopathic thrombocytopenic purpura. Jama 239, 2460-2462.
McMillan, R., Wang, L., and Tani, P. (2003). Prospective evaluation of the immunobead assay for the diagnosis of adult chronic immune thrombocytopenic purpura (ITP). Journal of thrombosis and haemostasis: JTH 1, 485-491.
McMillan, R., Wang, L., Tomer, A., Nichol, J., and Pistillo, J. (2004). Suppression of in vitro megakaryocyte production by antiplatelet autoantibodies from adult patients with chronic ITP. Blood 103, 1364-1369.
McOmish, C. E., Lira, A., Hanks, J. B., and Gingrich, J. A. (2012). Clozapine-induced locomotor suppression is mediated by 5-HT2A receptors in the forebrain. Neuropsychopharmacology: official publication of the American College of Neuropsychopharmacology 37, 2747-2755.
Mei, H. E., Yoshida, T., Sime, W., Hiepe, F., Thiele, K., Manz, R. A., Radbruch, A., and Dorner, T. (2009). Blood-borne human plasma cells in steady state are derived from mucosal immune responses. Blood 113, 2461-2469.
Melchers, F. (2015). Checkpoints that control B cell development. The Journal of clinical investigation 125, 2203-2210.
Meyer-Hermann, M., Mohr, E., Pelletier, N., Zhang, Y., Victora, G. D., and Toellner, K. M. (2012). A theory of germinal center B cell selection, division, and exit. Cell reports 2, 162-174.
Michel, L., Touil, H., Pikor, N. B., Gommerman, J. L., Prat, A., and Bar-Or, A. (2015). B Cells in the Multiple Sclerosis Central Nervous System: Trafficking and Contribution to CNS-Compartmentalized Inflammation. Frontiers in immunology 6, 636.
Migita, K., Abiru, S., Maeda, Y., Nakamura, M., Komori, A., Ito, M., Fujiwara, S., Yano, K., Yatsuhashi, H., Eguchi, K., et al. (2007). Elevated serum BAFF levels in patients with autoimmune hepatitis. Human immunology 68, 586-591.
Millan, M. J., Audinot, V., Melon, C., and Newman-Tancredi, A. (1995). Evidence that dopamine D3 receptors participate in clozapine-induced hypothermia. European journal of pharmacology 280, 225-229.
Mitchell, A. L., and Pearce, S. H. (2012). Autoimmune Addison disease: pathophysiology and genetic complexity. Nature reviews Endocrinology 8, 306-316.
Montalban, X., Hauser, S. L., Kappos, L., Arnold, D. L., Bar-Or, A., Comi, G., de Seze, J., Giovannoni, G., Hartung, H. P., Hemmer, B., et al. (2017). Ocrelizumab versus Placebo in Primary Progressive Multiple Sclerosis. The New England journal of medicine 376, 209-220.
Moreno, J. L., Holloway, T., Umali, A., Rayannavar, V., Sealfon, S. C., and Gonzalez-Maeso, J. (2013). Persistent effects of chronic clozapine on the cellular and behavioral responses to LSD in mice. Psychopharmacology 225, 217-226.
Moschovakis, G. L., Bubke, A., Friedrichsen, M., Falk, C. S., Feederle, R., and Forster, R. (2017). T cell specific Cxcr5 deficiency prevents rheumatoid arthritis. Scientific reports 7, 8933.
Mumtaz, I. M., Hoyer, B. F., Panne, D., Moser, K., Winter, O., Cheng, Q. Y., Yoshida, T., Burmester, G. R., Radbruch, A., Manz, R. A., et al. (2012). Bone marrow of NZB/W mice is the major site for plasma cells resistant to dexamethasone and cyclophosphamide: implications for the treatment of autoimmunity. Journal of autoimmunity 39, 180-188.
Mutlu, O., Gumuslu, E., Ulak, G., Celikyurt, I. K., Kokturk, S., Kir, H. M., Akar, F., and Erden, F. (2012). Effects of fluoxetine, tianeptine and olanzapine on unpredictable chronic mild stress-induced depression-like behavior in mice. Life sciences 91, 1252-1262.
Muto, A., Ochiai, K., Kimura, Y., Itoh-Nakadai, A., Calame, K. L., Ikebe, D., Tashiro, S., and Igarashi, K. (2010). Bach2 represses plasma cell gene regulatory network in B cells to promote antibody class switch. The EMBO journal 29, 4048-4061.
Muto, A., Tashiro, S., Nakajima, O., Hoshino, H., Takahashi, S., Sakoda, E., Ikebe, D., Yamamoto, M., and Igarashi, K. (2004). The transcriptional programme of antibody class switching involves the repressor Bach2. Nature 429, 566-571.
Muto, M., Manfroi, B., Suzuki, H., Joh, K., Nagai, M., Wakai, S., Righini, C., Maiguma, M., Izui, S., Tomino, Y., et al. (2017). Toll-Like Receptor 9 Stimulation Induces Aberrant Expression of a Proliferation-Inducing Ligand by Tonsillar Germinal Center B Cells in IgA Nephropathy. Journal of the American Society of Nephrology: JASN 28, 1227-1238.
Myers, R. P., Swain, M. G., Lee, S. S., Shaheen, A. A., and Burak, K. W. (2013). B-cell depletion with rituximab in patients with primary biliary cirrhosis refractory to ursodeoxycholic acid. The American journal of gastroenterology 108, 933-941.
Myrsky, E., Kaukinen, K., Syrjanen, M., Korponay-Szabo, I. R., Maki, M., and Lindfors, K. (2008). Coeliac disease-specific autoantibodies targeted against transglutaminase 2 disturb angiogenesis. Clinical and experimental immunology 152, 111-119.
Naito, Y., Takematsu, H., Koyama, S., Miyake, S., Yamamoto, H., Fujinawa, R., Sugai, M., Okuno, Y., Tsujimoto, G., Yamaji, T., et al. (2007). Germinal center marker GL7 probes activation-dependent repression of N-glycolylneuraminic acid, a sialic acid species involved in the negative modulation of B-cell activation. Molecular and cellular biology 27, 3008-3022.
Nakamura, M. (2014). Clinical significance of autoantibodies in primary biliary cirrhosis. Seminars in liver disease 34, 334-340.
Nandakumar, K. S., Backlund, J., Vestberg, M., and Holmdahl, R. (2004). Collagen type II (CII)-specific antibodies induce arthritis in the absence of T or B cells but the arthritis progression is enhanced by CII-reactive T cells. Arthritis research & therapy 6, R544-550.
Nguyen Canh, H., Harada, K., Ouchi, H., Sato, Y., Tsuneyama, K., Kage, M., Nakano, M., Yoshizawa, K., Takahashi, A., Abe, M., et al. (2017). Acute presentation of autoimmune hepatitis: a multicentre study with detailed histological evaluation in a large cohort of patients. Journal of clinical pathology 70, 961-969.
Nguyen, D. C., Garimalla, S., Xiao, H., Kyu, S., Albizua, I., Galipeau, J., Chiang, K. Y., Waller, E. K., Wu, R., Gibson, G., et al. (2018). Factors of the bone marrow microniche that support human plasma cell survival and immunoglobulin secretion. Nature communications 9, 3698.
Niimi, N., Francis, D. M., Kermani, F., O'Donnell, B. F., Hide, M., Kobza-Black, A., Winkelmann, R. K., Greaves, M. W., and Barr, R. M. (1996). Dermal mast cell activation by autoantibodies against the high affinity IgE receptor in chronic urticaria. The Journal of investigative dermatology 106, 1001-1006.
Nikiphorou, E., and Hall, F. C. (2014). First report of improvement of coeliac disease in a patient with Sjogren's syndrome treated with rituximab. Rheumatology (Oxford, England) 53, 1906-1907.
Noorchashm, H., Noorchashm, N., Kern, J., Rostami, S. Y., Barker, C. F., and Naji, A. (1997). B-cells are required for the initiation of insulitis and sialitis in nonobese diabetic mice. Diabetes 46, 941-946.
Norris, D. A., Kissinger, R. M., Naughton, G. M., and Bystryn, J. C. (1988). Evidence for immunologic mechanisms in human vitiligo: patients' sera induce damage to human melanocytes in vitro by complement-mediated damage and antibody-dependent cellular cytotoxicity. The Journal of investigative dermatology 90, 783-789.
Nugent, D., McMillan, R., Nichol, J. L., and Slichter, S. J. (2009). Pathogenesis of chronic immune thrombocytopenia: increased platelet destruction and/or decreased platelet production. British journal of haematology 146, 585-596.
Nutt, S. L., Hodgkin, P. D., Tarlinton, D. M., and Corcoran, L. M. (2015). The generation of antibody-secreting plasma cells. Nature reviews Immunology 15, 160-171.
Obermeier, B., Mentele, R., Malotka, J., Kellermann, J., Kumpfel, T., Wekerle, H., Lottspeich, F., Hohlfeld, R., and Dornmair, K. (2008). Matching of oligoclonal immunoglobulin transcriptomes and proteomes of cerebrospinal fluid in multiple sclerosis. Nature medicine 14, 688-693.
Orange, J. S., Ballow, M., Stiehm, E. R., Ballas, Z. K., Chinen, J., De La Morena, M., Kumararatne, D., Harville, T. O., Hesterberg, P., Koleilat, M., et al. (2012). Use and interpretation of diagnostic vaccination in primary immunodeficiency: a working group report of the Basic and Clinical Immunology Interest Section of the American Academy of Allergy, Asthma & Immunology. The Journal of allergy and clinical immunology 130, S1-24.
Osborne, D., and Sobczynska-Malefora, A. (2015). Autoimmune mechanisms in pernicious anaemia & thyroid disease. Autoimmunity reviews 14, 763-768.
Owczarczyk, K., Lal, P., Abbas, A. R., Wolslegel, K., Holweg, C. T., Dummer, W., Kelman, A., Brunetta, P., Lewin-Koh, N., Sorani, M., et al. (2011). A plasmablast biomarker for nonresponse to antibody therapy to CD20 in rheumatoid arthritis. Science translational medicine 3, 101ra192.
Pabst, O. (2012). New concepts in the generation and functions of IgA. Nature reviews Immunology 12, 821-832.
Palanichamy, A., Apeltsin, L., Kuo, T. C., Sirota, M., Wang, S., Pitts, S. J., Sundar, P. D., Telman, D., Zhao, L. Z., Derstine, M., et al. (2014). Immunoglobulin class-switched B cells form an active immune axis between CNS and periphery in multiple sclerosis. Science translational medicine 6, 248ra106.
Parackova, Z., Klocperk, A., Rataj, M., Kayserova, J., Zentsova, I., Sumnik, Z., Kolouskova, S., Sklenarova, J., Pruhova, S., Obermannova, B., et al. (2017). Alteration of B cell subsets and the receptor for B cell activating factor (BAFF) in paediatric patients with type 1 diabetes. Immunology letters 189, 94-100.
Payet, J., Livartowski, J., Kavian, N., Chandesris, O., Dupin, N., Wallet, N., Karras, A., Salliot, C., Suarez, F., Avet-Loiseau, H., et al. (2013). Type I cryoglobulinemia in multiple myeloma, a rare entity: analysis of clinical and biological characteristics of seven cases and review of the literature. Leukemia & lymphoma 54, 767-777.
Pazderska, A., Oftedal, B. E., Napier, C. M., Ainsworth, H. F., Husebye, E. S., Cordell, H. J., Pearce, S. H., and Mitchell, A. L. (2016). A Variant in the BACH2 Gene Is Associated With Susceptibility to Autoimmune Addison's Disease in Humans. The Journal of clinical endocrinology and metabolism 101, 3865-3869.
Pearce, S. H., Mitchell, A. L., Bennett, S., King, P., Chandran, S., Nag, S., Chen, S., Smith, B. R., Isaacs, J. D., and Vaidya, B. (2012). Adrenal steroidogenesis after B lymphocyte depletion therapy in new-onset Addison's disease. The Journal of clinical endocrinology and metabolism 97, E1927-1932.
Pescovitz, M. D., Greenbaum, C. J., Krause-Steinrauf, H., Becker, D. J., Gitelman, S. E., Goland, R., Gottlieb, P. A., Marks, J. B., McGee, P. F., Moran, A. M., et al. (2009). Rituximab, B-lymphocyte depletion, and preservation of beta-cell function. The New England journal of medicine 361, 2143-2152.
Petro, J. B., Gerstein, R. M., Lowe, J., Carter, R. S., Shinners, N., and Khan, W. N. (2002). Transitional type 1 and 2 B lymphocyte subsets are differentially responsive to antigen receptor signaling. The Journal of biological chemistry 277, 48009-48019.
Pinard, C., Hebert, V., Lecuyer, M., Sacre, L., and Joly, P. (2019). Linear IgA bullous dermatosis treated with rituximab. JAAD case reports 5, 124-126.
Placzek, W. J., Yanagawa, H., Makita, Y., Renfrow, M. B., Julian, B. A., Rizk, D. V., Suzuki, Y., Novak, J., and Suzuki, H. (2018). Serum galactose-deficient-IgA1 and IgG autoantibodies correlate in patients with IgA nephropathy. PloS one 13, e0190967.
Planaguma, J., Leypoldt, F., Mannara, F., Gutierrez-Cuesta, J., Martin-Garcia, E., Aguilar, E., Titulaer, M. J., Petit-Pedrol, M., Jain, A., Balice-Gordon, R., et al. (2015). Human N-methyl D-aspartate receptor antibodies alter memory and behaviour in mice. Brain: a journal of neurology 138, 94-109.
Ponsford, M., Castle, D., Tahir, T., Robinson, R., Wade, W., Steven, R., Bramhall, K., Moody, M., Carne, E., Ford, C., et al. (2018). Clozapine is associated with secondary antibody deficiency. The British journal of psychiatry: the journal of mental science, 1-7.
Qian, H., Kusuhara, M., Li, X., Tsuruta, D., Tsuchisaka, A., Ishii, N., Koga, H., Hayakawa, T., Ohara, K., Karashima, T., et al. (2014). B-cell activating factor detected on both naive and memory B cells in bullous pemphigoid. Experimental dermatology 23, 596-605.
Quek, A. M., Britton, J. W., McKeon, A., So, E., Lennon, V. A., Shin, C., Klein, C., Watson, R. E., Jr., Kotsenas, A. L., Lagerlund, T. D., et al. (2012). Autoimmune epilepsy: clinical characteristics and response to immunotherapy. Archives of neurology 69, 582-593.
Ramos-Casals, M., Stone, J. H., Cid, M. C., and Bosch, X. (2012). The cryoglobulinaemias. Lancet (London, England) 379, 348-360.
Ranki, A., Kianto, U., Kanerva, L., Tolvanen, E., and Johansson, E. (1984). Immunohistochemical and electron microscopic characterization of the cellular infiltrate in alopecia (areata, totalis, and universalis). The Journal of investigative dermatology 83, 7-11.
Richez, C., Truchetet, M. E., Schaeverbeke, T., and Bannwarth, B. (2014). Atacicept as an investigated therapy for rheumatoid arthritis. Expert opinion on investigational drugs 23, 1285-1294.
Rivas, J. R., Ireland, S. J., Chkheidze, R., Rounds, W. H., Lim, J., Johnson, J., Ramirez, D. M., Ligocki, A. J., Chen, D., Guzman, A. A., et al. (2017). Peripheral VH4+ plasmablasts demonstrate autoreactive B cell expansion toward brain antigens in early multiple sclerosis patients. Acta neuropathologica 133, 43-60.
Rockx, M. A., and Clark, W. F. (2010). Plasma exchange for treating cryoglobulinemia: a descriptive analysis. Transfusion and apheresis science: official journal of the World Apheresis Association: official journal of the European Society for Haemapheresis 42, 247-251.
Rojas, O. L., Probstel, A. K., Porfilio, E. A., Wang, A. A., Charabati, M., Sun, T., Lee, D. S. W., Galicia, G., Ramaglia, V., Ward, L. A., et al. (2019). Recirculating Intestinal IgA-Producing Cells Regulate Neuroinflammation via IL-10. Cell 176, 610-624.e618.
Rostom, A., Dube, C., Cranney, A., Saloojee, N., Sy, R., Garritty, C., Sampson, M., Zhang, L., Yazdi, F., Mamaladze, V., et al. (2005). The diagnostic accuracy of serologic tests for celiac disease: a systematic review. Gastroenterology 128, S38-46.
Rowley, M. J., and Whittingham, S. F. (2015). The Role of Pathogenic Autoantibodies in Autoimmunity. Antibodies 4, 314.
Ruach, M., Ohel, G., Rahav, D., and Samueloff, A. (1995). Pemphigus vulgaris and pregnancy. Obstetrical & gynecological survey 50, 755-760.
Ruiz-Arguelles, A., Brito, G. J., Reyes-Izquierdo, P., Perez-Romano, B., and Sanchez-Sosa, S. (2007). Apoptosis of melanocytes in vitiligo results from antibody penetration. Journal of autoimmunity 29, 281-286.
Ruiz-Arguelles, A., Garcia-Carrasco, M., Jimenez-Brito, G., Sanchez-Sosa, S., Perez-Romano, B., Garces-Eisele, J., Camacho-Alarcon, C., Reyes-Nunez, V., Sandoval-Cruz, M., Mendoza-Pinto, C., et al. (2013). Treatment of vitiligo with a chimeric monoclonal antibody to CD20: a pilot study. Clinical and experimental immunology 174, 229-236.
Saadoun, S., Waters, P., Bell, B. A., Vincent, A., Verkman, A. S., and Papadopoulos, M. C. (2010). Intracerebral injection of neuromyelitis optica immunoglobulin G and human complement produces neuromyelitis optica lesions in mice. Brain: a journal of neurology 133, 349-361.
Sabroe, R. A., Fiebiger, E., Francis, D. M., Maurer, D., Seed, P. T., Grattan, C. E., Black, A. K., Stingl, G., Greaves, M. W., and Barr, R. M. (2002). Classification of anti-FcepsilonRI and anti-IgE autoantibodies in chronic idiopathic urticaria and correlation with disease severity. The Journal of allergy and clinical immunology 110, 492-499.
Sacchi, S., Novellis, V., Paolone, G., Nuzzo, T., lannotta, M., Belardo, C., Squillace, M., Bolognesi, P., Rosini, E., Motta, Z., et al. (2017). Olanzapine, but not clozapine, increases glutamate release in the prefrontal cortex of freely moving mice by inhibiting D-aspartate oxidase activity. Scientific reports 7, 46288.
Saini, S. S., MacGlashan, D. W., Jr., Sterbinsky, S. A., Togias, A., Adelman, D. C., Lichtenstein, L. M., and Bochner, B. S. (1999). Down-regulation of human basophil IgE and FC epsilon RI alpha surface densities and mediator release by anti-IgE-infusions is reversible in vitro and in vivo. Journal of immunology (Baltimore, Md.: 1950) 162, 5624-5630.
Salvi, M., Vannucchi, G., and Beck-Peccoz, P. (2013). Potential utility of rituximab for Graves' orbitopathy. The Journal of clinical endocrinology and metabolism 98, 4291-4299.
Samuels, J., Ng, Y. S., Coupillaud, C., Paget, D., and Meffre, E. (2005). Human B cell tolerance and its failure in rheumatoid arthritis. Annals of the New York Academy of Sciences 1062, 116-126.
Schmidt, E., Obe, K., Brocker, E. B., and Zillikens, D. (2000). Serum levels of autoantibodies to BP180 correlate with disease activity in patients with bullous pemphigoid. Arch Dermatol 136, 174-178.
Sebode, M., Weiler-Normann, C., Liwinski, T., and Schramm, C. (2018). Autoantibodies in Autoimmune Liver Disease-Clinical and Diagnostic Relevance. Frontiers in immunology 9, 609.
Serafini, B., Rosicarelli, B., Magliozzi, R., Stigliano, E., and Aloisi, F. (2004). Detection of ectopic B-cell follicles with germinal centers in the meninges of patients with secondary progressive multiple sclerosis. Brain pathology (Zurich, Switzerland) 14, 164-174.
Serreze, D. V., Fleming, S. A., Chapman, H. D., Richard, S. D., Leiter, E. H., and Tisch, R. M. (1998). B lymphocytes are critical antigen-presenting cells for the initiation of T cell-mediated autoimmune diabetes in nonobese diabetic mice. Journal of immunology (Baltimore, Md.: 1950) 161, 3912-3918.
Shen, P., and Fillatreau, S. (2015). Antibody-independent functions of B cells: a focus on cytokines. Nature reviews Immunology 15, 441-451.
Shen, P., Roch, T., Lampropoulou, V., O'Connor, R. A., Stervbo, U., Hilgenberg, E., Ries, S., Dang, V. D., Jaimes, Y., Daridon, C., et al. (2014). IL-35-producing B cells are critical regulators of immunity during autoimmune and infectious diseases. Nature 507, 366-370.
Shi, J., Hou, S., Fang, Q., Liu, X., Liu, X., and Qi, H. (2018). PD-1 Controls Follicular T Helper Cell Positioning and Function. Immunity 49, 264-274.e264.
Shulman, N. R., Marder, V. J., and Weinrach, R. S. (1965). Similarities between known antiplatelet antibodies and the factor responsible for thrombocytopenia in idiopathic purpura. Physiologic, serologic and isotopic studies. Annals of the New York Academy of Sciences 124, 499-542.
Simon, V. M., Parra, A., Minarro, J., Arenas, M. C., Vinader-Caerols, C., and Aguilar, M. A. (2000). Predicting how equipotent doses of chlorpromazine, haloperidol, sulpiride, raclopride and clozapine reduce locomotor activity in mice. European neuropsychopharmacology: the journal of the European College of Neuropsychopharmacology 10, 159-164.
Singh, I., and Hershman, J. M. (2016). Pathogenesis of Hyperthyroidism. Comprehensive Physiology 7, 67-79.
Smith, M. J., Rihanek, M., Coleman, B. M., Gottlieb, P. A., Sarapura, V. D., and Cambier, J. C. (2018). Activation of thyroid antigen-reactive B cells in recent onset autoimmune thyroid disease patients. Journal of autoimmunity 89, 82-89.
Smith, M. J., Simmons, K. M., and Cambier, J. C. (2017). B cells in type 1 diabetes mellitus and diabetic kidney disease. Nature reviews Nephrology 13, 712-720.
Sollid, L. M. (2017). The roles of MHC class II genes and post-translational modification in celiac disease. Immunogenetics 69, 605-616.
Staubach, P., Onnen, K., Vonend, A., Metz, M., Siebenhaar, F., Tschentscher, I., Opper, B., Magerl, M., Ludtke, R., Kromminga, A., et al. (2006). Autologous whole blood injections to patients with chronic urticaria and a positive autologous serum skin test: a placebo-controlled trial. Dermatology (Basel, Switzerland) 212, 150-159.
Steinweg, S. A., and Gaspari, A. A. (2015). Rituximab for the Treatment of Recalcitrant Chronic Autoimmune Urticaria. Journal of drugs in dermatology: JDD 14, 1387.
Stoecker, Z. R., George, W. T., O'Brien, J. B., Jancik, J., Colon, E., and Rasimas, J. J. (2017). Clozapine usage increases the incidence of pneumonia compared with risperidone and the general population: a retrospective comparison of clozapine, risperidone, and the general population in a single hospital over 25 months. International clinical psychopharmacology 32, 155-160.
Stohl, W., Hiepe, F., Latinis, K. M., Thomas, M., Scheinberg, M. A., Clarke, A., Aranow, C., Wellborne, F. R., Abud-Mendoza, C., Hough, D. R., et al. (2012). Belimumab reduces autoantibodies, normalizes low complement levels, and reduces select B cell populations in patients with systemic lupus erythematosus. Arthritis and rheumatism 64, 2328-2337.
Strippel, C., Monig, C., Golombeck, K. S., Dik, A., Bonte, K., Kovac, S., Schulte-Mecklenbeck, A., Wiendl, H., Meuth, S. G., Johnen, A., et al. (2017). Treating refractory post-herpetic anti-N-methyl-d-aspartate receptor encephalitis with rituximab. Oxford medical case reports 2017, omx034.
Stuart, J. M., and Dixon, F. J. (1983). Serum transfer of collagen-induced arthritis in mice. The Journal of experimental medicine 158, 378-392.
Suan, D., Sundling, C., and Brink, R. (2017). Plasma cell and memory B cell differentiation from the germinal center. Current opinion in immunology 45, 97-102.
Sugimura, T., Shiokawa, S., Haraoka, S., Fujimoto, K., Ohshima, K., Nakamuta, M., and Nishimura, J. (2003). Local antigen-driven oligoclonal expansion of B cells in the liver portal areas of patients with primary biliary cirrhosis. Liver international: official journal of the International Association for the Study of the Liver 23, 323-328.
Sun, L., Erxun, K., Li, J., Yang, J., and Han, C. (2014). Correlations between Anti-Mast Cell Autoantibodies and Chronic Idiopathic Urticaria. Annals of dermatology 26, 145-149.
Sun, W., Meednu, N., Rosenberg, A., Rangel-Moreno, J., Wang, V., Glanzman, J., Owen, T., Zhou, X., Zhang, H., Boyce, B. F., et al. (2018). B cells inhibit bone formation in rheumatoid arthritis by suppressing osteoblast differentiation. Nature communications 9, 5127.
Sun, Y., Liu, Z., Liu, Y., and Li, X. (2015). Increased frequencies of memory and activated B cells and follicular helper T cells are positively associated with high levels of activation induced cytidine deaminase in patients with immunoglobulin A nephropathy. Molecular medicine reports 12, 5531-5537.
Suurmond, J., Atisha-Fregoso, Y., Marasco, E., Barley, A. N., Ahmed, N., Calderon, S. A., Wong, M. Y., Mackay, M. C., Aranow, C., and Diamond, B. (2018). Loss of an IgG plasma cell checkpoint in patients with lupus. The Journal of allergy and clinical immunology.
Suzuki, H., Fan, R., Zhang, Z., Brown, R., Hall, S., Julian, B. A., Chatham, W. W., Suzuki, Y., Wyatt, R. J., Moldoveanu, Z., et al. (2009). Aberrantly glycosylated IgA1 in IgA nephropathy patients is recognized by IgG antibodies with restricted heterogeneity. The Journal of clinical investigation 119, 1668-1677.
Suzuki, Y., and Tomino, Y. (2008). Potential immunopathogenic role of the mucosa-bone marrow axis in IgA nephropathy: insights from animal models. Seminars in nephrology 28, 66-77.
Sveinsson, O., Granqvist, M., Forslin, Y., Blennow, K., Zetterberg, H., and Piehl, F. (2017). Successful combined targeting of B- and plasma cells in treatment refractory anti-NMDAR encephalitis. Journal of neuroimmunology 312, 15-18.
Svensson, L., Jirholt, J., Holmdahl, R., and Jansson, L. (1998). B cell-deficient mice do not develop type II collagen-induced arthritis (CIA). Clinical and experimental immunology 111, 521-526.
Takahashi, H. (2017). Desmoglein 3-Reactive B Cells “Hiding” in Pemphigus Lesions. The Journal of investigative dermatology 137, 2255-2257.
Takahashi, T., Miura, T., Nakamura, J., Yamada, S., Miura, T., Yanagi, M., Matsuda, Y., Usuda, H., Emura, I., Tsuneyama, K., et al. (2012). Plasma cells and the chronic nonsuppurative destructive cholangitis of primary biliary cirrhosis. Hepatology (Baltimore, Md.) 55, 846-855.
Tanaka, N., and Glass, V. B. (1970). Effect of prolonged administration of parietal cell antibodies from patients with atrophic gastritis and pernicious anemia on the parietal cell mass and hydrochloric acid output in rats. Gastroenterology 58, 482-494.
Tanyeri, M. H., Buyukokuroglu, M. E., Tanyeri, P., Mutlu, O., Akar, F. Y., Ulak, G., and Erden, B. F. (2017). Effects of long-term treatment with haloperidol, clozapine and aripiprazole on mice isolated vas deferens. International urology and nephrology 49, 1561-1567.
Tellier, J., and Nutt, S. L. (2018). Plasma cells: the programming of an antibody-secreting machine. European journal of immunology.
Tobin, D. J. (2003). Characterization of hair follicle antigens targeted by the anti-hair follicle immune response. The journal of investigative dermatology Symposium proceedings 8, 176-181.
Tobin, D. J., Alhaidari, Z., and Olivry, T. (1998). Equine alopecia areata autoantibodies target multiple hair follicle antigens and may alter hair growth. A preliminary study. Experimental dermatology 7, 289-297.
Tobin, D. J., Gardner, S. H., Lindsey, N. J., Hoffmann, R., Happle, R., and Freyschmidt-Paul, P. (2002). Diphencyprone immunotherapy alters anti-hair follicle antibody status in patients with alopecia areata. European journal of dermatology: EJD 12, 327-334.
Tobin, D. J., Gardner, S. H., Luther, P. B., Dunston, S. M., Lindsey, N. J., and Olivry, T. (2003). A natural canine homologue of alopecia areata in humans. The British journal of dermatology 149, 938-950.
Tobin, D. J., Orentreich, N., Bystryn, J.-C., and Perelman, T. R. O. (1994a). Autoantibodies to Hair Follicles in Normal Individuals. Archives of Dermatology 130, 395-396.
Tobin, D. J., Orentreich, N., Fenton, D. A., and Bystryn, J. C. (1994b). Antibodies to hair follicles in alopecia areata. The Journal of investigative dermatology 102, 721-724.
Tobin, D. J., Sundberg, J. P., King, L. E., Jr., Boggess, D., and Bystryn, J. C. (1997). Autoantibodies to hair follicles in C3H/HeJ mice with alopecia areata-like hair loss. The Journal of investigative dermatology 109, 329-333.
Tomana, M., Novak, J., Julian, B. A., Matousovic, K., Konecny, K., and Mestecky, J. (1999). Circulating immune complexes in IgA nephropathy consist of IgA1 with galactose-deficient hinge region and antiglycan antibodies. The Journal of clinical investigation 104, 73-81.
Tong, L. J., Balakrishnan, G., Kochan, J. P., Kinet, J. P., and Kaplan, A. P. (1997). Assessment of autoimmunity in patients with chronic urticaria. The Journal of allergy and clinical immunology 99, 461-465.
Toosi, S., Collins, J. W., Lohse, C. M., Wolz, M. M., Wieland, C. N., Camilleri, M. J., Bruce, A. J., McEvoy, M. T., and Lehman, J. S. (2016). Clinicopathologic features of IgG/IgA pemphigus in comparison with classic (IgG) and IgA pemphigus. International journal of dermatology 55, e184-190.
Toubi, E., Adir-Shani, A., Kessel, A., Shmuel, Z., Sabo, E., and Hacham, H. (2000). Immune aberrations in B and T lymphocytes derived from chronic urticaria patients. Journal of clinical immunology 20, 371-378.
Toyka, K. V., Brachman, D. B., Pestronk, A., and Kao, I. (1975). Myasthenia gravis: passive transfer from man to mouse. Science (New York, N.Y.) 190, 397-399.
Tozzoli, R., Kodermaz, G., Perosa, A. R., Tampoia, M., Zucano, A., Antico, A., and Bizzaro, N. (2010). Autoantibodies to parietal cells as predictors of atrophic body gastritis: a five-year prospective study in patients with autoimmune thyroid diseases. Autoimmunity reviews 10, 80-83.
Trueb, R. M., and Dias, M. (2018). Alopecia Areata: a Comprehensive Review of Pathogenesis and Management. Clinical reviews in allergy & immunology 54, 68-87.
Tsubakio, T., Kurata, Y., Kanayama, Y., Yonezawa, T., Tarui, S., and Kitani, T. (1983). In vitro platelet phagocytosis in idiopathic thrombocytopenic purpura. Acta haematologica 70, 250-256.
Tsuda, M., Moritoki, Y., Lian, Z.-X., Zhang, W., Yoshida, K., Wakabayashi, K., Yang, G.-X., Nakatani, T., Vierling, J., Lindor, K., et al. (2012). Biochemical and immunologic effects of rituximab in patients with primary biliary cirrhosis and an incomplete response to ursodeoxycholic acid. Hepatology (Baltimore, Md.) 55, 512-521.
Tsuneyama, K., Baba, H., Morimoto, Y., Tsunematsu, T., and Ogawa, H. (2017). Primary Biliary Cholangitis: Its Pathological Characteristics and Immunopathological Mechanisms. The journal of medical investigation: JMI 64, 7-13.
Ueki, I., Abiru, N., Kobayashi, M., Nakahara, M., Ichikawa, T., Eguchi, K., and Nagayama, Y. (2011). B cell-targeted therapy with anti-CD20 monoclonal antibody in a mouse model of Graves' hyperthyroidism. Clinical and experimental immunology 163, 309-317.
Utsunomiya, N., Chino, T., Oyama, N., Utsunomiya, A., Yamaguchi, Y., Takashima, W., Tokuriki, A., and Hasegawa, M. (2017). Sublamina densa-type linear IgA bullous dermatosis with IgA autoantibodies specific for type VII collagen: a case report and clinicopathological review of 32 cases. Dermatology online journal 23.
van de Wal, Y., Kooy, Y. M., van Veelen, P. A., Pena, S. A., Mearin, L. M., Molberg, O., Lundin, K. E., Sollid, L. M., Mutis, T., Benckhuijsen, W. E., et al. (1998). Small intestinal T cells of celiac disease patients recognize a natural pepsin fragment of gliadin. Proceedings of the National Academy of Sciences of the United States of America 95, 10050-10054.
van den Wall Bake, A. W., Daha, M. R., Radl, J., Haaijman, J. J., Van der Ark, A., Valentijn, R. M., and Van Es, L. A. (1988). The bone marrow as production site of the IgA deposited in the kidneys of patients with IgA nephropathy. Clinical and experimental immunology 72, 321-325.
Van der Weerd, K., Van Hagen, P. M., Schrijver, B., Kwekkeboom, D. J., De Herder, W. W., Ten Broek, M. R., Postema, P. T., Van Dongen, J. J., Staal, F. J., and Dik, W. A. (2013). The peripheral blood compartment in patients with Graves' disease: activated T lymphocytes and increased transitional and pre-naive mature B lymphocytes. Clinical and experimental immunology 174, 256-264.
Vander Heiden, J. A., Stathopoulos, P., Zhou, J. Q., Chen, L., Gilbert, T. J., Bolen, C. R., Barohn, R. J., Dimachkie, M. M., Ciafaloni, E., Broering, T. J., et al. (2017). Dysregulation of B Cell Repertoire Formation in Myasthenia Gravis Patients Revealed through Deep Sequencing. Journal of immunology (Baltimore, Md.: 1950) 198, 1460-1473.
Vannucchi, G., Covelli, D., Curro, N., Dazzi, D., Maffini, A., Campi, I., Bonara, P., Guastella, C., Pignataro, L., Ratiglia, R., et al. (2012). Serum BAFF concentrations in patients with Graves' disease and orbitopathy before and after immunosuppressive therapy. The Journal of clinical endocrinology and metabolism 97, E755-759.
Vergani, D., Mieli-Vergani, G., Mondelli, M., Portmann, B., and Eddleston, A. L. (1987). Immunoglobulin on the surface of isolated hepatocytes is associated with antibody-dependent cell-mediated cytotoxicity and liver damage. Liver 7, 307-315.
Viisanen, T., Ihantola, E. L., Nanto-Salonen, K., Hyoty, H., Nurminen, N., Selvenius, J., Juutilainen, A., Moilanen, L., Pihlajamaki, J., Veijola, R., et al. (2017). Circulating CXCR5+PD-1+ICOS+ Follicular T Helper Cells Are Increased Close to the Diagnosis of Type 1 Diabetes in Children With Multiple Autoantibodies. Diabetes 66, 437-447.
Vincent, A., Newsom-Davis, J., Newton, P., and Beck, N. (1983). Acetylcholine receptor antibody and clinical response to thymectomy in myasthenia gravis. Neurology 33, 1276-1282.
Vincent, A., Scadding, G. K., Thomas, H. C., and Newsom-Davis, J. (1978). In-vitro synthesis of anti-acetylcholine-receptor antibody by thymic lymphocytes in myasthenia gravis. Lancet (London, England) 1, 305-307.
Wallace, Z. S., Mattoo, H., Carruthers, M., Mahajan, V. S., Della Torre, E., Lee, H., Kulikova, M., Deshpande, V., Pillai, S., and Stone, J. H. (2015). Plasmablasts as a biomarker for IgG4-related disease, independent of serum IgG4 concentrations. Annals of the rheumatic diseases 74, 190-195.
Wang, L., Sun, X., Qiu, J., Cai, Y., Ma, L., Zhao, P., and Jiang, Y. (2015). Increased numbers of circulating ICOS(+) follicular helper T and CD38(+) plasma cells in patients with newly diagnosed primary biliary cirrhosis. Digestive diseases and sciences 60, 405-413.
Wang, Y. Y., Zhang, L., Zhao, P. W., Ma, L., Li, C., Zou, H. B., and Jiang, Y. F. (2014). Functional implications of regulatory B cells in human IgA nephropathy. Scandinavian journal of immunology 79, 51-60.
Watanabe, R., Ishiura, N., Nakashima, H., Yazawa, N., Kuwano, Y., Tada, Y., Okochi, H., Fujimoto, M., and Tamaki, K. (2007). Increased serum levels of circulating CD40 ligand in patients with bullous pemphigoid: preliminary results. Dermatology (Basel, Switzerland) 215, 180-186.
Wehr, C., Kivioja, T., Schmitt, C., Ferry, B., Witte, T., Eren, E., Vlkova, M., Hernandez, M., Detkova, D., Bos, P. R., et al. (2008). The EUROclass trial: defining subgroups in common variable immunodeficiency. Blood 111, 77-85.
Williams, A. A., Ingram, W. M., Levine, S., Resnik, J., Kamel, C. M., Lish, J. R., Elizalde, D. I., Janowski, S. A., Shoker, J., Kozlenkov, A., et al. (2012). Reduced levels of serotonin 2A receptors underlie resistance of Egr3-deficient mice to locomotor suppression by clozapine. Neuropsychopharmacology: official publication of the American College of Neuropsychopharmacology 37, 2285-2298.
Williams, R. O. (2012). What Have We Learned about the Pathogenesis of Rheumatoid Arthritis from TNF-Targeted Therapy? ISRN Immunology 2012, 15.
Winqvist, O., Karlsson, F. A., and Kampe, 0. (1992). 21-Hydroxylase, a major autoantigen in idiopathic Addison's disease. Lancet (London, England) 339, 1559-1562.
Wolfe, G. I., Kaminski, H. J., Aban, I. B., Minisman, G., Kuo, H. C., Marx, A., Strobel, P., Mazia, C., Oger, J., Cea, J. G., et al. (2016). Randomized Trial of Thymectomy in Myasthenia Gravis. The New England journal of medicine 375, 511-522.
Yanaba, K., Hamaguchi, Y., Venturi, G. M., Steeber, D. A., St Clair, E. W., and Tedder, T. F. (2007). B cell depletion delays collagen-induced arthritis in mice: arthritis induction requires synergy between humoral and cell-mediated immunity. Journal of immunology (Baltimore, Md.: 1950) 179, 1369-1380.
Yu, X. Q., Li, M., Zhang, H., Low, H. Q., Wei, X., Wang, J. Q., Sun, L. D., Sim, K. S., Li, Y., Foo, J. N., et al. (2011). A genome-wide association study in Han Chinese identifies multiple susceptibility loci for IgA nephropathy. Nature genetics 44, 178-182.
Yurasov, S., Wardemann, H., Hammersen, J., Tsuiji, M., Meffre, E., Pascual, V., and Nussenzweig, M. C. (2005). Defective B cell tolerance checkpoints in systemic lupus erythematosus. The Journal of experimental medicine 201, 703-711.
Zanoni, G., Navone, R., Lunardi, C., Tridente, G., Bason, C., Sivori, S., Beri, R., Dolcino, M., Valletta, E., Corrocher, R., et al. (2006). In celiac disease, a subset of autoantibodies against transglutaminase binds toll-like receptor 4 and induces activation of monocytes. PLoS medicine 3, e358.
Zehentmeier, S., Roth, K., Cseresnyes, Z., Sercan, O., Horn, K., Niesner, R. A., Chang, H. D., Radbruch, A., and Hauser, A. E. (2014). Static and dynamic components synergize to form a stable survival niche for bone marrow plasma cells. European journal of immunology 44, 2306-2317.
Zha, B., Huang, X., Lin, J., Liu, J., Hou, Y., and Wu, G. (2014). Distribution of lymphocyte subpopulations in thyroid glands of human autoimmune thyroid disease. Journal of clinical laboratory analysis 28, 249-254.
Zhang, J., Zhang, W., Leung, P. S., Bowlus, C. L., Dhaliwal, S., Coppel, R. L., Ansari, A. A., Yang, G. X., Wang, J., Kenny, T. P., et al. (2014). Ongoing activation of autoantigen-specific B cells in primary biliary cirrhosis. Hepatology (Baltimore, Md.) 60, 1708-1716.
Zhao, N., Hou, P., Lv, J., Moldoveanu, Z., Li, Y., Kiryluk, K., Gharavi, A. G., Novak, J., and Zhang, H. (2012). The level of galactose-deficient IgA1 in the sera of patients with IgA nephropathy is associated with disease progression. Kidney international 82, 790-796.
Ziegler, A. G., Rewers, M., Simell, O., Simell, T., Lempainen, J., Steck, A., Winkler, C., Ilonen, J., Veijola, R., Knip, M., et al. (2013). Seroconversion to multiple islet autoantibodies and risk of progression to diabetes in children. Jama 309, 2473-2479.
Zone, J. J., Egan, C. A., Taylor, T. B., and Meyer, L. J. (2004). IgA autoimmune disorders: development of a passive transfer mouse model. The journal of investigative dermatology Symposium proceedings 9, 47-51.

1. A method of treating or preventing a pathogenic immunoglobulin driven B cell disease in a subject comprising administering to the subject an effective amount of a compound selected from clozapine, norclozapine and prodrugs thereof and pharmaceutically acceptable salts and solvates thereof, wherein said compound causes mature B cells to be inhibited in said subject.

2. The method according to claim 1 wherein the compound is clozapine or a pharmaceutically acceptable salt or solvates thereof.

3. The method according to claim 1 wherein the mature B cells are class switched memory B cells.

4. The method according to claim 1 wherein the mature B cells are plasmablasts.

5. The method according to claim 1 wherein the pathogenic immunoglobulin driven B cell disease is a pathogenic IgG driven B cell disease.

6. The method according to claim 1 wherein the pathogenic immunoglobulin driven B cell disease is a disease selected from the group consisting of Pemphigus vulgaris, Pemphigus foliaceus, bullous pemphigoid, cicatricial pemphigoid, autoimmune alopecia, vitiligo, dermatitis herpetiformis, chronic autoimmune urticaria, coeliac disease, Graves' disease, Hashimoto's thyroiditis, Type 1 diabetes mellitus, autoimmune Addison's disease, autoimmune haemolytic anaemia, autoimmune thrombocytopenic purpura, cryoglobulinemia, pernicious anaemia, myasthenia gravis, multiple sclerosis, neuromyelitis optica, autoimmune epilepsy and encephalitis, autoimmune hepatitis, primary biliary cirrhosis and primary sclerosing cholangitis.

7. The method according to claim 6 wherein the pathogenic immunoglobulin driven B cell disease is a disease selected from the group consisting of Pemphigus vulgaris, Pemphigus foliaceus and bullous pemphigoid.

8. The method according to claim 1 wherein the pathogenic immunoglobulin driven B cell disease is a pathogenic IgA driven B cell disease.

9. The method according to claim 1 wherein the pathogenic immunoglobulin driven B cell disease is a disease selected from the group consisting of dermatitis herpetiformis, linear IgA disease, coeliac disease, IgA nephropathy, Pemphigus vulgaris, Pemphigus foliaceus, cicatricial pemphigoid and bullous pemphigoid.

10. The method according to claim 9 wherein the pathogenic immunoglobulin driven B cell disease is a disease selected from the group consisting of dermatitis herpetiformis and linear IgA disease.

11. A method of treating or preventing a pathogenic immunoglobulin driven B cell disease in a subject comprising administering to the subject an effective amount of a pharmaceutical composition comprising a compound selected from clozapine, norclozapine and prodrugs thereof and pharmaceutically acceptable salts and solvates thereof; and a pharmaceutically acceptable diluent or carrier, wherein said compound causes mature B cells to be inhibited in said subject.

12. The method according to claim 11 wherein the pharmaceutical composition is administered orally.

13. The method according to claim 11 wherein the mature B cells are class switched memory B cells or plasmablasts.

14. The method according to claim 1 wherein the compound is administered in combination with a second or further therapeutic agent for the treatment or prevention of a pathogenic immunoglobulin driven B cell disease.

15. The method according to claim 14 wherein the second or further substance for the treatment or prevention of a pathogenic immunoglobulin driven B cell disease is selected from anti-TNFα agents (such as anti-TNFα antibodies e.g. infliximab or adalumumab), calcineurin inhibitors (such as tacrolimus or cyclosporine), antiproliferative agents (such as mycophenolate e.g. as mofetil or sodium, or azathioprine), general anti-inflammatories (such as hydroxychloroquine or NSAIDS such as ketoprofen and colchicine), mTOR inhibitors (such as sirolimus), steroids (such as prednisone), anti-CD80/CD86 agents (such as abatacept), anti-CD-20 agents (such as anti-CD-20 antibodies e.g. rituximab). anti-BAFF agents (such as anti-BAFF antibodies e.g. tabalumab or belimumab, or atacicept), immunosuppressants (such as methotrexate or cyclophosphamide), anti-FcRn agents (e.g. anti-FcRn antibodies) and other antibodies (such as ARGX-113, PRN-1008, SYNT-001, veltuzumab, ocrelizumab, ofatumumab, obinutuzumab, ublituximab, alemtuzumab, milatuzumab, epratuzumab and blinatumomab).

16-23. (canceled)
